(19) United States
(12) Patent Application Publication
(10) Pub. No.: US 2025/0368624 A1
(43) Pub. Date: Dec. 4, 2025

(54) M24B AMINOPEPTIDASE INHIBITORS FOR CARD8 INFLAMMASOME ACTIVATION

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Daniel Bachovchin, New York, NY (US); Qinghui Wang, New York, NY (US); Qifeng Chen, New York, NY (US)

(73) Assignees: Memorial Sloan-Kettering Cancer Center, New York, NY (US); Memorial Hospital for Cancer and Allied Diseases, New York, NY (US); Sloan-Kettering Institute for Cancer Research, New York, NY (US)

(21) Appl. No.: 19/117,893

(22) PCT Filed: Oct. 4, 2023
(86) PCT No.: PCT/US2023/075989

§ 371 Date: Apr. 2, 2025

Related U.S. Application Data
(60) Provisional application No. 63/378,507, filed on Oct. 5, 2022.

(51) Int. Cl. C07D 403/06; C07C 237/12; C07D 207/06; C07D 207/16; C07D 403/04; C07D 403/12

(52) U.S. Cl. CPC C07D 403/06; C07C 237/12; C07D 207/06; C07D 207/16; C07D 403/04; C07D 403/12

(57) ABSTRACT

The present technology relates generally to compounds for inhibiting M24B aminopeptidases to selectively activating the CARDS inflammasome, an innate immune signaling platform that triggers pyroptotic cell death.

### CROSS-REFERENCE TO RELATED APPLICATIONS

This application claims the benefit of and priority to U.S. Provisional Application No. 63/378,507, filed Oct. 5, 2022, the contents of which are incorporated herein by reference in their entirety for any and all purposes.

### U.S. GOVERNMENT SUPPORT

This invention was made with government support under R01CA266478 awarded by the National Institutes of Health. The government has certain rights in the invention.

### TECHNICAL FIELD

The present technology relates generally to compounds that inhibit M24B aminopeptidases.

### SUMMARY

In an aspect, the present disclosure provides a compound according to Formula I

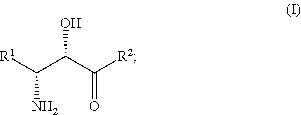

or a pharmaceutically acceptable salt and/or solvate thereof, wherein
R¹ is C₁-C₆ alkyl, —(CH₂)ₙ—(R⁴), cycloalkyl, adamantly, heterocyclyl, aryl, aralkyl, or heteroaryl;
n is 1, 2, 3, 4, 5, or 6;
R² is heterocyclyl, alkylamino carboxylate alkyl ester, or aralkylamino carboxylate alkyl ester, optionally where R² is heterocyclyl substituted with one or more of carboxylate alkyl ester, hydroxide, halogen, cyclo, alkyl, amide, alkylamido, alkylcarbamoyl, alkylsulfonamido, tetrazole, carbonyl amino acid, or carboxy alkylamido; and
R⁴ is trifluoromethyl or hydroxide.

Further aspects and embodiments of the present technology are described herein.

### BRIEF DESCRIPTION OF THE DRAWING

FIG. 1 provides cytotoxicity data of N-terminal analogs of compounds of the present invention. WT and CASP1^{-/-} MV4;11 cells were treated with the indicated compounds (100 μM to 15.2 nM, 3-fold dilutions) for 24 h before CellTiter-Glo (CTG) analysis.

FIG. 2 provides cytotoxicity of C-terminal analogs of compounds of the present invention. WT and CASP1^{-/-} MV4;11 cells were treated with the indicated compounds (100 μM to 15.2 nM, 3-fold dilutions) for 24 h before CTG analysis.

FIGS. 3A-3G demonstrate inhibition of PEPD and XPNPEP1 inhibitors with pseudo-tripeptides analogs of compounds of the present invention. FIG. 3A provides the percent inhibition of PEPD and XPNPEP1 enzymatic activity or cell line viability (as determined by CTG) after treatment with the indicated compounds (1 μM for PEPD, MV4;11, and OC-AML2 assays; 20 μM for XPNPEP1 assay). FIGS. 3B and 3C provide inhibition of recombinant PEPD (FIG. 3B) or XPNPEP1 (FIG. 3C) activity by the indicated compounds. FIG. 3D-3F provide the viability of MV4;11 WT (FIGS. 3D, 3E) or CASP1^{-/-} (FIG. 3F) cells after treatment with the indicated compounds (24 h) as assessed by CTG (FIGS. 3D, 3F) or Cytotox-Flour (CTF) assays (FIG. 3E). FIG. 3G OCI-AML2 cells were treated with indicated compounds (6.25 μM) incubated for 6 h before staining with PI. PI uptake was recorded for 6 h. Data in FIG. 3A (n=3) are means of biological replicates; data in FIGS. 3B-3G (n=3) are means±SEM of biological replicates.

FIGS. 4A-4I demonstrate CQ80 selectively activates the CARD8 inflammasome. FIG. 4A shows MV4;11 cells incubated with compounds (6.25 μM) for 6 h before monitoring for PI uptake. FIG. 4B shows OCI-AML2 cells treated with CQ31 or CQ80 for 24 h before assessing cell viability by CTG. FIGS. 3C-3F show cells treated with CQ31 or CQ80 (20 μM) for 24 h before LDH release and immunoblot analyses. FIG. 3G show N/TERT-1 immortalized keratinocytes treated with VbP (10 μM) or the indicated CQ compounds (20 μM) for 24 h before CTG analyses. FIGS. 3H and 3I show BMDMs from C57BL/6J mice (FIG. 3H) or RAW264.7 cells (FIG. 3I) treated with VbP (10 μM), CQ31 (20 μM), or CQ80 (20 μM) for 24 h before LDH release and/or immunoblot analyses._Data in FIGS. 3A-3E, 3G, and 3I (n=3) are means±SEM of biological replicates. ***p<0.001, **p<0.01, *p<0.05 by two-sided Students t-test. N.s., not significant.

FIG. 5 provides MV4;11 cells treated with indicated compounds (10 μM to 1.5 nM, 3-fold dilutions) for 24 h before CTG analysis.

### DETAILED DESCRIPTION

It is to be appreciated that certain aspects, modes, embodiments, variations and features of the present methods are described below in various levels of detail in order to provide a substantial understanding of the present technology.

### Definitions

Unless defined otherwise, all technical and scientific terms used herein generally have the same meaning as commonly understood by one of ordinary skill in the art to which this technology belongs.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, “prevention,” “prevent,” or “preventing” of a disease or condition refers to one or more compounds that, in a statistical sample, reduces the occurrence of the disease or condition in the treated sample relative to an untreated control sample, or delays the onset of one or more symptoms of the disease or condition relative to the untreated control sample. As used herein, prevention includes preventing or delaying the initiation of symptoms of the disease or condition. As used herein, prevention also includes preventing a recurrence of one or more signs or symptoms of a disease or condition.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

Generally, reference to a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P, and ³⁵S are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

As used herein, Cₓ-C_{y}, such as C₁-C₁₂, C₁-C₈, or C₁-C₆ when used before a group refers to that group containing x to y carbon atoms

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups.” Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)R⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O—. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

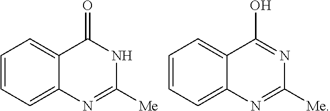

As another example, guanidines may exhibit the following isomeric forms in protic organic solution, also referred to as tautomers of each other:

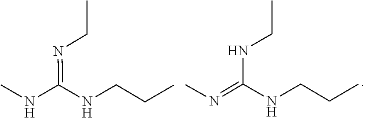

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

Throughout this disclosure, various publications, patents, and published patent specifications are referenced by an identifying citation. Also within this disclosure are Arabic numerals referring to referenced citations, the full bibliographic details of which are provided immediately preceding the claims. The disclosures of these publications, patents, and published patent specifications are hereby incorporated by reference into the present disclosure to more fully describe the state of the art to which this invention pertains.

### The Present Technology

NLRP1 and CARD8 are related intracellular pattern-recognition receptors (PRRs) that detect the same danger-associated signals and assemble into innate immune signaling platforms called inflammasomes. Both inflammasomes may recruit and activate the cysteine protease caspase-1 (CASP1), which in turn cleaves and activates the pore-forming protein GSDMD to meditate a lytic form of cell death called pyroptosis. The NLRP1 inflammasome, but not the CARD8 inflammasome, may also activate CASP1 to cleave and activate the cytokines IL-1β and -18 and thereby induces a more inflammatory form of pyroptosis. As a result, the activation of the NLRP1 inflammasome may be more tightly controlled.

Although they have distinct activation thresholds and inflammatory outputs, NLRP1 and CARD8 have similar domain structures and activation mechanisms. Both proteins undergo autoproteolysis in their function-to-find domains (FIINDs) to create N-terminal (NT) and C-terminal (CT) polypeptide chains that remain non-covalently associated. In this structure, the autoinhibitory NT fragments may prevent the inflammatory CT fragments from self-oligomerizing to nucleate inflammasome formation. The proteasome-mediated degradation of the NT fragment may release the CT fragment from autoinhibition, but freed CT fragments may be next sequestered in a ternary complex with one copy of the full-length PRR and one copy of either DPP8 or 9 (DPP8/9). Potent DPP8/9 inhibitors, including Val-boroPro (VbP), activate the CARD8 and NLRP1 inflammasomes by both accelerating NT degradation and destabilizing the ternary complexes. DPP8/9 are serine proteases that cleave Xaa-Pro dipeptides (where Xaa is any amino acid) from the N-termini of proteasome-generated polypeptides, but how and why DPP8/9 repress inflammasome formation is not yet fully understood.

The compounds of the present technology inhibit the M24B Mn²⁺-dependent metallopeptidases PEPD and XPNPEP1 and selectively activate the CARD8 inflammasome without simultaneously activating the NLRP1 inflammasome. PEPD and XPNPEP1 cleave the N-terminal amino acid preceding proline in dipeptides and longer polypeptides, respectively. As such, the compounds of the present technology may cause the accumulation of various Xaa-Pro-containing peptides (Xaa is any amino acid), which in turn weakly inhibit DPP8/9 in such a way that sufficiently derepresses CARD8, but not NLRP1. Selective CARD8 inflammasome activators are not only useful research tools, but also have potential therapeutic value. For example, without being bound by any theory, such agents may kill CARD8-expressing cancer cells without also triggering the highly inflammatory NLRP1 response in normal tissues.

Thus, in an aspect, the present disclosure provides a compound according to Formula I

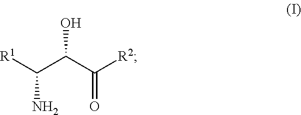

or a pharmaceutically acceptable salt and/or solvate thereof, wherein
R¹ is C₁-C₆ alkyl, —(CH₂)ₙ—(R⁴), cycloalkyl, adamantly, heterocyclyl, aryl, aralkyl, or heteroaryl;
n is 1, 2, 3, 4, 5, or 6;
R² is heterocyclyl, alkylamino carboxylate alkyl ester, or aralkylamino carboxylate alkyl ester, optionally where R² is heterocyclyl substituted with one or more of carboxylate alkyl ester, hydroxide, halogen, cyclo, alkyl, amide, alkylamido, alkylcarbamoyl, alkylsulfonamido, tetrazole, carbonyl amino acid, or carboxy alkylamido; and
R⁴ is trifluoromethyl or hydroxide.

In any embodiment herein, it may be that R¹ is a branched C₁-C₆ alkyl and R₂ is heterocyclyl, alkylamino carboxylate alkyl ester, or aralkylamino carboxylate alkyl ester, optionally where R² is heterocyclyl substituted with one or more of carboxylate alkyl ester, hydroxide, halogen (e.g., fluorine), cyclic, alkyl, amide, alkylamido, alkylcarbamoyl, alkylsulfonamido, tetrazole, carbonyl amino acid, or carboxy alkylamido. For example, R¹ may be a branched C₁-C₆ alkyl. For example, R¹ may be

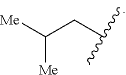 [SMILES: *CC(C)C]

In any embodiment herein, it may be that R¹ is

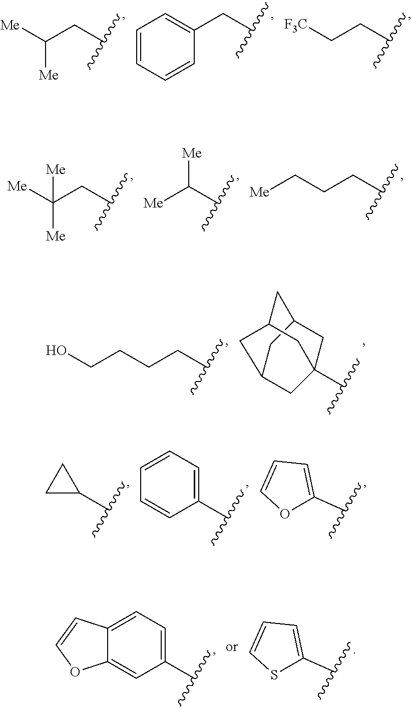

In any embodiment herein, it may be that R² is

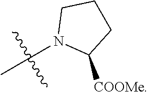 [SMILES: *N1CCC[C@H]1C(=O)OC]

In any embodiment herein, it may be that R² is

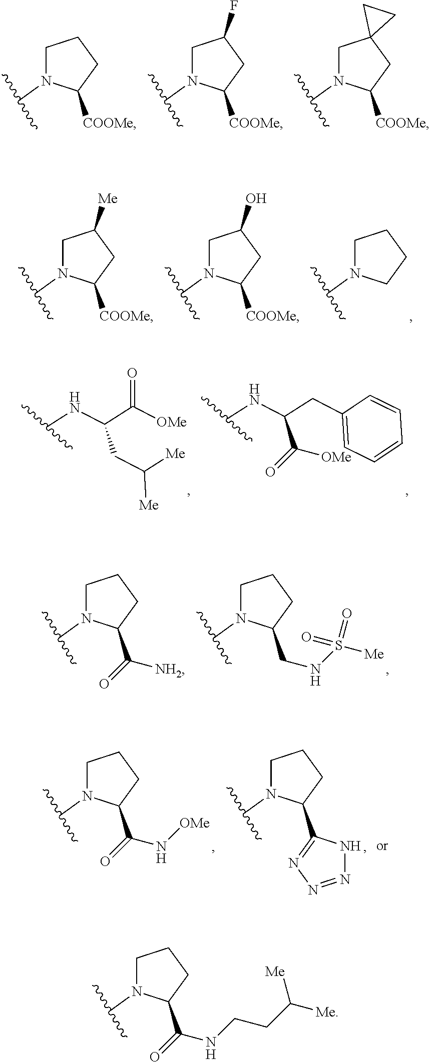

In any embodiment herein, R² may be

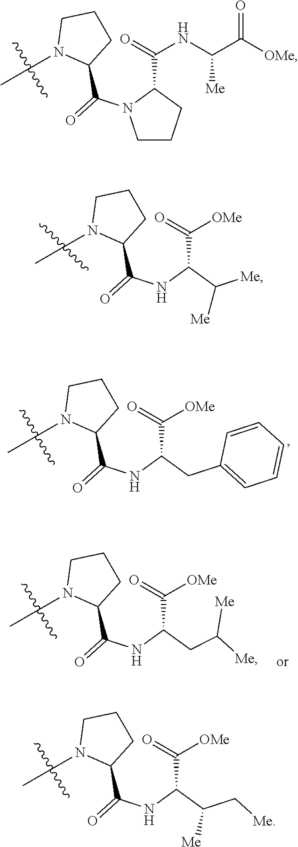 [SMILES: *N1CCC[C@H]1C(=O)N1CCC[C@H]1C(=O)N[C@@H](C)C(=O)OC ; *N1CCC[C@H]1C(=O)N[C@H](C(=O)OC)C(C)C ; *N1CCC[C@H]1C(=O)N[C@@H](Cc1ccccc1)C(=O)OC ; *N1CCC[C@H]1C(=O)N[C@@H](CC(C)C)C(=O)OC ; *N1CCC[C@H]1C(=O)N[C@H](C(=O)OC)[C@@H](C)CC]

In any embodiment herein, it may be that R² is

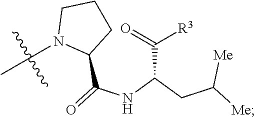

wherein R³ is NH₂, alkylamino, N(Me)₂, N(H)OMe, heterocyclyl, sulfonamido, fluoroalkyl amino, aralkyl amino, or heteroarylalkyl amino. For example, R³ may be aralkyl amino or heteroarylalkyl amino. For example, R³ may be NH₂, N(Me)₂,

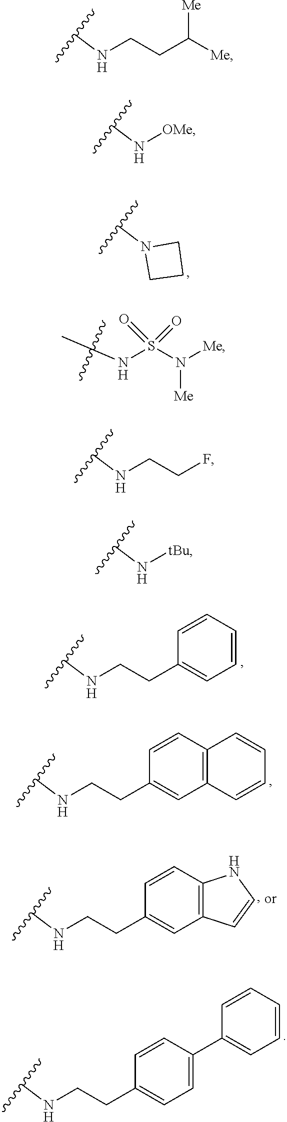

In any embodiment herein, it may be that the compound is of Formula IA

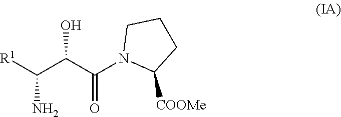

or a pharmaceutically acceptable salt and/or solvate thereof.

In any embodiment herein, it may be that the compound is of Formula IB

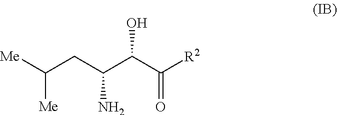

or a pharmaceutically acceptable salt and/or solvate thereof.

In any embodiment herein, it may be that the compound is of Formula IC

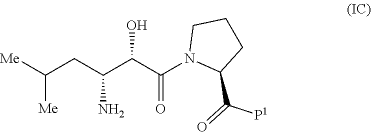

or a pharmaceutically acceptable salt and/or solvate thereof, wherein P¹ is L-Pro-Ala-OMe, L-Val-OMe, L-Phe-OMe, L-Leu-OMe, or L-Ile-OMe.

In any embodiment herein, it may be that the compound is of Formula ID

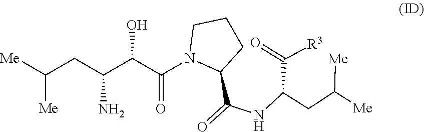

or a pharmaceutically acceptable salt and/or solvate thereof, wherein R³ is NH₂, N(Me)₂,

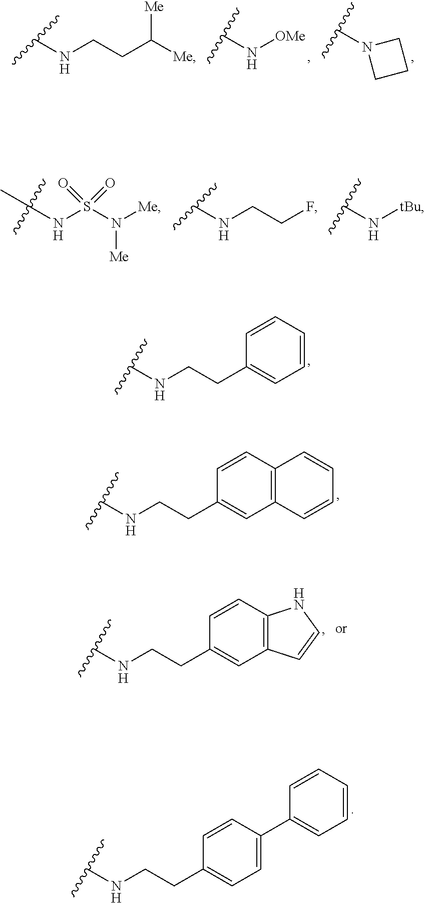

In any embodiment herein, it may be that the compound is any of those listed Table 1, or a pharmaceutically acceptable salt and/or solvate thereof.

TABLE 1
N-Terminal Analogs

| | |
|---|---|
| 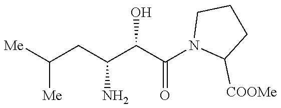 [SMILES: COC(=O)C1CCCN1C(=O)[C@@H](O)[C@H](N)CC(C)C] | 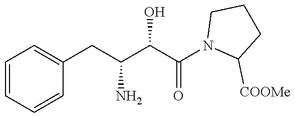 [SMILES: COC(=O)C1CCCN1C(=O)[C@@H](O)[C@H](N)Cc1ccccc1] |
| 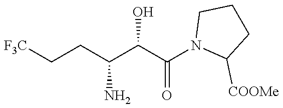 [SMILES: COC(=O)C1CCCN1C(=O)[C@@H](O)[C@H](N)CCC(F)(F)F] | 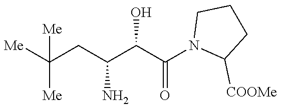 [SMILES: COC(=O)C1CCCN1C(=O)[C@@H](O)[C@H](N)CC(C)(C)C] |
| 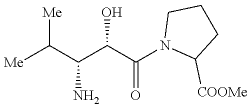 [SMILES: COC(=O)C1CCCN1C(=O)[C@@H](O)[C@H](N)C(C)C] | 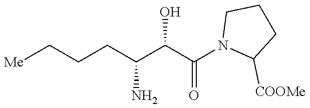 [SMILES: CCCC[C@@H](N)[C@H](O)C(=O)N1CCCC1C(=O)OC] |
| 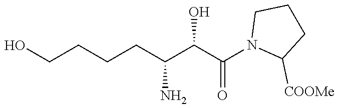 [SMILES: COC(=O)C1CCCN1C(=O)[C@@H](O)[C@H](N)CCCCO] | 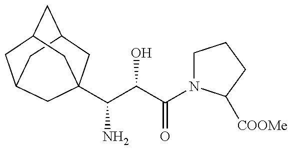 [SMILES: COC(=O)C1CCCN1C(=O)[C@@H](O)[C@H](N)C12CC3CC(CC(C3)C1)C2] |
| 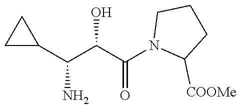 [SMILES: COC(=O)C1CCCN1C(=O)[C@@H](O)[C@H](N)C1CC1] | 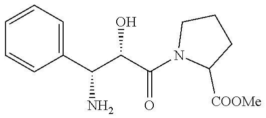 [SMILES: COC(=O)C1CCCN1C(=O)[C@@H](O)[C@H](N)c1ccccc1] |
| 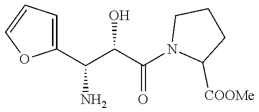 [SMILES: COC(=O)C1CCCN1C(=O)[C@@H](O)[C@H](N)c1ccco1] | 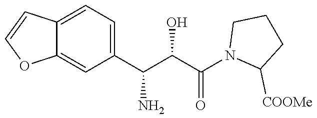 [SMILES: COC(=O)C1CCCN1C(=O)[C@@H](O)[C@H](N)c1ccc2ccoc2c1] |
| 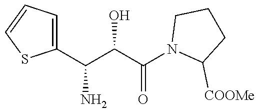 [SMILES: COC(=O)C1CCCN1C(=O)[C@@H](O)[C@H](N)c1cccs1] | |

In any embodiment herein, it may be that the compound is any of those listed Table 2, or a pharmaceutically acceptable salt and/or solvate thereof.

TABLE 2
C-Terminal Analogs

| | |
|---|---|
| 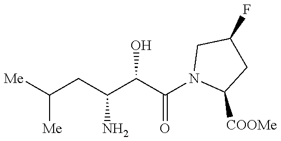 [SMILES: COC(=O)[C@@H]1C[C@H](F)CN1C(=O)[C@@H](O)[C@H](N)CC(C)C] | 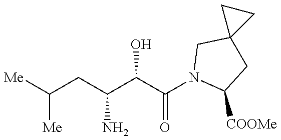 [SMILES: COC(=O)[C@@H]1CC2(CC2)CN1C(=O)[C@@H](O)[C@H](N)CC(C)C] |
| 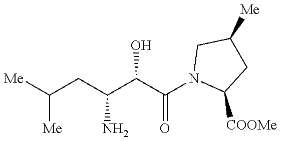 [SMILES: COC(=O)[C@@H]1C[C@H](C)CN1C(=O)[C@@H](O)[C@H](N)CC(C)C] | 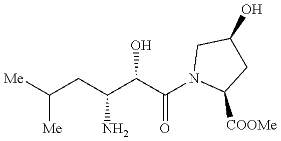 [SMILES: COC(=O)[C@@H]1C[C@H](O)CN1C(=O)[C@@H](O)[C@H](N)CC(C)C] |
| 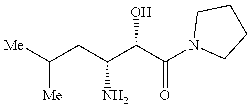 [SMILES: CC(C)C[C@@H](N)[C@H](O)C(=O)N1CCCC1] | 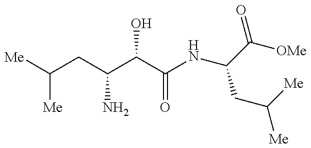 [SMILES: COC(=O)[C@H](CC(C)C)NC(=O)[C@@H](O)[C@H](N)CC(C)C] |
| 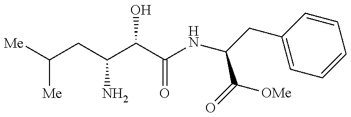 [SMILES: COC(=O)[C@H](Cc1ccccc1)NC(=O)[C@@H](O)[C@H](N)CC(C)C] | 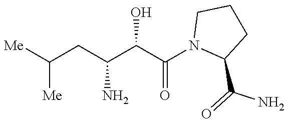 [SMILES: CC(C)C[C@@H](N)[C@H](O)C(=O)N1CCC[C@H]1C(N)=O] |
| 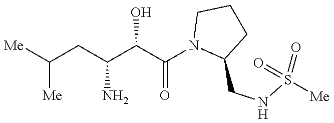 [SMILES: CC(C)C[C@@H](N)[C@H](O)C(=O)N1CCC[C@H]1CNS(C)(=O)=O] | 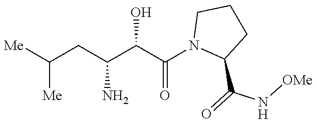 [SMILES: CONC(=O)[C@@H]1CCCN1C(=O)[C@@H](O)[C@H](N)CC(C)C] |
| 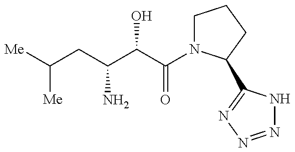 [SMILES: CC(C)C[C@@H](N)[C@H](O)C(=O)N1CCC[C@H]1c1nnn[nH]1] | 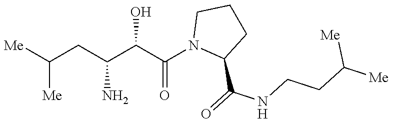 [SMILES: CC(C)CCNC(=O)[C@@H]1CCCN1C(=O)[C@@H](O)[C@H](N)CC(C)C] |

In any embodiment herein, it may be that the compound is any of those listed Table 3, or a pharmaceutically acceptable salt and/or solvate thereof.

TABLE 3
C-Terminal Peptide Analogs

| | |
|---|---|
| 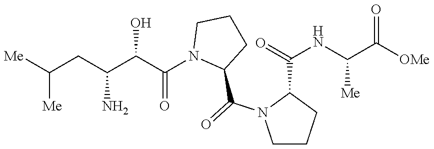 [SMILES: COC(=O)[C@H](C)NC(=O)[C@@H]1CCCN1C(=O)[C@@H]1CCCN1C(=O)[C@@H](O)[C@H](N)CC(C)C] | 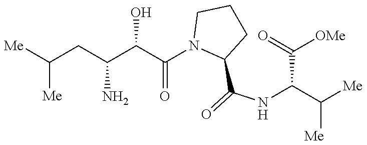 [SMILES: COC(=O)[C@@H](NC(=O)[C@@H]1CCCN1C(=O)[C@@H](O)[C@H](N)CC(C)C)C(C)C] |
| 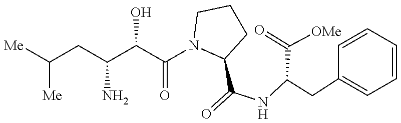 [SMILES: COC(=O)[C@H](Cc1ccccc1)NC(=O)[C@@H]1CCCN1C(=O)[C@@H](O)[C@H](N)CC(C)C] | 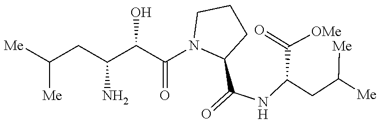 [SMILES: COC(=O)[C@H](CC(C)C)NC(=O)[C@@H]1CCCN1C(=O)[C@@H](O)[C@H](N)CC(C)C] |
| 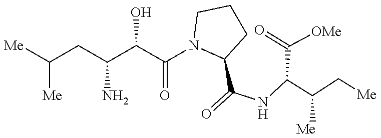 [SMILES: CC[C@H](C)[C@H](NC(=O)[C@@H]1CCCN1C(=O)[C@@H](O)[C@H](N)CC(C)C)C(=O)OC] | |

In any embodiment herein, it may be that the compound is any of those listed Table 3, or a pharmaceutically acceptable salt and/or solvate thereof.

TABLE 4
C-Terminal Amide Analogs

| | |
|---|---|
| 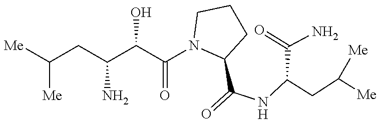 [SMILES: CC(C)C[C@H](NC(=O)[C@@H]1CCCN1C(=O)[C@@H](O)[C@H](N)CC(C)C)C(N)=O] | 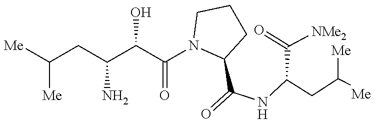 [SMILES: CC(C)C[C@H](NC(=O)[C@@H]1CCCN1C(=O)[C@@H](O)[C@H](N)CC(C)C)C(=O)N(C)C] |
| 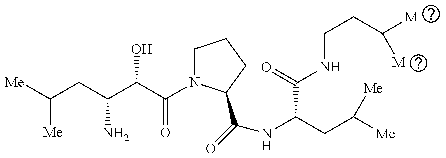 | 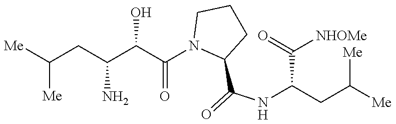 |
| 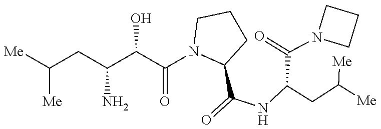 [SMILES: CC(C)C[C@H](NC(=O)[C@@H]1CCCN1C(=O)[C@@H](O)[C@H](N)CC(C)C)C(=O)N1CCC1] | 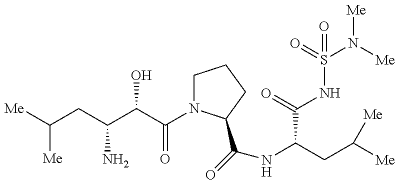 [SMILES: CC(C)C[C@H](NC(=O)[C@@H]1CCCN1C(=O)[C@@H](O)[C@H](N)CC(C)C)C(=O)NS(=O)(=O)N(C)C] |
| 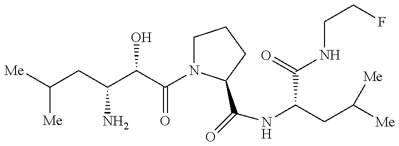 [SMILES: CC(C)C[C@H](NC(=O)[C@@H]1CCCN1C(=O)[C@@H](O)[C@H](N)CC(C)C)C(=O)NCCF] | 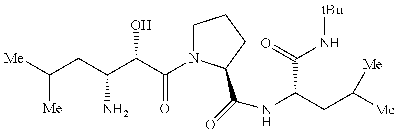 [SMILES: CC(C)C[C@H](NC(=O)[C@@H]1CCCN1C(=O)[C@@H](O)[C@H](N)CC(C)C)C(=O)NC(C)(C)C] |
| 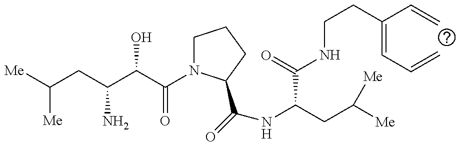 [SMILES: C=C/C=C(\C=C)CCNC(=O)[C@H](CC(C)C)NC(=O)[C@@H]1CCCN1C(=O)[C@@H](O)[C@H](N)CC(C)C] | 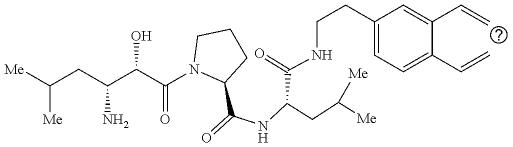 [SMILES: C=Cc1ccc(CCNC(=O)[C@H](CC(C)C)NC(=O)[C@@H]2CCCN2C(=O)[C@@H](O)[C@H](N)CC(C)C)cc1C=C] |
| 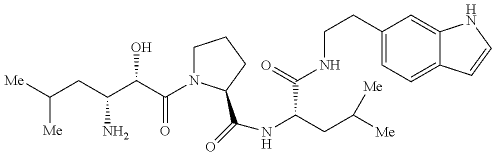 [SMILES: CC(C)C[C@H](NC(=O)[C@@H]1CCCN1C(=O)[C@@H](O)[C@H](N)CC(C)C)C(=O)NCCc1ccc2cc[nH]c2c1] | 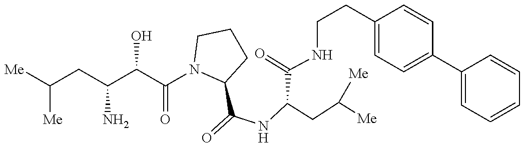 [SMILES: CC(C)C[C@H](NC(=O)[C@@H]1CCCN1C(=O)[C@@H](O)[C@H](N)CC(C)C)C(=O)NCCc1ccc(-c2ccccc2)cc1] |

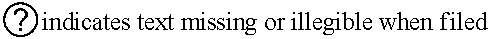

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology. The following Examples demonstrate the preparation, characterization, and use of illustrative compositions of the present technology that inhibit M24B aminopeptidases.

Synthesis of Compounds with Different N-Terminals

N-Terminal analogs were synthesized with a highly diastereoselective synthetic strategy using the chemistry of tert-butanesulfinamide (Scheme 1). The enolate addition of methyl 2-((tert-butoxycarbonyl)oxy)acetate to (S)-tert-butanesulfinylimines provided the enantiopure 9 with a (2S,3R) absolute stereochemistry in high yields (66%-87%). Notably, this methodology has been used to prepare the side chain of Taxol and has a wide substrate scope. Sequential hydrolysis of the methyl ester, coupling with proline methyl ester, and global deprotection afforded the N-terminal analogs (Table 1) of CQ31 in good overall yields (52%-76%). Moreover, C-terminal analogs (Tables 2-4) were synthesized by using intermediates of 11 and 12 with the indicated protecting groups to facilitate different deprotections (Scheme 2).

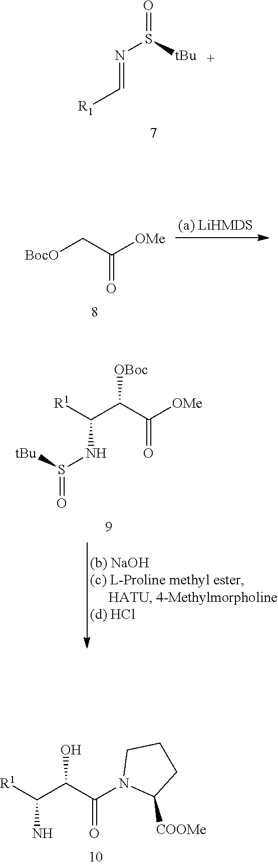

In Scheme 1, R¹ is isobutanyl, benzyl, 1,1,1,-trifluorobutanyl, 2,2,-dimethylbutanyl, isopropanyl, butanyl, 4-hydroxybutanyl, adamantanyl, cyclopropyl, phenyl, 2-furanyl, 6-benzofuranyl, or 2-thiophenyl. Reaction (a) was conducted with LiHMDS in THF at −78° C. Reaction (b) was conducted with NaOH, H₂O, and dioxane at 22° C. Reaction (c) was conducted with L-proline methyl ester, HATU (hexafluorophosphate azabenzotriazole tetramethyl uronium), 4-methylmorpholine, and CH₂Cl₂ at 0° C. Reaction (d) was conducted with HCl (3M in MeOH) at 22° C.

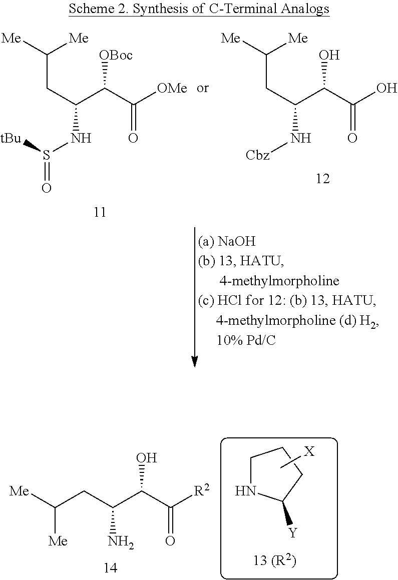

In Scheme 1, R² is methyl (2S,4S)-4-fluoropyrrolidine-2-carboxylate, methyl (S)-5-azasprio[2.4]heptane-6-carboxylate, methyl (2S,4S)methylpyrrolidine-2-carboxylate, pyrrolidine, methyl L-leucinate, methyl L-phenylalaninate, (S)-pyrrolidine-2-carboxyamide, (S)—N-(pyrrolidine-2-ylmethyl)methanesulfonamide, (S)—N-methoxypyrrolidine-2-carboxamide, (S)-5-(pyrrolidine-2-yl)-1H-tetrazole, (S)—N-isopentylpyrrolidine-2-carboxamide; methyl L-prolyl-L-prolyl-L-alaninate, methyl L-prolyl-L-valinate, methyl L-prolyl-L-phenylalaninate, methyl L-prolyl-L leucinate, methyl L-prolyl-L-isoleucinate, L-prolyl-L-leucinamide, dimethyl L-prolyl-L-leucinamide, isopentyl L-prolyl-L-leucinamide, N-methoxy L-prolyl-L-leucinamide, L-prolyl-L-leucyl-azetidin, N,N-dimethylsulfamoyl L-prolyl-L-leucinamide, 2-fluoroethyl L-prolyl-L-leucinamide, tert-butyl L-prolyl-L-leucinamide, phenethyl L-prolyl-L-leucinamide, 2-(naphthalen-2-yl) L-prolyl-L-leucinamide, 2-(1H-indol-5-yl)ethyl L-prolyl-L-leucinamide, or 2-([1,1′-biphenyl]-4-yl)ethyl L-prolyl-L-leucinamide. Reaction (a) was conducted with NaOH, H₂O, and dioxane at 22° C. Reaction (b) was conducted with 13, HATU, 4-methylmorpholine, and CH₂Cl₂ at 0° C. Reaction (c) was conducted with HCl (3 M in MeOH) at 22° C. Reaction (d) was conducted with 10% Pd/C in MeOH at 22° C.

### Exemplary Synthetic Procedures

Materials and Methods. All reactions were carried out under an argon atmosphere with dry solvents under anhydrous conditions, unless otherwise noted. Chemical reagents were purchased from Aldrich, Acros, or Fisher at the highest commercial quality and used without further purification, unless otherwise stated. Other reagents used for assays include Val-boroPro (VbP; Tocris 3719), bestatin methyl ester (MeBs; Sigma, 200485), apstatin (SCBT, sc-201309), dTAG-13 (R&D Systems, 6605/5), and FuGENE HD (Promega, E2311). Reactions were monitored by thin layer chromatography (TLC) carried out on MilliporeSigma glass TLC plates (silica gel 60 coated with F254, 250 μm) using UV light for visualization and aqueous ammonium cerium nitrate/ammonium molybdate or basic aqueous potassium permanganate as developing agent. NMR spectra were recorded on a Bruker Ultrashield Plus Avance III 500 MHz or Bruker Avance III 600 MHz NMR. The spectra were calibrated by using residual undeuterated solvents (for 1H NMR) and deuterated solvents (for 13C NMR) as internal references: undeuterated chloroform (δH=7.26 ppm) and CDCl3 (δC=77.16 ppm); undeuterated methanol (δH=3.31 ppm) and methanol-d4 (δC=49.00 ppm). The following abbreviations are used to designate multiplicities: s=singlet, d=doublet, t=triplet, q=quartet, m=multiplet, br=broad. High-resolution mass spectra (HRMS) were recorded on a Waters Micromass LCT Premier XE TOF LC-MS. Purity of the compounds were assayed using a Waters Acquity ultraperformance liquid chromatography (UPLC) system equipped with a SQ Detector and an ELS Detector. Gradient solvent consisted of 0.1% TFA and 5-95% acetonitrile in water over 8 min maintaining a constant flow rate of 0.30 ml/min, and all final compounds were ≥95%.

General Synthetic Methods. Diastereoselective enolate addition of methyl 2-((tert-butoxycarbonyl)oxy)acetate to (S)-tert-butanesulfinylimines (Method A): A solution of methyl 2-((tert-butoxycarbonyl)oxy)acetate 8 (951 mg, 5.0 mmol) in dry THF (15 mL) maintained under an atmosphere of argon was cooled to −78° C. and then treated with LiHMDS (5.0 mL, 1.0 M solution in THF, 5.0 mmol). The reaction mixture was stirred for 1 hour (h) at the same temperature before imine 7 (1.0 mmol) in THF (1.0 mL) was added slowly. The mixture was allowed to stir for 5 h before it was quenched with saturated aq. NH₄Cl (30 mL). The aqueous phase was extracted with EtOAc (3×30 mL). The combined organic phases were washed with brine (50 mL), dried over anhydrous MgSO₄, filtered and concentrated under vacuum. The residue was passed through a short plug of silica gel with EtOAc:hexane=1:6, v/v→1:1, v/v) to give the desired methyl ester 9 as a colorless oil, white power or solid.

Hydrolysis of the methyl ester, coupling with proline methyl ester and global deprotection (Method B): To the solution of methyl ester 9 (1.0 mmol) in 1,4-dioxane/H₂O (1:1, 100 mL was added NaOH (60 mg, 1.5 mmol) and the reaction mixture was stirred at 22° C. for 1 h. The mixture was acidified to pH 3-4 with Dowex® 50W X8 resin. The resin was filtered and washed with CH₂Cl₂. The aqueous phase was extracted with CH₂Cl₂ (3×100 mL). The combined organic phases were washed with brine (100 mL), dried over anhydrous MgSO₄, filtered and concentrated under vacuum to give a colorless oil which was used for the next step without further purifications. To a solution of crude oil from the last step in CH₂Cl₂ (20 mL) were sequentially added L-Proline methyl ester hydrochloride (199 mg, 1.2 mmol), HATU (457 mg, 1.2 mmol), 4-Methylmorpholine (253 mg, 275 μL, 2.5 mmol) at 0° C. The reaction mixture was allowed to stir for another 3 h before it was quenched by addition of saturated aq. NaHCO₃ solution (10 mL). The organic layer was separated, and the aqueous layer was extracted with CH₂Cl₂ (3×20 mL). The organic layers were combined, washed with brine (50 mL), dried over Na₂SO₄, and concentrated under vacuum. The resulting residue was purified by flash column chromatography (silica gel, acetone:hexane=1:4, v/v→1:1, v/v) to give the desired amide as a colorless oil. To a stirred solution of the obtained oil in MeOH (30 mL) was added HCl (5 mL, 3.0 M solution in MeOH, 15 mmol) at 0° C. The reaction mixture was warmed to 22° C. and stirred for 24 h at the same temperature. The mixture was concentrated under vacuum, and the residue was purified by recrystallization from MeOH/diethyl ether to give 10 as a white solid.

Coupling with proline methyl ester and Cbz deprotection (Method C): To a solution of carboxylic acid 12 (295 mg, 1.0 mmol) in CH₂Cl₂ (30 mL) were sequentially added L-Proline analogs 13 (1.2 mmol), HATU (457 mg, 1.2 mmol), 4-Methylmorpholine (253 mg, 275 μL, 2.5 mmol) at 0° C. The reaction mixture was allowed to stir for another 4 h before it was quenched by addition of saturated aqueous NaHCO₃ solution (10 mL). The organic layer was separated, and the aqueous layer was extracted with CH₂Cl₂ (3×30 mL). The organic layers were combined, washed with brine (30 mL), dried over Na₂SO₄, and concentrated under vacuum. The resulting residue was purified by flash column chromatography (silica gel, acetone:hexanes=1:4, v/v→1:1, v/v) to give the desired amide as a colorless oil. To a stirred solution of the obtained oil in MeOH (20 mL) was added sequentially AcOH (50 uL) and Pd/C (53.2 mg, 0.05 mmol, 10 wt. %) at 22° C. The resultant mixture was stirred under H₂ (1 atm) at that temperature for 2 h before it was diluted with EtOAc (30 mL) and passed through a plug of Celite. To the filtrate was added HCl (500 μL, 2.0 M in Et₂O, 1.0 mmol) and the solvent was removed under vacuum. The residue was purified by recrystallization from MeOH/diethyl ether to give 14 as a white solid.

Methyl (2S,3R)-2-((tert-butoxycarbonyl)oxy)-3-(((S)-tert-butylsulfinyl)amino)-4-phenylbutanoate (9b; Method A): White powder; yield 72%; ¹H NMR (600 MHz, Chloroform-d) δ 7.31-7.20 (m, 5H), 4.23 (dd, J=10.5, 6.9 Hz, 1H), 4.02 (dt, J=8.6, 6.8 Hz, 1H), 3.63 (s, 3H), 3.50 (dd, J=14.0, 8.4 Hz, 1H), 3.24 (dd, J=13.9, 6.6 Hz, 1H), 1.57 (s, 9H), 1.06 (s, 9H). ¹³C NMR (151 MHz, CDCl₃) δ 173.52, 155.36, 137.52, 129.84, 128.72, 127.09, 84.17, 71.57, 60.21, 53.77, 52.18, 35.81, 28.44, 22.70. HRMS (m/z): [M+Na]⁺ calculated for C₂₀H₃₁NO₆NaS⁺ 436.1770, found 436.1769.

Methyl (2S,3R)-2-((tert-butoxycarbonyl)oxy)-3-(((S)-tert-butylsulfinyl)amino)-6,6,6-trifluorohexanoate (9c; Method A): White powder; yield 75%; ¹H NMR (600 MHz, Chloroform-d) δ 5.01 (d, J=2.6 Hz, 1H), 3.85 (ddd, J=10.9, 8.5, 5.9 Hz, 1H), 3.75 (s, 3H), 3.42 (d, J=10.4 Hz, 1H), 2.55-2.45 (m, 1H), 2.30-2.20 (m, 1H), 1.99 (dddd, J=14.1, 10.7, 8.5, 5.4 Hz, 1H), 1.96-1.86 (m, 1H), 1.51 (s, 9H), 1.17 (s, 9H). ¹³C NMR (151 MHz, CDCl₃) δ 168.19, 152.63, 129.65, 127.82, 125.99, 124.16, 84.14, 76.36, 58.02, 56.72, 52.65, 31.03, 30.84, 30.64, 30.45, 27.76, 26.50, 26.49, 26.47, 26.45, 22.68. HRMS (m/z): [M+Na]⁺ calculated for C₁₆H₂₈NO₆F₃NaS⁺ 442.1487, found 442.1486.

Methyl (2S,3R)-2-((tert-butoxycarbonyl)oxy)-3-(((S)-tert-butylsulfinyl)amino)-5,5-dimethylhexanoate (9d; Method A): White powder; yield 82%; ¹H NMR (600 MHz, Chloroform-d) δ 5.62 (s, 1H), 4.31 (dd, J=11.2, 4.4 Hz, 1H), 4.01 (d, J=11.8 Hz, 1H), 3.73 (s, 3H), 2.79 (t, J=13.1 Hz, 1H), 1.53 (s, 9H), 1.40 (d, J=14.2 Hz, 1H), 1.20 (s, 9H), 1.02 (s, 9H). ¹³C NMR (151 MHz, CDCl₃) δ 173.54, 156.75, 84.48, 73.87, 60.82, 52.09, 48.68, 42.12, 30.52, 30.41, 28.42, 23.38. HRMS (m/z): [M+Na]⁺ calculated for C₁₈H₃₅NO₆NaS⁺ 416.2083, found 416.2090.

Methyl (2S,3R)-2-((tert-butoxycarbonyl)oxy)-3-(((S)-tert-butylsulfinyl)amino)-4-methylpentanoate (9e; Method A): White powder; yield 70%; ¹H NMR (600 MHz, Chloroform-d) δ 4.62 (s, 1H), 4.33 (t, J=7.9 Hz, 1H), 3.75 (s, 3H), 3.52 (dd, J=9.1, 6.4 Hz, 1H), 2.75-2.52 (m, 1H), 1.52 (s, 9H), 1.25 (s, 9H), 1.06 (d, J=7.0 Hz, 3H), 1.02 (d, J=6.7 Hz, 3H). ¹³C NMR (151 MHz, CDCl₃) δ 174.07, 155.79, 83.84, 76.95, 72.10, 60.32, 58.12, 52.29, 29.05, 28.36, 22.90, 21.78, 21.60. HRMS (m/z): [M+H]⁺ calculated for C₂₀H₃₁NO₆S⁺ 388.1762, found 388.1770.

Methyl (2S,3R)-2-((tert-butoxycarbonyl)oxy)-3-(((S)-tert-butylsulfinyl)amino)hept-6-enoate (9f; Method A): White powder; yield 77%; ¹H NMR (600 MHz, Chloroform-d) δ 5.77 (ddd, J=16.6, 10.4, 7.3 Hz, 1H), 5.08-4.95 (m, 2H), 4.31 (d, J=6.4 Hz, 1H), 3.77 (dd, J=10.0, 4.8, 4.3 Hz, 1H), 3.73 (s, 3H), 2.53 (s, 1H), 2.23-2.13 (m, 1H), 2.11-1.99 (m, 1H), 1.71 (dd, J=9.4, 5.7 Hz, 1H), 1.50 (s, 9H), 1.21 (s, 9H). ¹³C NMR (151 MHz, CDCl₃) δ 173.31, 155.22, 137.01, 115.77, 84.08, 71.86, 60.15, 52.06, 51.05, 31.29, 29.67, 28.24, 22.82. HRMS (m/z): [M+Na]⁺ calculated for C₁₇H₃₁NO₆NaS⁺ 400.1770, found 400.1780.

Methyl (2S,3R)-3-(adamantan-1-yl)-2-((tert-butoxycarbonyl)oxy)-3-(((S)-tert-butylsulfinyl)-amino)propanoate (9h; Method A): White powder; yield 66%; ¹H NMR (600 MHz, Chloroform-d) δ 5.30 (br.s, 1H), 3.70 (s, 3H), 3.67-3.63 (m, 1H), 3.42-3.38 (m, 1H), 1.85-1.80 (m, 3H), 2.04-1.99 (m, 3H), 1.71-1.60 (m, 6H), 1.54-1.50 (m, 3H), 1.49 (s, 9H), 1.16 (s, 9H). ¹³C NMR (151 MHz, CDCl₃) δ 169.75, 152.66, 83.37, 74.36, 67.32, 56.93, 52.46, 39.53, 36.77, 36.58, 28.56, 27.85, 22.84. HRMS (m/z): [M+Na]⁺ calculated for C₂₃H₃₉NO₆NaS⁺ 480.2404, found 480.2396.

Methyl (2S,3R)-2-((tert-butoxycarbonyl)oxy)-3-(((S)-tert-butylsulfinyl)amino)-3-cyclopropyl-propanoate (9i; Method A): White powder; yield 75%; ¹H NMR (600 MHz, Chloroform-d) δ 5.09 (d, J=2.9 Hz, 1H), 3.72 (s, 3H), 3.57 (d, J=9.8 Hz, 1H), 2.92 (dd, J=9.8, 3.0 Hz, 1H), 1.50 (s, 9H), 1.16 (s, 9H), 1.15-1.10 (m, 1H), 0.81-0.74 (m, 1H), 0.68-0.60 (m, 2H), 0.43-0.34 (m, 1H). ¹³C NMR (151 MHz, CDCl₃) δ 168.56, 152.86, 83.59, 77.37, 64.31, 56.47, 52.43, 27.81, 22.65, 14.80, 6.00, 4.93. HRMS (m/z): [M+Na]⁺ calculated for C₁₆H₂₉NO₆NaS⁺ 386.1613, found 386.1628.

Methyl (2S,3R)-2-((tert-butoxycarbonyl)oxy)-3-(((S)-tert-butylsulfinyl)amino)-3-phenylprop-anoate (9j; Method A): White powder; yield 87%; ¹H NMR (600 MHz, Chloroform-d) δ 7.41 (d, J=7.4 Hz, 2H), 7.36 (t, J=7.6 Hz, 2H), 7.29 (t, J=7.2 Hz, 1H), 5.24 (d, J=2.9 Hz, 1H), 4.99 (dd, J=9.9, 2.9 Hz, 1H), 4.15 (d, J=9.8 Hz, 1H), 3.77 (s, 3H), 1.41 (s, 9H), 1.18 (s, 9H). ¹³C NMR (151 MHz, CDCl₃) δ 168.40, 152.43, 138.15, 128.80, 128.30, 127.34, 83.69, 77.83, 60.55, 56.92, 52.71, 27.72, 22.61. HRMS (m/z): [M+Na]⁺ calculated for C₁₉H₂₉NO₆NaS⁺ 422.1612, found 422.1613.

Methyl (2S,3S)-2-((tert-butoxycarbonyl)oxy)-3-(((S)-tert-butylsulfinyl)amino)-3-(furan-2-yl)-propanoate (9k; Method A): White powder; yield 84%; ¹H NMR (600 MHz, Chloroform-d) δ 7.38 (dd, J=2.0, 0.9 Hz, 1H), 6.51 (dt, J=3.3, 1.0 Hz, 1H), 6.33 (dd, J=3.3, 1.8 Hz, 1H), 5.48 (d, J=2.9 Hz, 1H), 5.02 (ddd, J=10.7, 2.9, 1.0 Hz, 1H), 3.90 (d, J=10.7 Hz, 1H), 3.78 (s, 3H), 1.46 (s, 9H), 1.20 (s, 9H). ¹³C NMR (151 MHz, CDCl₃) δ 168.04, 152.40, 151.15, 142.82, 110.82, 109.34, 83.76, 75.65, 56.97, 56.41, 52.76, 27.76, 22.58. HRMS (m/z): [M+Na]⁺ calculated for C₁₇H₂₇NO₇NaS⁺ 412.1421, found 412.1406.

Methyl (2S,3R)-3-(benzofuran-6-yl)-2-((tert-butoxycarbonyl)oxy)-3-(((S)-tert-butylsulfinyl)-amino)propanoate (9l; Method A): White powder; yield 72%; ¹H NMR (600 MHz, Chloroform-d) δ 7.60 (d, J=1.8 Hz, 1H), 7.55 (d, J=2.2 Hz, 1H), 7.41 (d, J=8.5 Hz, 1H), 7.26 (dd, J=8.6, 2.0 Hz, 1H), 6.70 (dd, J=2.2, 0.9 Hz, 1H), 5.19 (d, J=3.1 Hz, 1H), 5.02 (dd, J=9.8, 3.0 Hz, 1H), 4.11 (d, J=9.8 Hz, 1H), 3.70 (s, 3H), 1.32 (s, 9H), 1.12 (s, 9H). ¹³C NMR (151 MHz, CDCl₃) δ 168.44, 154.78, 152.43, 145.74, 132.86, 127.82, 123.61, 120.38, 111.67, 106.96, 83.70, 78.14, 60.68, 56.92, 52.71, 27.71, 22.61. HRMS (m/z): [M+Na]⁺ calculated for C₂₁H₂₉NO₇NaS⁺ 462.1562, found 462.1560.

Methyl (2S,3S)-2-((tert-butoxycarbonyl)oxy)-3-(((S)-tert-butylsulfinyl)amino)-3-(thiophen-2-yl)propanoate (9m; Method A): White powder; yield 75%; ¹H NMR (600 MHz, Chloroform-d) δ 7.41 (dt, J=2.5, 1.1 Hz, 1H), 7.31 (dd, J=5.1, 3.0 Hz, 1H), 7.13 (dd, J=5.1, 1.4 Hz, 1H), 5.37 (d, J=2.8 Hz, 1H), 5.08 (ddd, J=10.1, 2.8, 1.0 Hz, 1H), 3.93 (d, J=10.1 Hz, 1H), 3.77 (s, 3H), 1.45 (s, 9H), 1.19 (s, 9H). ¹³C NMR (151 MHz, CDCl₃) δ 168.29, 152.59, 139.47, 126.67, 126.49, 123.70, 83.80, 76.95, 57.92, 56.87, 52.71, 27.75, 22.65. HRMS (m/z): [M+Na]⁺ calculated for C₁₇H₂₇NO₆NaS₂⁺ 428.1178, found 428.1193.

Methyl ((2S,3R)-3-amino-2-hydroxy-4-phenylbutanoyl)-L-prolinate (10b; Method B): White powder; yield 57%; HPLC purity: 95.81%; ¹H NMR (600 MHz, Methanol-d₄) δ 7.45-7.18 (m, 5H), 4.93 (d, J=8.1 Hz, 0.17H), 4.49 (dd, J=20.4, 9.3 Hz, 0.17H), 4.40 (br.s, 0.83H), 4.28 (d, J=9.0 Hz, 0.83H), 3.85-3.76 (m, 1H), 3.70 (s, 3H), 3.29-2.74 (m, 4H), 2.35-1.74 (m, 4H). ¹³C NMR (151 MHz, MeOD, more than 16 ¹³C signals for compound 10b were observed due to the presence of different rotameric species) δ 174.04, 173.92, 170.93, 170.51, 136.75, 130.35, 130.16, 130.10, 130.07, 128.59, 128.49, 69.66, 68.67, 60.56, 60.39, 57.83, 56.35, 53.32, 52.86, 49.57, 48.27, 48.10, 38.91, 35.65, 35.24, 32.14, 29.73, 25.71, 22.73. HRMS (m/z): [M+H]⁺ calculated for C₁₆H₂₃N₂O₄⁺ 307.1658, found 307.1673.

Methyl ((2S,3R)-3-amino-6,6,6-trifluoro-2-hydroxyhexanoyl)-L-prolinate (10c; Method B): White solid; yield 55%; HPLC purity: 98.71%; ¹H NMR (600 MHz, Methanol-d₄) δ 4.78 (d, J=8.6 Hz, 0.19H), 4.51 (d, J=3.7 Hz, 0.81H), 4.49 (dd, J=8.7, 4.3 Hz, 1H), 3.83 (dd, J=10.0, 7.2 Hz, 0.81H), 3.77-3.74 (m, 0.81H), 3.74 (s, 0.57H), 3.72 (s, 2.43H), 3.71-3.62 (m, 0.38H), 3.62-3.57 (m, 0.19H), 3.55 (dd, J=6.9, 3.5 Hz, 0.81H), 2.47-2.33 (m, 2H), 2.34-2.24 (m, 1H), 2.19-1.86 (m, 5H). ¹³C NMR (151 MHz, MeOD, more than 12 ¹³C signals for compound 10c were observed due to the presence of different rotameric species) δ 175.04, 174.11, 171.23, 130.95, 129.11, 127.29, 125.46, 69.79, 68.41, 61.30, 60.69, 53.67, 53.58, 52.93, 49.57, 48.49, 48.39, 32.20, 31.02, 30.82, 30.63, 30.43, 29.95, 25.84, 23.46, 22.44. HRMS (m/z): [M+H]⁺ calculated for C₁₂H₂₀N₂O₄F₃⁺ 313.1375, found 313.1373.

Methyl ((2S,3R)-3-amino-2-hydroxy-5,5-dimethylhexanoyl)-L-prolinate (10d; Method B): White solid; yield 67%; HPLC purity: 96.23%; ¹H NMR (600 MHz, Methanol-d₄) δ 4.75 (dd, J=8.7, 1.8 Hz, 0.14H), 4.49 (dd, J=8.7, 4.5 Hz, 0.86H), 4.42 (d, J=3.9 Hz, 0.86H), 4.39 (d, J=2.2 Hz, 0.14H), 3.87-3.81 (m, 1H), 3.74 (s, 0.42H), 3.73 (s, 2.58H), 3.69-3.62 (m, 1H), 3.62-3.57 (m, 0.14H), 3.55 (dd, J=5.4, 3.9 Hz, 0.86H), 2.35-2.23 (m, 1H), 2.17-1.86 (m, 3H), 1.81 (dd, J=14.9, 5.4 Hz, 0.86H), 1.78 (dd, J=14.9, 6.4 Hz, 0.14H), 1.46 (dd, J=15.0, 5.5 Hz, 0.86H), 1.42 (dd, J=15.4, 5.1 Hz, 0.14H), 1.02 (s, 7.74H), 1.01 (s, 1.26H). ¹³C NMR (151 MHz, MeOD, more than 14 ¹³C signals for compound 10d were observed due to the presence of different rotameric species) δ 175.25, 174.24, 171.63, 171.17, 72.29, 70.20, 61.41, 60.64, 52.98, 52.97, 52.00, 51.75, 49.57, 48.51, 43.97, 43.76, 31.19, 31.15, 29.94, 29.82, 29.80, 25.85, 22.44. HRMS (m/z): [M+H]⁺ calculated for C₁₄H₂₇N₂O₄⁺ 287.1971, found 287.1972.

Methyl ((2S,3R)-3-amino-2-hydroxy-4-methylpentanoyl)-L-prolinate (10e; Method B): White solid; yield 65%; HPLC purity: 96.80%; ¹H NMR (600 MHz, Methanol-d₄) δ 4.79 (d, J=7.8 Hz, 0.17H), 4.63 (d, J=3.2 Hz, 0.83H), 4.59 (br.s, 0.17H), 4.50 (dd, J=8.7, 4.6 Hz, 0.83H), 3.90-3.83 (m, 0.83H), 3.75 (s, 0.51H), 3.73 (s, 2.49H), 3.74-3.69 (m, 0.83H), 3.67-3.53 (m, 0.34H), 3.33 (br.s, 0.17H), 3.19 (dd, J=7.8, 3.1 Hz, 0.83H), 2.47-2.37 (m, 0.17H), 2.35-2.25 (m, 0.83H), 2.25-1.86 (m, 4H), 1.14 (d, J=6.6 Hz, 0.51H), 1.10 (d, J=6.7 Hz, 2.49H), 1.09 (d, J=6.7 Hz, 2.49H), 1.06 (d, J=6.7 Hz, 0.51H). ¹³C NMR (151 MHz, MeOD, more than 12 ¹³C signals for compound 10e were observed due to the presence of different rotameric species) δ 175.23, 174.10, 171.97, 171.55, 68.83, 67.08, 61.31, 60.67, 60.18, 60.11, 53.09, 52.97, 48.44, 46.72, 32.28, 29.93, 28.88, 28.78, 25.91, 22.49, 19.57, 19.15, 18.65. HRMS (m/z): [M+H]⁺ calculated for C₁₂H₂₃N₂O₄⁺ 259.1658, found 259.1654.

Methyl ((2S,3R)-3-(adamantan-1-yl)-3-amino-2-hydroxypropanoyl)-L-prolinate (10h; Method B): White solid; yield 72%; HPLC purity: 95.74%; ¹H NMR (600 MHz, Methanol-d₄) δ 4.80 (d, J=1.8 Hz, 0.90H), 4.76 (d, J=9.3 Hz, 0.10H), 4.69 (br.s, 0.10H), 4.50 (dd, J=8.7, 4.8 Hz, 0.90H), 3.86-3.79 (m, 0.90H), 3.78 (s, 0.30H), 3.73 (s, 2.70H), 3.71-3.65 (m, 0.90H), 3.65-3.54 (m, 0.20H), 3.35 (br.s, 0.10H), 3.06 (br.s, 0.90H), 2.45-2.37 (m, 0.10H), 2.34-2.25 (m, 1H), 2.26-2.17 (m, 0.20H), 2.12-2.02 (m, 5.90H), 2.03-1.96 (m, 1.80H), 1.85-1.63 (m, 10H). ¹³C NMR (151 MHz, MeOD) δ 174.20, 172.55, 64.91, 62.62, 60.73, 52.97, 49.85, 49.57, 39.43, 39.09, 37.49, 37.33, 29.86, 29.55, 25.93. HRMS (m/z): [M+H]⁺ calculated for C₁₉H₃₁N₂O₄⁺ 351.2284, found 351.2291.

Methyl ((2S,3R)-3-amino-3-cyclopropyl-2-hydroxypropanoyl)-L-prolinate (10i; Method B): White solid; yield 76%; HPLC purity: 98.96%; ¹H NMR (500 MHz, Methanol-d₄) δ 4.79 (d, J=8.2 Hz, 0.19H), 4.58 (d, J=4.5 Hz, 0.81H), 4.51 (d, J=3.6 Hz, 0.19H), 4.48 (dd, J=8.7, 4.3 Hz, 0.81H), 3.89-3.81 (m, 0.81H), 3.76 (s, 0.57H), 3.80-3.73 (m, 0.81H), 3.72 (s, 2.43H), 3.70-3.52 (m, 0.38H), 2.72 (dd, J=10.8, 3.7 Hz, 0.19H), 2.65 (dd, J=10.5, 4.4 Hz, 0.81H), 2.34-2.23 (m, 0.81H), 2.23-2.15 (m, 0.19H), 2.13-1.87 (m, 3H), 1.20-1.07 (m, 1H), 0.80-0.64 (m, 2H), 0.55-0.35 (m, 2H). ¹³C NMR (126 MHz, MeOD, more than 12 ¹³C signals for compound 10i were observed due to the presence of different rotameric species) δ 174.76, 174.09, 171.59, 171.33, 71.51, 70.21, 61.17, 60.71, 60.62, 60.23, 53.08, 52.86, 48.42, 32.20, 29.98, 25.86, 22.64, 11.71, 11.62, 4.94, 4.67, 4.64, 4.30. HRMS (m/z): [M+H]⁺ calculated for C₁₂H₂₁N₂O₄⁺ 257.1501, found 257.1497.

Methyl ((2S,3R)-3-amino-2-hydroxy-3-phenylpropanoyl)-L-prolinate (10j; Method B): White solid; yield 67%; HPLC purity: 98.99%; ¹H NMR (600 MHz, Methanol-d₄) δ 7.54-7.43 (m, 5H), 4.69 (d, J=6.4 Hz, 0.81H), 4.56 (d, J=6.4 Hz, 0.19H), 4.54 (d, J=6.6 Hz, 0.81H), 4.52 (d, J=6.7 Hz, 0.19H), 3.73 (s, 0.57H), 3.74-3.67 (m, 0.81H), 3.61 (s, 2.43H), 3.56-3.50 (m, 0.19H), 3.39-3.33 (m, 0.19H), 3.30-3.23 (m, 0.81H), 2.27-2.14 (m, 0.81H), 2.04-1.98 (m, 0.19H), 1.97-1.84 (m, 3.43H), 1.82-1.70 (m, 0.57H). ¹³C NMR (151 MHz, MeOD, more than 15 ¹³C signals for compound 10j were observed due to the presence of different rotameric species) δ 173.86, 173.69, 170.96, 170.89, 135.08, 134.95, 130.69, 130.57, 130.35, 130.24, 129.10, 128.62, 72.81, 71.51, 60.76, 60.61, 59.26, 58.47, 53.23, 52.79, 48.26, 47.93, 31.81, 29.94, 25.77, 22.68. HRMS (m/z): [M+H]⁺ calculated for C₁₅H₂₁N₂O₄⁺ 293.1501, found 293.1496.

Methyl ((2S,3S)-3-amino-3-(furan-2-yl)-2-hydroxypropanoyl)-L-prolinate (10k; Method B): White solid; yield 58%; HPLC purity: 95.70%; ¹H NMR (600 MHz, Methanol-d₄) δ 7.65 (dd, J=1.9, 0.8 Hz, 0.18H), 7.64 (dd, J=1.9, 0.8 Hz, 0.82H), 6.60 (d, J=3.4 Hz, 0.82H), 6.56 (d, J=3.5 Hz, 0.18H), 6.51 (dd, J=3.4, 1.9 Hz, 1H), 4.79 (d, J=7.0 Hz, 0.82H), 4.72 (d, J=6.5 Hz, 0.18H), 4.67 (d, J=7.1 Hz, 0.82H), 4.65 (d, J=7.1 Hz, 0.18H), 4.43 (dd, J=8.8, 4.5 Hz, 0.82H), 4.39 (dd, J=8.3, 2.3 Hz, 0.18H), 3.80-3.76 (m, 0.82H), 3.75 (s, 0.54H), 3.65 (s, 2.46H), 3.62-3.55 (m, 0.18H), 3.49-3.40 (m, 1H), 2.29-2.20 (m, 0.82H), 2.16-2.06 (m, 0.36H), 2.03-1.90 (m, 2.46H), 1.90-1.82 (m, 0.36H). ¹³C NMR (151 MHz, MeOD, more than 13 ¹³C signals for compound 10k were observed due to the presence of different rotameric species) δ 174.16, 173.65, 170.52, 170.37, 147.89, 145.32, 145.21, 112.25, 112.16, 111.90, 111.51, 71.15, 69.71, 60.73, 53.15, 52.76, 52.11, 49.57, 48.19, 48.12, 32.07, 29.98, 25.80, 22.72. HRMS (m/z): [M+H]⁺ calculated for C₁₃H₁₉N₂O₅⁺ 283.1294, found 283.1296.

Methyl ((2S,3R)-3-amino-3-(benzofuran-6-yl)-2-hydroxypropanoyl)-L-prolinate (10l; Method B): White solid; yield 57%; HPLC purity: 98.27%; ¹H NMR (600 MHz, Methanol-d₄) δ 7.86 (d, J=2.2 Hz, 0.18H), 7.84 (d, J=2.2 Hz, 0.82H), 7.81 (d, J=1.9 Hz, 0.82H), 7.77 (d, J=1.8 Hz, 0.18H), 7.62 (d, J=8.6 Hz, 0.18H), 7.61 (d, J=8.5 Hz, 0.82H), 7.47 (dd, J=8.6, 1.9 Hz, 0.82H), 7.42 (dd, J=8.5, 1.8 Hz, 0.18H), 6.93 (d, J=1.3 Hz, 0.82H), 6.91 (d, J=1.4 Hz, 0.18H), 4.76 (d, J=6.9 Hz, 0.82H), 4.66 (d, J=6.9 Hz, 0.18H), 4.64 (d, J=6.9 Hz, 0.82H), 4.57 (d, J=6.9 Hz, 0.18H), 4.38 (dd, J=8.7, 4.2 Hz, 0.82H), 4.12 (br.d, J=8.6 Hz, 0.18H), 3.73 (s, 0.54H), 3.73-3.63 (m, 1H), 3.62-3.57 (m, 0.18H), 3.50 (s, 2.46H), 3.27-3.20 (m, 0.82H), 2.23-2.14 (m, 0.82H), 2.01-1.80 (m, 2.64H), 1.78-1.61 (m, 0.54H). ¹³C NMR (151 MHz, MeOD, more than 17 ¹³C signals for compound 10l were observed due to the presence of different rotameric species) δ 173.86, 173.60, 171.07, 170.93, 156.66, 156.58, 148.19, 147.89, 129.63, 129.56, 129.54, 125.16, 124.72, 122.53, 121.80, 112.98, 112.89, 107.81, 107.64, 73.00, 71.73, 60.72, 60.57, 59.34, 58.69, 53.22, 52.66, 48.26, 47.89, 31.69, 29.91, 25.76, 25.72, 22.64. HRMS (m/z): [M+H]⁺ calculated for C₁₇H₂₁N₂O₅⁺ 333.1450, found 333.1442.

Methyl ((2S,3S)-3-amino-2-hydroxy-3-(thiophen-2-yl)propanoyl)-L-prolinate (10m; Method B): White solid; yield 52%; HPLC purity: 96.00%; ¹H NMR (600 MHz, Methanol-d₄) δ 7.63 (dd, J=3.1, 1.3 Hz, 0.82H), 7.61 (dd, J=3.0, 1.3 Hz, 0.18H), 7.57 (dd, J=5.0, 2.8 Hz, 0.18H), 7.56 (dd, J=5.0, 2.9 Hz, 0.82H), 7.30 (dd, J=5.1, 1.3 Hz, 0.82H), 7.23 (dd, J=5.1, 1.3 Hz, 0.18H), 4.71 (d, J=6.2 Hz, 0.18H), 4.69 (d, J=6.3 Hz, 0.82H), 4.67 (d, J=6.3 Hz, 0.82H), 4.52 (d, J=6.5 Hz, 0.18H), 4.42 (dd, J=8.7, 4.2 Hz, 0.82H), 4.24 (dd, J=8.5, 2.0 Hz, 0.18H), 3.74 (s, 0.54H), 3.76-3.71 (m, 1.82H), 3.66 (s, 2.46H), 3.61-3.53 (m, 0.18H), 3.44-3.39 (m, 0.18H), 3.38-3.35 (m, 0.82H), 2.28-2.19 (m, 0.82H), 2.10-2.05 (m, 0.18H), 2.01-1.88 (m, 2.64H), 1.87-1.77 (m, 0.36H). ¹³C NMR (151 MHz, MeOD, more than 13 ¹³C signals for compound 10m were observed due to the presence of different rotameric species) δ 173.97, 173.74, 171.02, 170.93, 135.72, 135.67, 128.69, 128.30, 127.69, 127.56, 126.50, 125.94, 72.40, 71.09, 60.75, 60.69, 55.13, 54.21, 53.20, 52.82, 48.25, 48.00, 31.95, 29.97, 25.80, 25.70. HRMS (m/z): [M+H]⁺ calculated for C₁₃H₁₉N₂O₄S⁺ 299.1066, found 299.1052.

Methyl (2S,4S)-1-((2S,3R)-3-amino-2-hydroxy-5-methylhexanoyl)-4-fluoropyrrolidine-2-carboxylate (14a; Method B): White solid; yield 57%; HPLC purity: 95.09%; ¹H NMR (600 MHz, Methanol-d₄) δ 5.46-5.30 (m, 1H), 4.77 (dd, J=7.0, 4.1 Hz, 0.85H), 4.74 (dd, J=7.0, 4.1 Hz, 0.15H), 4.44 (d, J=3.8 Hz, 0.85H), 4.42 (d, J=2.2 Hz, 0.15H), 4.12-4.03 (m, 0.85H), 4.03-3.95 (m, 0.85H), 3.95-3.89 (m, 0.15H), 3.87-3.77 (m, 0.15H), 3.75 (s, 0.45H), 3.74 (s, 2.55H), 3.72-3.67 (m, 0.15H), 3.59-3.53 (m, 0.85H), 2.59-2.41 (m, 2H), 1.84-1.46 (m, 3H), 1.01 (d, J=6.4 Hz, 3H), 0.99 (d, J=6.4 Hz, 3H). ¹³C NMR (151 MHz, MeOD, more than 13 ¹³C signals for compound 14a were observed due to the presence of different rotameric species) δ 174.54, 172.76, 171.78, 171.76, 94.40, 93.22, 92.05, 90.89, 70.62, 69.31, 59.99, 59.13, 55.68, 55.52, 54.96, 54.81, 53.22, 53.06, 52.89, 39.50, 39.32, 38.96, 36.50, 36.36, 30.74, 25.22, 25.16, 23.07, 22.78, 22.58, 22.29. HRMS (m/z): [M+H]⁺ calculated for C₁₃H₂₄N₂O₄F⁺ 291.1720, found 291.1733.

Methyl (S)-5-((2S,3R)-3-amino-2-hydroxy-5-methylhexanoyl)-5-azaspiro[2.4]heptane-6-carboxylate (14b; Method B): White solid; yield 64%; HPLC purity: 97.33%; ¹H NMR (600 MHz, Methanol-d₄) δ 4.94 (d, J=7.5 Hz, 0.15H), 4.65 (dd, J=8.6, 4.5 Hz, 0.85H), 4.44 (d, J=5.1 Hz, 0.85H), 4.38 (d, J=4.7 Hz, 0.15H), 3.81 (dd, J=10.4, 5.1 Hz, 0.85H), 3.76 (s, 0.45H), 3.74 (s, 2.55H), 3.72-3.63 (m, 0.45H), 3.56 (dd, J=10.0, 3.0 Hz, 0.85H), 3.50 (dd, J=8.3, 4.8 Hz, 0.85H), 2.32 (dd, J=12.8, 8.7 Hz, 1H), 1.88 (dd, J=12.9, 4.5 Hz, 0.85H), 1.83-1.69 (m, 1.15H), 1.67-1.48 (m, 3H), 1.00 (d, J=6.5 Hz, 3H), 0.98 (d, J=6.6 Hz, 3H), 0.76-0.52 (m, 4H). ¹³C NMR (151 MHz, MeOD, more than 15 ¹³C signals for compound 14b were observed due to the presence of different rotameric species) δ 175.00, 173.72, 171.52, 171.20, 70.28, 69.10, 61.99, 60.85, 55.87, 55.63, 53.15, 53.11, 52.94, 52.91, 40.32, 39.42, 39.15, 37.96, 30.73, 25.19, 25.14, 23.20, 22.83, 22.60, 22.28, 22.24, 22.16, 15.72, 12.28, 9.34, 6.74. HRMS (m/z): [M+H]⁺ calculated for C₁₅H₂₇N₂O₄⁺ 299.1971, found 299.1968.

Methyl (2S,4S)-1-((2S,3R)-3-amino-2-hydroxy-5-methylhexanoyl)-4-methylpyrrolidine-2-carboxylate (14c; Method B): White solid; yield 67%; HPLC purity: 96.90%; ¹H NMR (600 MHz, Methanol-d₄) δ 4.44 (dd, J=9.4, 7.9 Hz, 1H), 4.38 (d, J=4.5 Hz, 1H), 4.08 (t, J=8.4 Hz, 1H), 3.73 (s, 3H), 3.47 (dt, J=9.9, 5.3 Hz, 1H), 3.15 (t, J=10.0 Hz, 1H), 2.50 (dt, J=13.2, 7.1 Hz, 1H), 2.44-2.35 (m, 1H), 1.77 (dt, J=13.2, 7.2 Hz, 1H), 1.60 (ddd, J=14.2, 8.4, 5.9 Hz, 1H), 1.56-1.48 (m, 2H), 1.12 (d, J=6.5 Hz, 3H), 1.00 (d, J=6.5 Hz, 3H), 0.98 (d, J=6.6 Hz, 3H). ¹³C NMR (151 MHz, MeOD) δ 174.21, 171.40, 69.02, 61.12, 55.20, 53.25, 52.91, 39.26, 37.99, 35.16, 25.18, 23.17, 22.18, 16.80. HRMS (m/z): [M+H]⁺ calculated for C₁₄H₂₇N₂O₄⁺ 287.1971, found 287.1983.

Methyl (2S,4S)-1-((2S,3R)-3-amino-2-hydroxy-5-methylhexanoyl)-4-hydroxypyrrolidine-2-carboxylate (14d; Method B): White solid; yield 47%; HPLC purity: 97.93%; ¹H NMR (600 MHz, Methanol-d₄) δ 4.96 (dd, J=8.6, 1.8 Hz, 0.16H), 4.64 (dd, J=9.3, 3.5 Hz, 0.84H), 4.51-4.45 (m, 0.84H), 4.43 (d, J=4.1 Hz, 0.84H), 4.41-4.38 (m, 0.16H), 4.38 (d, J=2.9 Hz, 0.16H), 3.99 (dd, J=11.0, 5.0 Hz, 1H), 3.76 (s, 0.48H), 3.73 (s, 2.52H), 3.67 (dd, J=7.2, 2.8 Hz, 0.16H), 3.57 (dd, J=10.8, 2.0 Hz, 1H), 3.55-3.52 (m, 0.84H), 2.40 (ddd, J=13.8, 9.4, 4.7 Hz, 1H), 2.17-2.09 (m, 1H), 1.83-1.74 (m, 1H), 1.74-1.62 (m, 1H), 1.60-1.51 (m, 1H), 1.02 (d, J=6.6 Hz, 2.52H), 1.00 (d, J=6.5 Hz, 2.52H), 0.99 (d, J=6.6 Hz, 0.48H), 0.98 (d, J=6.6 Hz, 0.48H). ¹³C NMR (151 MHz, MeOD, more than 13 ¹³C signals for compound 14d were observed due to the presence of different rotameric species) δ 175.18, 173.52, 172.11, 171.91, 70.86, 70.44, 69.07, 68.55, 59.99, 59.19, 56.93, 55.88, 53.10, 52.94, 40.59, 39.60, 39.30, 38.07, 25.24, 25.21, 23.11, 22.85, 22.50, 22.31. HRMS (m/z): [M+H]⁺ calculated for C₁₃H₂₅N₂O₅⁺ 289.1763, found 289.1767.

(2S,3R)-3-amino-2-hydroxy-5-methyl-1-(pyrrolidin-1-yl)hexan-1-one (14e; Method B): White solid; yield 77%; HPLC purity: 99.30%; ¹H NMR (600 MHz, Chloroform-d) δ 4.86 (d, J=3.3 Hz, 1H), 4.25-4.17 (m, 1H), 4.11-4.03 (m, 1H), 4.04-3.97 (m, 1H), 3.99-3.91 (m, 2H), 3.80 (dd, J=3.4, 1.8 Hz, 1H), 2.54-2.45 (m, 2H), 2.44-2.35 (m, 1H), 2.29-2.19 (m, 1H), 2.07-2.00 (m, 2H), 1.47 (d, J=6.6 Hz, 6H). ¹³C NMR (151 MHz, MeOD) δ 171.26, 68.76, 52.87, 47.77, 47.38, 39.62, 26.99, 25.19, 24.97, 22.98, 22.51. HRMS (m/z): [M+H]⁺ calculated for C₁₁H₂₃N₂O₂⁺ 215.1760, found 215.1762.

Methyl ((2S,3R)-3-amino-2-hydroxy-5-methylhexanoyl)-L-leucinate (14f; Method B): White solid; yield 75%; HPLC purity: 99.30%; ¹H NMR (600 MHz, Methanol-d₄) δ 4.49 (dd, J=9.6, 4.8 Hz, 1H), 4.20 (d, J=4.1 Hz, 1H), 3.73 (s, 3H), 3.49 (ddd, J=8.1, 6.2, 4.1 Hz, 1H), 1.81-1.61 (m, 5H), 1.53-1.44 (m, 1H), 0.99 (d, J=6.5 Hz, 3H), 0.98 (d, J=4.9 Hz, 3H), 0.97 (d, J=4.9 Hz, 3H), 0.95 (d, J=6.5 Hz, 3H). ¹³C NMR (151 MHz, MeOD) δ 174.35, 173.60, 71.12, 53.20, 52.84, 52.18, 41.14, 39.22, 26.02, 25.25, 23.19, 22.94, 22.30, 21.95. HRMS (m/z): [M+H]⁺ calculated for C₁₄H₂₉N₂O₄⁺ 289.2127, found 289.2125.

Methyl ((2S,3R)-3-amino-2-hydroxy-5-methylhexanoyl)-L-phenylalaninate (14g; Method B): White solid; yield 72%; HPLC purity: 98.46%; ¹H NMR (600 MHz, Methanol-d₄) δ 7.32-7.21 (m, 5H), 4.73 (dd, J=8.3, 5.7 Hz, 1H), 4.18 (d, J=3.8 Hz, 1H), 3.71 (s, 3H), 3.44 (ddd, J=7.9, 6.2, 3.7 Hz, 1H), 3.20 (dd, J=13.9, 5.8 Hz, 1H), 3.13 (dd, J=13.9, 8.3 Hz, 1H), 1.74-1.65 (m, 1H), 1.54 (ddd, J=14.3, 8.1, 6.3 Hz, 1H), 1.35 (ddd, J=14.3, 7.9, 6.4 Hz, 1H), 0.95 (d, J=6.5 Hz, 3H), 0.92 (d, J=6.5 Hz, 3H). ¹³C NMR (151 MHz, MeOD) δ 173.34, 173.14, 137.91, 130.24, 129.67, 128.12, 70.80, 54.86, 53.16, 52.87, 38.86, 38.01, 25.23, 22.96, 22.36. HRMS (m/z): [M+H]⁺ calculated for C₁₇H₂₇N₂O₄⁺ 323.1971, found 323.1982.

(S)-1-((2S,3R)-3-amino-2-hydroxy-5-methylhexanoyl)pyrrolidine-2-carboxamide (14h; Method C): White solid; yield 45%; HPLC purity: 99.23%; ¹H NMR (500 MHz, Methanol-d₄) δ 4.45 (d, J=2.9 Hz, 1H), 4.43 (dd, J=8.2, 4.6 Hz, 1H), 3.82 (dt, J=10.1, 6.5 Hz, 1H), 3.66 (dt, J=10.1, 6.5 Hz, 1H), 3.54 (dd, J=7.2, 3.2 Hz, 1H), 2.34-2.22 (m, 1H), 2.12-1.95 (m, 3H), 1.83-1.73 (m, 1H), 1.69-1.60 (m, 1H), 1.56-1.47 (m, 1H), 1.01 (d, J=6.8 Hz, 3H), 0.99 (d, J=6.8 Hz, 3H). ¹³C NMR (126 MHz, MeOD) δ 176.96, 171.91, 68.79, 61.51, 52.71, 39.67, 38.88, 30.98, 25.94, 25.21, 22.89, 22.64. HRMS (m/z): [M+H]⁺ calculated for C₁₂H₂₄N₃O₃⁺ 258.1818, found 258.1812.

N—(((S)-1-((2S,3R)-3-amino-2-hydroxy-5-methylhexanoyl)pyrrolidin-2-yl)methyl)methanesulfon-amide (14i; Method C): White solid; yield 62%; HPLC purity: 98.32%; ¹H NMR (600 MHz, Methanol-d₄) δ 4.42 (d, J=2.2 Hz, 1H), 4.20-4.14 (m, 1H), 3.77 (dt, J=10.2, 6.9 Hz, 1H), 3.59-3.51 (m, 3H), 3.14 (dt, J=13.9, 2.8 Hz, 1H), 2.93 (s, 3H), 2.19-2.09 (m, 1H), 2.08-2.00 (m, 1H), 1.99-1.89 (m, 2H), 1.81-1.72 (m, 1H), 1.70-1.62 (m, 1H), 1.59-1.48 (m, 1H), 1.00 (d, J=6.5 Hz, 6H). ¹³C NMR (151 MHz, MeOD) δ 171.97, 68.12, 58.96, 52.72, 48.51, 44.58, 40.06, 39.72, 28.23, 25.22, 25.18, 22.75, 22.71. HRMS (m/z): [M+H]⁺ calculated for C₁₃H₂₈N₃₀₄S⁺ 322.1801, found 322.1798.

(S)-1-((2S,3R)-3-amino-2-hydroxy-5-methylhexanoyl)-N-methoxypyrrolidine-2-carboxamide (14j; Method C): White solid; yield 51%; HPLC purity: 95.63%; ¹H NMR (500 MHz, Methanol-d₄) δ 4.42 (d, J=3.5 Hz, 1H), 4.26 (dd, J=8.0, 5.5 Hz, 1H), 3.87-3.77 (m, 1H), 3.70 (s, 3H), 3.70-3.64 (m, 1H), 3.54-3.48 (m, 1H), 2.28-2.19 (m, 1H), 2.16-2.07 (m, 1H), 2.06-1.94 (m, 2H), 1.87-1.70 (m, 2H), 1.66-1.56 (m, 1H), 1.56-1.46 (m, 1H), 1.00 (d, J=6.6 Hz, 3H), 0.98 (d, J=6.6 Hz, 3H). ¹³C NMR (126 MHz, MeOD) δ 171.90, 171.32, 69.00, 64.34, 59.64, 52.79, 48.57, 39.65, 30.74, 26.01, 25.23, 22.99, 22.50. HRMS (m/z): [M+H]⁺ calculated for C₁₃H₂₆N₃O₄⁺ 288.1923, found 288.1916.

(2S,3R)-1-((S)-2-(1H-tetrazol-5-yl)pyrrolidin-1-yl)-3-amino-2-hydroxy-5-methylhexan-1-one (14k; Method C): White solid; yield 38%; HPLC purity: 96.40%; ¹H NMR (600 MHz, Methanol-d₄) δ 5.43 (dd, J=8.2, 3.2 Hz, 1H), 4.45 (d, J=3.2 Hz, 1H), 3.95-3.84 (m, 2H), 3.51 (dd, J=7.2, 3.2 Hz, 1H), 2.47-2.38 (m, 1H), 2.22-2.10 (m, 3H), 1.77-1.68 (m, 1H), 1.60-1.48 (m, 2H), 0.99 (d, J=6.5 Hz, 3H), 0.95 (d, J=6.5 Hz, 3H). ¹³C NMR (151 MHz, MeOD) δ 171.96, 68.97, 53.33, 52.77, 48.33, 39.54, 32.07, 25.67, 25.17, 22.94, 22.45. HRMS (m/z): [M+H]⁺ calculated for C₁₂H₂₃N₆O₂ 283.1882, found 283.1884.

(S)-1-((2S,3R)-3-amino-2-hydroxy-5-methylhexanoyl)-N-isopentylpyrrolidine-2-carboxamide (14l; Method C): White solid; yield 53%; HPLC purity: 95.08%; ¹H NMR (600 MHz, Methanol-d₄) δ 4.39 (d, J=3.2 Hz, 1H), 4.39-4.37 (m, 1H), 3.80 (dt, J=10.0, 6.8 Hz, 1H), 3.64 (dt, J=10.0, 6.9 Hz, 1H), 3.45-3.41 (m, 1H), 3.26-3.18 (m, 2H), 2.28-2.21 (m, 1H), 2.11-2.03 (m, 1H), 2.01-1.93 (m, 1H), 1.92-1.86 (m, 1H), 1.81-1.75 (m, 1H), 1.66-1.57 (m, 2H), 1.50-1.42 (m, 1H), 1.43-1.37 (m, 2H), 1.00 (d, J=6.6 Hz, 3H), 0.99 (d, J=6.5 Hz, 3H), 0.92 (d, J=6.6 Hz, 6H). ¹³C NMR (151 MHz, MeOD) δ 179.79, 174.19, 61.91, 52.44, 49.57, 48.65, 39.34, 38.76, 30.87, 26.83, 26.08, 25.38, 23.88, 23.05, 22.83, 22.81, 22.71. HRMS (m/z): [M+H]⁺ calculated for C₁₇H₃₄N₃O₃+328.2600, found 328.2596.

Methyl ((2S,3R)-3-amino-2-hydroxy-5-methylhexanoyl)-L-prolyl-L-prolyl-L-alaninate (14m; Method C): White solid; yield 37%; HPLC purity: 97.72%; ¹H NMR (600 MHz, Methanol-d₄) δ 4.73 (dd, J=8.5, 4.7 Hz, 1H), 4.47-4.43 (m, 2H), 4.37 (dd, J=14.7, 7.3 Hz, 1H), 3.85-3.78 (m, 2H), 3.71 (s, 3H), 3.69-3.61 (m, 2H), 3.54 (td, J=7.2, 2.9 Hz, 1H), 2.40-2.30 (m, 1H), 2.28-2.17 (m, 1H), 2.16-1.96 (m, 6H), 1.82-1.72 (m, 1H), 1.72-1.62 (m, 1H), 1.56-1.47 (m, 1H), 1.38 (d, J=7.3 Hz, 3H), 1.00 (d, J=5.4 Hz, 3H), 0.99 (d, J=5.3 Hz, 3H). ¹³C NMR (151 MHz, MeOD) δ 174.54, 174.06, 172.45, 171.54, 68.57, 61.28, 60.19, 52.73, 48.67, 48.48, 39.40, 30.42, 29.23, 25.92, 25.88, 25.20, 22.79, 22.66, 17.25. HRMS (m/z): [M+H]⁺ calculated for C₂₁H₃₇N₄O₆⁺ 441.2713, found 441.2694.

Methyl ((2S,3R)-3-amino-2-hydroxy-5-methylhexanoyl)-L-prolyl-L-valinate (14n; Method C): White powder; yield 57%; HPLC purity: 95.54%; ¹H NMR (600 MHz, Methanol-d₄) δ 4.54 (dd, J=8.3, 5.3 Hz, 1H), 4.41 (d, J=3.4 Hz, 1H), 4.38 (d, J=5.9 Hz, 1H), 3.83-3.76 (m, 1H), 3.71 (s, 3H), 3.68-3.61 (m, 1H), 3.46 (ddd, J=7.6, 6.6, 3.4 Hz, 1H), 2.32-2.23 (m, 1H), 2.11-2.03 (m, 1H), 2.02-1.86 (m, 3H), 1.82-1.72 (m, 1H), 1.60 (ddd, J=14.1, 7.6, 6.7 Hz, 1H), 1.56-1.41 (m, 2H), 1.32-1.23 (m, 1H), 1.00 (d, J=6.6 Hz, 3H), 0.98 (d, J=6.6 Hz, 3H), 0.95 (d, J=6.9 Hz, 3H), 0.92 (d, J=6.9 Hz, 3H). ¹³C NMR (151 MHz, MeOD) δ 174.56, 173.46, 172.03, 69.50, 61.46, 58.39, 52.64, 52.42, 48.62, 40.14, 38.43, 30.60, 26.32, 26.01, 25.31, 23.01, 22.61, 16.05. HRMS (m/z): [M+H]⁺ calculated for C₁₈H₃₄N₃O₅⁺ 372.2498, found 372.2513.

Methyl ((2S,3R)-3-amino-2-hydroxy-5-methylhexanoyl)-L-prolyl-L-phenylalaninate (14o; Method C): White solid; yield 52%; HPLC purity: 99.54%; ¹H NMR (600 MHz, Methanol-d₄) δ 7.32-7.27 (m, 2H), 7.26-7.17 (m, 3H), 4.66 (dd, J=7.7, 6.1 Hz, 1H), 4.47 (dd, J=8.5, 5.0 Hz, 1H), 4.42 (d, J=3.0 Hz, 1H), 3.81-3.74 (m, 1H), 3.67 (s, 3H), 3.67-3.60 (m, 1H), 3.48 (dd, J=7.2, 3.0 Hz, 1H), 3.13 (dd, J=13.9, 6.1 Hz, 1H), 3.05 (dd, J=13.9, 7.7 Hz, 1H), 2.27-2.18 (m, 1H), 2.06-1.87 (m, 3H), 1.82-1.71 (m, 1H), 1.67-1.58 (m, 1H), 1.53-1.45 (m, 1H), 1.00 (d, J=6.6 Hz, 3H), 0.99 (d, J=6.6 Hz, 3H). ¹³C NMR (151 MHz, MeOD) δ 174.28, 172.06, 137.98, 130.36, 129.49, 127.90, 69.13, 61.56, 55.44, 52.65, 52.64, 48.53, 40.05, 38.27, 30.54, 25.90, 25.28, 22.96, 22.66. HRMS (m/z): [M+H]⁺ calculated for C₂₂H₃₄N₃O₅⁺ 420.2498, found 420.2510.

Methyl ((2S,3R)-3-amino-2-hydroxy-5-methylhexanoyl)-L-prolyl-L-leucinate (14p; Method C): White solid; yield 55%; HPLC purity: 98.50%; ¹H NMR (600 MHz, Methanol-d₄) δ 4.49 (dd, J=8.7, 5.0 Hz, 1H), 4.45 (dd, J=9.2, 6.0 Hz, 1H), 4.42 (d, J=3.4 Hz, 1H), 3.83-3.77 (m, 1H), 3.71 (s, 3H), 3.69-3.63 (m, 1H), 3.48 (dd, J=7.2, 3.3 Hz, 1H), 2.32-2.24 (m, 1H), 2.11-2.03 (m, 1H), 2.03-1.94 (m, 2H), 1.82-1.72 (m, 2H), 1.67-1.58 (m, 3H), 1.52-1.45 (m, 1H), 1.00 (d, J=6.6 Hz, 3H), 0.98 (d, J=6.5 Hz, 3H), 0.97 (d, J=6.6 Hz, 3H), 0.92 (d, J=6.5 Hz, 3H). ¹³C NMR (151 MHz, MeOD) δ 174.53, 174.51, 172.02, 69.37, 61.50, 52.67, 52.66, 52.23, 48.61, 41.42, 40.07, 30.59, 25.98, 25.85, 25.30, 23.32, 22.99, 22.62, 21.84. HRMS (m/z): [M+H]⁺ calculated for C₁₉H₃₆N₃O₅⁺ 386.2655, found 386.2640.

Methyl ((2S,3R)-3-amino-2-hydroxy-5-methylhexanoyl)-L-prolyl-L-isoleucinate (14q; Method C): White solid; yield 58%; HPLC purity: 95.15%; ¹H NMR (600 MHz, Methanol-d₄) δ 4.55 (dd, J=8.4, 5.0 Hz, 1H), 4.45 (d, J=3.4 Hz, 1H), 4.33 (d, J=5.8 Hz, 1H), 3.85-3.79 (m, 1H), 3.72 (s, 3H), 3.70-3.64 (m, 1H), 3.53 (dd, J=7.2, 3.4 Hz, 1H), 2.32-2.25 (m, 1H), 2.21-2.12 (m, 1H), 2.11-2.03 (m, 1H), 2.02-1.92 (m, 3H), 1.84-1.70 (m, 2H), 1.68-1.58 (m, 1H), 1.57-1.46 (m, 1H), 1.00 (d, J=6.5 Hz, 3H), 0.99 (d, J=6.5 Hz, 3H), 0.98 (d, J=6.6 Hz, 3H), 0.97 (d, J=6.6 Hz, 3H). ¹³C NMR (151 MHz, MeOD) δ 174.66, 173.43, 171.72, 68.77, 61.49, 59.39, 52.83, 52.49, 48.65, 39.54, 31.85, 30.67, 25.96, 25.22, 22.91, 22.56, 19.55, 18.52. HRMS (m/z): [M+H]⁺ calculated for C₁₉H₃₆N₃O₅⁺ 386.2655, found 386.2648.

(S)-1-((2S,3R)-3-amino-2-hydroxy-5-methylhexanoyl)-N—((S)-1-amino-4-methyl-1-oxopentan-2-yl)pyrrolidine-2-carboxamide (14r; Method C): White solid; yield 45%; HPLC purity: 99.24%; ¹H NMR (600 MHz, Methanol-d₄) δ 4.47 (d, J=2.9 Hz, 1H), 4.45 (dd, J=8.7, 4.8 Hz, 1H), 4.39 (dd, J=10.4, 4.7 Hz, 1H), 3.86-3.79 (m, 1H), 3.73-3.67 (m, 1H), 3.57 (dd, J=7.2, 2.8 Hz, 1H), 2.33-2.24 (m, 1H), 2.11-2.03 (m, 1H), 2.03-1.94 (m, 2H), 1.81-1.70 (m, 2H), 1.71-1.61 (m, 2H), 1.62-1.54 (m, 1H), 1.57-1.49 (m, 1H), 1.01 (d, J=7.0 Hz, 3H), 0.98 (d, J=7.0 Hz, 3H), 0.97 (d, J=6.6 Hz, 3H), 0.93 (d, J=6.5 Hz, 3H). ¹³C NMR (151 MHz, MeOD) δ 177.44, 174.34, 171.95, 68.68, 61.95, 52.97, 52.80, 42.00, 39.47, 30.72, 25.99, 25.90, 25.19, 23.56, 22.92, 22.62, 21.86. HRMS (m/z): [M+H]⁺ calculated for C₁₈H₃₅N₄O₄⁺ 371.2658, found 371.2660.

(S)-1-((2S,3R)-3-amino-2-hydroxy-5-methylhexanoyl)-N—((S)-1-(dimethylamino)-4-methyl-1-oxopentan-2-yl)pyrrolidine-2-carboxamide (14s; Method C): White solid; yield 44%; HPLC purity: 98.75%; ¹H NMR (500 MHz, Methanol-d₄) δ 4.88 (dd, J=10.3, 4.1 Hz, 1H), 4.54-4.41 (m, 1H), 4.26 (d, J=3.6 Hz, 1H), 3.79-3.67 (m, 1H), 3.64-3.54 (m, 1H), 3.15-3.12 (m, 1H), 3.12 (s, 3H), 2.93 (s, 3H), 2.29-2.17 (m, 1H), 2.09-1.98 (m, 1H), 1.98-1.85 (m, 2H), 1.84-1.70 (m, 2H), 1.65-1.54 (m, 1H), 1.51-1.37 (m, 2H), 1.36-1.26 (m, 1H), 0.97 (d, J=6.9 Hz, 3H), 0.95 (d, J=6.9 Hz, 3H), 0.94 (d, J=6.7 Hz, 3H), 0.92 (d, J=6.7 Hz, 3H). ¹³C NMR (126 MHz, MeOD) δ 174.26, 174.02, 173.60, 73.13, 61.54, 51.73, 48.53, 43.17, 41.93, 37.51, 36.16, 30.37, 26.27, 25.74, 25.68, 23.73, 23.52, 22.85, 21.96. HRMS (m/z): [M+H]⁺ calculated for C₂₀H₃₉N₄O₄⁺ 399.2971, found 399.2979.

(S)-1-((2S,3R)-3-amino-2-hydroxy-5-methylhexanoyl)-N—((S)-1-(isopentylamino)-4-methyl-1-oxopentan-2-yl)pyrrolidine-2-carboxamide (14t; Method C): White solid; yield 42%; HPLC purity: 96.48%; ¹H NMR (600 MHz, Methanol-d₄) δ 4.47 (d, J=2.9 Hz, 1H), 4.47-4.43 (m, 1H), 4.35 (dd, J=9.9, 5.4 Hz, 1H), 3.84-3.78 (m, 1H), 3.72-3.66 (m, 1H), 3.57 (dd, J=7.1, 2.8 Hz, 1H), 3.24 (dd, J=12.8, 7.4 Hz, 1H), 3.16 (dd, J=13.3, 7.2 Hz, 1H), 2.33-2.22 (m, 1H), 2.10-1.90 (m, 3H), 1.82-1.57 (m, 5H), 1.57-1.48 (m, 2H), 1.47-1.36 (m, 2H), 1.01 (d, J=6.9 Hz, 3H), 0.99 (d, J=6.9 Hz, 3H), 0.96 (d, J=6.7 Hz, 3H), 0.94 (d, J=6.9 Hz, 3H), 0.92 (d, J=6.7 Hz, 3H), 0.91 (d, J=6.7 Hz, 3H). ¹³C NMR (151 MHz, MeOD) δ 174.43, 174.23, 171.86, 68.69, 61.85, 53.39, 52.79, 48.68, 42.17, 39.49, 39.30, 38.68, 30.75, 26.83, 25.96, 25.90, 25.20, 23.43, 22.89, 22.82, 22.79, 22.64, 22.10. HRMS (m/z): [M+H]⁺ calculated for C₂₃H₄₅N₄O₄⁺ 441.3441, found 441.3448.

(S)-1-((2S,3R)-3-amino-2-hydroxy-5-methylhexanoyl)-N—((S)-1-(methoxyamino)-4-methyl-1-oxopentan-2-yl)pyrrolidine-2-carboxamide (14u; Method C): White solid; yield 32%; HPLC purity: 97.35%; ¹H NMR (600 MHz, Methanol-d₄) δ 4.46 (dd, J=8.2, 6.2 Hz, 1H), 4.28 (d, J=3.4 Hz, 1H), 4.27-4.24 (m, 1H), 3.78-3.73 (m, 1H), 3.59 (dd, J=9.9, 7.2 Hz, 1H), 3.15 (ddd, J=7.6, 6.2, 3.5 Hz, 1H), 2.30-2.22 (m, 1H), 2.10-2.02 (m, 1H), 1.98-1.85 (m, 2H), 1.83-1.69 (m, 2H), 1.68-1.59 (m, 1H), 1.57-1.48 (m, 1H), 1.49-1.41 (m, 1H), 1.36-1.24 (m, 4H), 0.97 (d, J=6.6 Hz, 3H), 0.96 (d, J=6.6 Hz, 3H), 0.94 (d, J=6.5 Hz, 3H), 0.92 (d, J=6.5 Hz, 3H). ¹³C NMR (151 MHz, MeOD) δ 174.22, 173.71, 171.18, 72.93, 64.20, 61.68, 51.77, 50.85, 49.57, 43.10, 41.99, 30.41, 26.29, 25.72, 23.47, 23.28, 22.86, 22.20. HRMS (m/z): [M+H]⁺ calculated for C₁₉H₃₇N₄O₅⁺ 401.2764, found 401.2764.

(S)-1-((2S,3R)-3-amino-2-hydroxy-5-methylhexanoyl)-N—((S)-1-(azetidin-1-yl)-4-methyl-1-oxopentan-2-yl)pyrrolidine-2-carboxamide (14v; Method C): White solid; yield 27%; HPLC purity: 96.41%; ¹H NMR (600 MHz, Methanol-d₄) δ 4.50-4.48 (m, 1H), 4.48-4.46 (m, 1H), 4.46-4.40 (m, 1H), 4.32-4.25 (m, 1H), 4.29 (d, J=3.6 Hz, 1H), 4.05-3.94 (m, 2H), 3.79-3.72 (m, 1H), 3.64-3.57 (m, 1H), 3.18-3.14 (m, 1H), 2.35-2.22 (m, 2H), 2.09-2.00 (m, 1H), 1.99-1.85 (m, 1H), 1.84-1.73 (m, 1H), 1.67-1.52 (m, 2H), 1.51-1.43 (m, 1H), 1.38-1.31 (m, 1H), 0.98 (d, J=6.6 Hz, 3H), 0.95 (d, J=6.6 Hz, 3H), 0.94 (d, J=6.5 Hz, 3H), 0.92 (d, J=6.5 Hz, 3H). ¹³C NMR (151 MHz, MeOD) δ 173.79, 173.71, 173.27, 61.60, 52.16, 51.80, 49.57, 48.43, 41.51, 30.52, 26.25, 25.83, 25.73, 23.50, 23.48, 22.85, 22.11, 16.27. HRMS (m/z): [M+H]⁺ calculated for C₂₁H₃₉N₄O₄⁺ 411.2971, found 411.2981.

(S)-1-((2S,3R)-3-amino-2-hydroxy-5-methylhexanoyl)-N—((S)-1-((N,N-dimethylsulfamoyl)-amino)-4-methyl-1-oxopentan-2-yl)pyrrolidine-2-carboxamide (14w; Method C): White solid; yield 29%; HPLC purity: 98.14%; ¹H NMR (600 MHz, Methanol-d₄) δ 4.61-4.58 (m, 0.21H), 4.48 (d, J=2.6 Hz, 0.79H), 4.44 (dd, J=8.5, 4.8 Hz, 0.79H), 4.38 (d, J=9.3 Hz, 0.21H), 4.35 (dd, J=9.5, 5.4 Hz, 0.79H), 4.26 (d, J=4.0 Hz, 0.21H), 3.79 (dd, J=10.3, 6.3 Hz, 0.79H), 3.69 (dd, J=10.0, 5.9 Hz, 1H), 3.62 (dd, J=7.2, 2.5 Hz, 0.79H), 3.57 (ddd, J=12.0, 10.1, 7.1 Hz, 0.21H), 3.53-3.46 (m, 0.21H), 2.79 (s, 4.74H), 2.76 (s, 1.26H), 2.33-2.24 (m, 1H), 2.23-2.16 (m, 0.21H), 2.10-1.94 (m, 2.79H), 1.84-1.70 (m, 2H), 1.70-1.63 (m, 1H), 1.63-1.48 (m, 3H), 1.02 (d, J=4.7 Hz, 2.37H), 1.01 (d, J=4.4 Hz, 2.37H), 0.98 (d, J=4.7 Hz, 0.63H), 0.96 (d, J=4.4 Hz, 0.63H), 0.96 (d, J=6.6 Hz, 3H), 0.94 (d, J=6.6 Hz, 3H). ¹³C NMR (151 MHz, MeOD, more than 20 ¹³C signals for compound 14w were observed due to the presence of different rotameric species) δ 178.25, 177.89, 174.50, 173.78, 172.62, 172.21, 70.32, 68.44, 63.26, 62.22, 55.44, 54.80, 52.86, 52.67, 49.57, 48.79, 43.07, 42.72, 39.76, 39.53, 39.10, 39.00, 33.04, 30.63, 26.45, 26.08, 26.00, 25.34, 25.23, 23.68, 23.66, 22.96, 22.88, 22.84, 22.78, 22.45, 22.12, 21.80. HRMS (m/z): [M+H]⁺ calculated for C₂₀H₄₀N₅O₆S⁺ 478.2699, found 478.2688.

(S)-1-((2S,3R)-3-amino-2-hydroxy-5-methylhexanoyl)-N—((S)-1-((2-fluoroethyl)amino)-4-methyl-1-oxopentan-2-yl)pyrrolidine-2-carboxamide (14x; Method C): White solid; yield 47%; HPLC purity: 96.66%; ¹H NMR (600 MHz, Methanol-d₄) δ 4.51-4.47 (m, 1H), 4.45 (d, J=2.9 Hz, 1H), 4.44-4.38 (m, 3H), 3.81 (dt, J=10.2, 6.7 Hz, 1H), 3.68 (dt, J=10.1, 7.0 Hz, 1H), 3.60-3.44 (m, 3H), 2.28 (dt, J=13.4, 7.2 Hz, 1H), 2.10-1.90 (m, 2H), 1.82-1.46 (m, 7H), 1.01 (d, J=6.6 Hz, 3H), 1.00 (d, J=6.6 Hz, 3H), 0.97 (d, J=6.7 Hz, 3H), 0.93 (d, J=6.7 Hz, 3H). ¹³C NMR (126 MHz, MeOD) δ 175.07, 174.29, 174.13, 83.73, 82.40, 73.08, 61.96, 53.21, 51.71, 43.57, 41.91, 41.16, 40.99, 30.42, 26.34, 25.85, 25.75, 23.48, 22.92, 21.96. HRMS (m/z): [M+H]⁺ calculated for C₂₀H₃₈N₄O₄F⁺ 417.2877, found 417.2889.

(S)-1-((2S,3R)-3-amino-2-hydroxy-5-methylhexanoyl)-N—((S)-1-(tert-butylamino)-4-methyl-1-oxopentan-2-yl)pyrrolidine-2-carboxamide (14y; Method C): White solid; yield 51%; HPLC purity: 96.71%; ¹H NMR (600 MHz, Methanol-d₄) δ 4.45 (d, J=2.4 Hz, 1H), 4.44-4.42 (m, 0.75H), 4.41 (dd, J=8.3, 5.7 Hz, 0.25H), 4.37-4.27 (m, 1H), 3.86-3.76 (m, 1H), 3.68 (dt, J=10.0, 6.7 Hz, 1H), 3.61-3.52 (m, 1H), 2.31-2.20 (m, 1H), 2.13-1.90 (m, 3H), 1.79-1.61 (m, 3H), 1.61-1.47 (m, 3H), 1.34 (s, 2.25H), 1.32 (s, 6.75H), 1.01 (d, J=6.8 Hz, 3H), 0.99 (d, J=6.8 Hz, 3H), 0.96 (d, J=6.6 Hz, 3H), 0.92 (d, J=6.6 Hz, 2.25H), 0.91 (d, J=6.6 Hz, 0.75H). ¹³C NMR (126 MHz, MeOD, more than 22 ¹³C signals for compound 14y were observed due to the presence of different rotameric species) δ 174.08, 174.05, 173.91, 173.81, 173.75, 173.52, 73.25, 73.11, 62.06, 61.85, 53.64, 52.24, 52.07, 51.69, 43.48, 43.22, 42.01, 41.57, 41.57, 38.89, 30.49, 30.28, 28.93, 28.86, 26.49, 26.32, 26.12, 25.86, 25.75, 25.72, 23.50, 22.91, 22.86, 22.13, 21.65. HRMS (m/z): [M+H]⁺ calculated for C₂₂H₄₃N₄O₄⁺ 427.3284, found 427.3298.

(S)-1-((2S,3R)-3-amino-2-hydroxy-5-methylhexanoyl)-N—((S)-4-methyl-1-oxo-1-(phenethylamino)pentan-2-yl)pyrrolidine-2-carboxamide (14z; Method C): White solid; yield 53%; HPLC purity: 97.80%; ¹H NMR (600 MHz, Methanol-d₄) δ 7.30-7.25 (m, 2H), 7.24-7.16 (m, 3H), 4.44 (d, J=3.2 Hz, 1H), 4.43 (dd, J=5.6, 2.6 Hz, 1H), 3.83-3.77 (m, 1H), 3.67 (dt, J=9.8, 6.6 Hz, 1H), 3.55 (dd, J=7.2, 3.0 Hz, 1H), 3.53-3.46 (m, 1H), 3.39-3.34 (m, 1H), 2.83-2.76 (m, 2H), 2.28-2.20 (m, 1H), 2.08-2.01 (m, 1H), 2.02-1.94 (m, 1H), 1.95-1.88 (m, 1H), 1.81-1.72 (m, 1H), 1.69-1.60 (m, 2H), 1.58-1.42 (m, 4H), 1.01 (d, J=6.6 Hz, 3H), 1.00 (d, J=6.6 Hz, 3H), 0.93 (d, J=6.7 Hz, 3H), 0.89 (d, J=6.7 Hz, 3H). ¹³C NMR (126 MHz, MeOD) δ 174.66, 174.14, 174.02, 140.39, 129.86, 129.47, 127.35, 73.07, 61.86, 53.28, 51.73, 43.48, 42.03, 41.82, 38.88, 36.29, 30.40, 26.33, 25.78, 25.75, 23.50, 23.45, 22.90, 22.01. HRMS (m/z): [M+H]⁺ calculated for C₂₆H₄₃N₄O₄⁺ 475.3284, found 475.3297.

(S)-1-((2S,3R)-3-amino-2-hydroxy-5-methylhexanoyl)-N—((S)-4-methyl-1-((2-(naphthalen-2-yl)ethyl)amino)-1-oxopentan-2-yl)pyrrolidine-2-carboxamide (14aa; Method C): White solid; yield 56%; HPLC purity: 95.99%; ¹H NMR (600 MHz, Methanol-d₄) δ 7.82-7.78 (m, 3H), 7.66 (br.s, 1H), 7.47-7.37 (m, 3H), 4.42 (d, J=3.1 Hz, 1H), 4.38 (dd, J=8.4, 5.4 Hz, 1H), 4.30 (dd, J=9.9, 5.4 Hz, 1H), 3.79-3.72 (m, 1H), 3.70-3.62 (m, 1H), 3.61-3.56 (m, 1H), 3.56-3.51 (m, 1H), 3.50-3.43 (m, 1H), 3.04-2.93 (m, 3H), 2.19-2.07 (m, 1H), 2.00-1.85 (m, 2H), 1.82-1.71 (m, 2H), 1.72-1.60 (m, 1H), 1.60-1.42 (m, 2H), 1.42-1.33 (m, 1H), 1.01 (d, J=6.5 Hz, 3H), 0.99 (d, J=6.5 Hz, 3H), 0.98-0.93 (m, 1H), 0.93-0.85 (m, 1H), 0.84 (d, J=6.6 Hz, 3H), 0.81 (d, J=6.6 Hz, 3H). ¹³C NMR (126 MHz, MeOD) δ 174.76, 174.07, 174.02, 137.87, 135.02, 133.75, 129.10, 128.62, 128.59, 128.34, 128.31, 126.98, 126.41, 72.98, 61.81, 53.30, 51.65, 43.46, 41.99, 41.40, 36.24, 30.30, 26.29, 25.71, 23.47, 23.38, 22.90, 21.92. HRMS (m/z): [M+H]⁺ calculated for C₃₀H₄₅N₄O₄⁺ 525.3441, found 525.3459.

(S)—N—((S)-1-((2-(1H-indol-6-yl)ethyl)amino)-4-methyl-1-oxopentan-2-yl)-1-((2S,3R)-3-amino-2-hydroxy-5-methylhexanoyl)pyrrolidine-2-carboxamide (14ab; Method C): White solid; yield 53%; HPLC purity: 99.57%; ¹H NMR (600 MHz, Methanol-d₄) δ 7.55 (dt, J=8.0, 1.0 Hz, 1H), 7.32 (dt, J=8.1, 0.9 Hz, 1H), 7.10-7.06 (m, 2H), 7.00 (ddd, J=8.0, 7.0, 1.0 Hz, 1H), 4.44 (d, J=3.0 Hz, 1H), 4.41 (dd, J=8.5, 5.3 Hz, 1H), 4.33 (dd, J=9.9, 5.4 Hz, 1H), 3.78 (dt, J=10.0, 6.8 Hz, 1H), 3.63 (dt, J=9.9, 6.7 Hz, 1H), 3.61-3.53 (m, 2H), 3.47-3.42 (m, 1H), 2.98-2.92 (m, 2H), 2.22-2.15 (m, 1H), 2.04-1.90 (m, 1H), 1.88-1.79 (m, 1H), 1.80-1.72 (m, 1H), 1.69-1.60 (m, 2H), 1.59-1.43 (m, 3H), 1.00 (d, J=6.5 Hz, 3H), 0.99 (d, J=6.5 Hz, 3H), 0.98-0.92 (m, 1H), 0.91 (d, J=6.6 Hz, 3H), 0.88 (d, J=6.6 Hz, 3H). ¹³C NMR (126 MHz, MeOD) δ 174.65, 174.16, 174.04, 138.11, 131.33, 128.80, 123.53, 122.31, 119.61, 119.31, 113.02, 112.25, 73.02, 61.85, 53.33, 51.66, 43.47, 41.90, 41.16, 30.34, 26.32, 25.97, 25.78, 25.73, 23.47, 23.45, 22.89, 21.95. HRMS (m/z): [M+H]⁺ calculated for C₂₈H₄₄N₅O₄⁺ 514.3393, found 514.3383.

(S)—N—((S)-1-((2-([1,1′-biphenyl]-4-yl)ethyl)amino)-4-methyl-1-oxopentan-2-yl)-1-((2S,3R)-3-amino-2-hydroxy-5-methylhexanoyl)pyrrolidine-2-carboxamide (14ac; Method C): White solid; yield 55%; HPLC purity: 99.48%; ¹H NMR (600 MHz, Methanol-d₄) δ 7.60-7.56 (m, 2H), 7.56-7.52 (m, 2H), 7.43-7.38 (m, 2H), 7.33-7.27 (m, 3H), 4.43 (d, J=3.0 Hz, 1H), 4.43-4.39 (m, 1H), 4.33 (dd, J=9.9, 5.4 Hz, 1H), 3.81-3.74 (m, 1H), 3.67-3.60 (m, 1H), 3.58-3.50 (m, 2H), 3.45-3.38 (m, 1H), 2.89-2.82 (m, 2H), 2.24-2.17 (m, 1H), 2.04-1.96 (m, 1H), 1.96-1.84 (m, 2H), 1.80-1.71 (m, 1H), 1.70-1.59 (m, 2H), 1.58-1.41 (m, 3H), 1.01 (d, J=6.8 Hz, 3H), 0.99 (d, J=6.8 Hz, 3H), 0.91 (d, J=6.6 Hz, 3H), 0.88 (d, J=6.6 Hz, 3H). ¹³C NMR (126 MHz, MeOD) δ 174.74, 174.13, 174.09, 142.16, 140.49, 139.55, 130.42, 129.82, 128.18, 128.01, 127.79, 73.02, 61.88, 53.33, 51.72, 48.62, 43.53, 42.01, 41.64, 38.88, 35.82, 30.40, 26.33, 25.80, 25.74, 23.46, 22.91, 22.01. HRMS (m/z): [M+H]⁺ calculated for C₃₂H₄₇N₄O₄⁺ 551.3597, found 551.3613.

tert-butyl ((2S,3R)-3-amino-2-hydroxy-5-methylhexanoyl)-L-prolinate (15a; Method C): White powder; yield 77%; HPLC purity: 99.47%; ¹H NMR (600 MHz, Methanol-d₄) δ 4.61 (dd, J=8.6, 1.7 Hz, 0.14H), 4.40 (d, J=4.4 Hz, 0.86H), 4.37 (dd, J=8.7, 4.7 Hz, 1H), 3.82 (dt, J=10.4, 7.0 Hz, 0.86H), 3.71-3.65 (m, 1H), 3.65-3.55 (m, 0.28H), 3.53-3.46 (m, 0.86H), 2.32-2.23 (m, 1H), 2.16-2.11 (m, 0.14H), 2.09-1.86 (m, 2.86H), 1.81-1.69 (m, 1H), 1.67-1.56 (m, 1H), 1.56-1.49 (m, 1H), 1.49 (s, 1.26H), 1.47 (s, 7.74H), 1.00 (d, J=6.6 Hz, 3H), 0.98 (d, J=6.6 Hz, 3H). ¹³C NMR (151 MHz, MeOD, more than 16 ¹³C signals for compound 15a were observed due to the presence of different rotameric species) δ 173.87, 173.02, 171.60, 171.24, 83.18, 83.06, 70.55, 69.29, 62.02, 61.43, 53.02, 52.55, 48.44, 48.41, 39.60, 39.43, 32.23, 29.98, 28.20, 28.15, 25.78, 25.25, 23.15, 22.82, 22.73, 22.62, 22.36, 22.26. HRMS (m/z): [M+H]⁺ calculated for C₁₆H₃₁N₂O₄⁺ 315.2284, found 315.2288.

Cyclopentyl ((2S,3R)-3-amino-2-hydroxy-5-methylhexanoyl)-L-prolinate (15b; Method C): White solid; yield 67%; HPLC purity: 98.68%; ¹H NMR (600 MHz, Methanol-d₄) δ 5.21-5.16 (m, 1H), 4.72-4.67 (m, 0.13H), 4.43 (dd, J=9.0, 4.4 Hz, 0.87H), 4.41 (d, J=3.0 Hz, 0.87H), 4.38 (d, J=3.0 Hz, 0.13H), 3.86-3.79 (m, 0.87H), 3.70 (dt, J=10.2, 6.4 Hz, 1H), 3.66-3.55 (m, 0.26H), 3.54-3.47 (m, 0.87H), 2.34-2.23 (m, 1H), 2.15-2.09 (m, 0.13H), 2.09-2.00 (m, 1.87H), 1.99-1.92 (m, 1H), 1.92-1.83 (m, 2H), 1.82-1.59 (m, 8H), 1.58-1.47 (m, 1H), 1.00 (d, J=6.4 Hz, 3H), 0.98 (d, J=6.4 Hz, 3H). ¹³C NMR (151 MHz, MeOD, more than 17 ¹³C signals for compound 15b were observed due to the presence of different rotameric species) δ 174.43, 173.55, 171.57, 171.21, 79.89, 79.78, 70.43, 69.02, 61.59, 60.94, 53.04, 48.46, 48.43, 39.47, 39.30, 33.57, 33.54, 33.47, 33.42, 32.25, 29.95, 25.86, 25.22, 24.68, 24.65, 23.06, 22.78, 22.64, 22.41, 22.30. HRMS (m/z): [M+H]⁺ calculated for C₁₇H₃₁N₂O₄⁺ 327.2284, found 327.2282.

### Exemplary Biological Experiments and Results

Antibodies. Antibodies used include: GSDMD rabbit polyclonal Ab (Novus Biologicals, NBP233422), CARD8 C-terminus rabbit polyclonal Ab (Abcam, Ab24186), PARP rabbit polyclonal Ab (Cell Signaling Tech, 9542), GAPDH rabbit monoclonal Ab (Cell Signaling Tech, 14C10), mouse GSDMD rabbit monoclonal Ab [EPR19828](Abcam, ab209845), DPP9 rabbit polyclonal (Abcam, ab42080), PEPD rabbit monoclonal Ab (EPR16959; Abcam, ab197890), XPNPEP1 (Abcam, ab123929), GSDMD rabbit monoclonal Ab [EPR20829-408](Abcam, ab215203), MYC tag rabbit monoclonal Ab (Cell Signaling Tech, 2278), HA tag rabbit monoclonal Ab (Cell Signaling Tech, 3724), IRDye 800CW donkey anti-rabbit (925-32211), IRDye 680RD donkey anti-rabbit (925-68073), IRDye 800CW donkey anti-mouse (925-32212), IRDye 680RD donkey anti-mouse (925-68072), IRDye 800CW donkey anti-goat (925-32214).

Cell Culture. HEK293T, THP-1, RAW264.7 cells were purchased from ATCC. MV4;11, OCI-AML2 cells were purchased from DSMZ. N/TERT1 cells were a gift from the Rheinwald Lab (Dickson et al., 2000). HEK293T and RAW264.7 cells were grown in Dulbecco's Modified Eagle's Medium (DMEM) with L-glutamine and 10% fetal bovine serum (FBS). N/TERT1 cells were grown in Keratinocyte serum free medium (KSFM) supplemented with 1× penicillin/streptomycin, bovine pituitary extract (25 μg/ml) and epidermal growth factor (EGF) (0.2 ng/ml). All other cell lines were grown in Roswell Park Memorial Institute (RPMI) medium 1640 with L-glutamine and 10% fetal bovine serum (FBS). All cells were grown at 37° C. in a 5% CO₂ atmosphere incubator. Cell lines were regularly tested for mycoplasma using the MycoAlert™ Mycoplasma Detection Kit (Lonza).

Mouse BMDM isolation and culture. Bone marrow was harvested from the femurs and tibias of 7-12-week-old C57BL/6J mice. Briefly, femurs and tibias were harvested from mice and crushed with a mortar and pestle in cold 1×PBS supplemented with 2.5% FBS. The mixture was passed through a 70-μm nylon cell strainer. RBCs were lysed for 4-5 min on ice in 1×RBC lysis buffer (Biolegend) and cells were centrifuged at 300×g for 5 min at 4° C. The cell pellet was washed in cold 1×PBS supplemented with 2.5% FBS before being passed again through in a 70-μm nylon cell strainer and counted. Counted cells were plated on non-tissue culture 10-cm plates at 5-10×10⁶ cells per plate in DMEM supplemented with 10% FBS and 15-20% L-cell media. Cells were incubated at 37° C. for 6 days before replating and assaying as indicated.

Human primary cell isolation and culture. Isolated human primary cells were obtained from Astarte Biologics. All cells were >90% purity, as validated by flow cytometry by Astarte Biologics. T cells were thawed in RPMI-1640 medium supplemented with 10% FBS and cultured in RPMI-1640 medium, 10% fetal bovine serum (FBS), and 30 U/mL IL-2 (Peprotech).

Cloning. CASP1^{-/-} MV4;11 cells¹²; CARD8^{-/-} MV4;11, THP-1, OCI-AML2 cells¹⁴; DPP8/9^{-/-} THP-1¹²; PEPD^{-/-}, XPNPEP1^{-/-}, PEPD XPNPEP1^{-/-} THP-1 cells were generated. Plasmids for CARD8 variants, CASP1, GSDMD, dTAG-CARD8^{ZUC} were cloned as described previously (Hollingsworth et al., 2021; Johnson et al., 2018; Sharif et al., 2021).

Protein purification. Human prolidase (PEPD) was generated and purified. XPNPEP1 in pET15-b containing a 6× His tag was expressed in E. coli Rosetta DE3 cells. Cells were induced with IPTG for 16 h at 37° C., pelleted and lysed in buffer A (20 mM Tris-HCl (pH 7.9), 500 mM NaCl, and 10% (v/v) glycerol) by sonication. Affinity purification chromatography was performed using TALON resin and according to previously published protocols (Li et al., 2008).

Substrate assays. For the PEPD assay, a solution of substrate (Ala-Pro) was prepared in DMSO. 24 μL of 50 nM recombinant human PEPD were added to a 384-well, black, clear-bottom plate (Corning) with 1 μL dipeptide Ala-Pro (final conc. 40 μM). Alanine liberated was measured as increasing fluorescence signal (Resorufin, Ex/Em: 535/587 nm) recorded at 25° C. using an L-alanine assay kit (Abcam, ab83394) at 25° C. according to manufacturer's instructions. For the AMC reporter assays, experiments were performed with cell lysates. 5 μL solution of substrate (2.5 mM Ala-AMC) was added the mixture of 10 μL HEK293T cell lysates (2.5 mg/ml) and 10 μL of indicated compounds in a 384-well, black, clear-bottom plate (Corning) to initiate the reaction. Substrate cleavage was measured as increasing fluorescence signal (Ex/Em: 380/460 nm) recorded at 25° C. for 20 mins. For XPNPEP1 enzymes (XPNPEP1 3.5 nM) were plated on a black 384-well clear bottom plate and treated with the indicated doses of compounds. H-Lys(abz)-Pro-Pro-pNA substrate was added to a final concentration of 100 μM, and fluorescence was monitored (Ex/Em: 320/410) for 60 mins.

CellTiter-Glo cell viability and CytoTox-Fluor cell death assays. Cells were plated (2,000 cells per well) in white, 384-well clear-bottom plates (Corning) using an EL406 Microplate Washer/Dispenser (BioTek) in 25 μL final volume of medium. To the cell plates were added compounds at different concentrations using a pintool (CyBio) and the plates were allowed to incubate in the incubator. After incubation for indicated times, CytoTox-Fluor reagent (Promega, G9262) was added according to the manufacturer's protocol. The assay plates were then incubated for another 30 min before fluorescence was recorded using a Cytation 5 Cell Imaging Multi-Mode Reader (BioTek). Next, CellTiter-Glo reagent (Promega, G7573) was subsequently added to the assay plates following the manufacturer's protocol. Assay plates were shaken on an orbital shaker for 2 min and incubated at 25° C. for 10 min. Luminescence was then read using a Cytation 5 Cell Imaging Multi-Mode Reader (BioTek).

Propidium iodideflux analysis. 2×10⁴ MV4;11 or OCI-AML2 cells were plated in 384-well, black, clear-bottom plate (Corning) in RPMI medium. Cells were then treated as indicated and propidium iodide (PI) was added at a final concentration of 10 μM. PI fluorescence was measured at (Ex/Em: 535/617 nm) recorded at 37° C. every 5 mins in a Cytation 5 Cell Imaging Multi-Mode Reader (BioTek). The obtained measurements were baseline corrected to vehicle/DMSO and normalized to maximum response.

LDH cytotoxicity and immunoblotting assays. HEK293T cells were transiently transfected and treated with inhibitors as indicated. MV4;11, OCI AML2, THP-1, and RAW264.7 cells were plated in 12-well culture plates at 5×10⁵ cells/well and treated as indicated. Supernatants were analyzed for LDH activity using the Pierce LDH Cytotoxicity Assay Kit (Life Technologies). LDH activity was quantified relative to a lysis control where cells were lysed in 80 μL of a 9% Triton X-100 solution. For immunoblotting, cells were washed 2× in PBS (pH=7.4), resuspended in PBS, and lysed by sonication. Protein concentrations were determined and normalized using the DCA Protein Assay kit (Bio-Rad). The samples were separated by SDS-PAGE, immunoblotted, and visualized using the Odyssey Imaging System (Li-Cor).

Transient transfections. HEK293T cells were plated in 6-well culture plates at 5×10⁵ cells/well in DMEM. The next day, the indicated plasmids were mixed with an empty vector to a total of 2.0 μg DNA in 125 μL Opti-MEM and transfected using FuGENE HD (Promega) according to the manufacturer's protocol.

dTAG-CARD8 Assay. HEK293T cells stably expressing CASP1 and GSDMD were seeded at 1.5×10⁵ cells per well in 12-well tissue culture dishes. After 48 h, the cells were transfected with plasmids encoding dTAG-CARD8-ZUC (0.5 μg), CARD8 FIIND-S297A (0.3 μg) and RFP (0.2 μg) with FuGENE HD, according to the manufacturer's instructions (Promega). After 24 h, cells were treated with DMSO, dTAG13 (500 nM) and indicated compounds for 6 h prior to LDH release and immunoblot analyses.

Statistical analysis. Two-sided Student's t tests were used for significance testing unless stated otherwise. P values less than 0.05 were considered to be significant. Graphs and error bars represent means±SEM of three independent experiments, unless stated otherwise. The investigators were not blinded in all experiments.

IC50 values for additional compounds are given in Table 5. Formula IA, where R¹ is isobutanyl (“CQ31”), inhibits PEPD and XPNPEP1 with half-maximal inhibitory concentration (IC50) values of 0.67 μM and 122 μM, respectively. Formula IA, where R¹ is isobutanyl, has selectivity for PEPD over XPNPEP1. Without being bound by any theory, the selectivity may be due to substrate preferences of these enzymes. Although this Formula IA compound only weakly inhibited XPNPEP1, XPNPEP1 blockade contributed to compound-induced CARD8 activation, as knockout of both PEPD and XPNPEP1 generated compound-resistant cells.

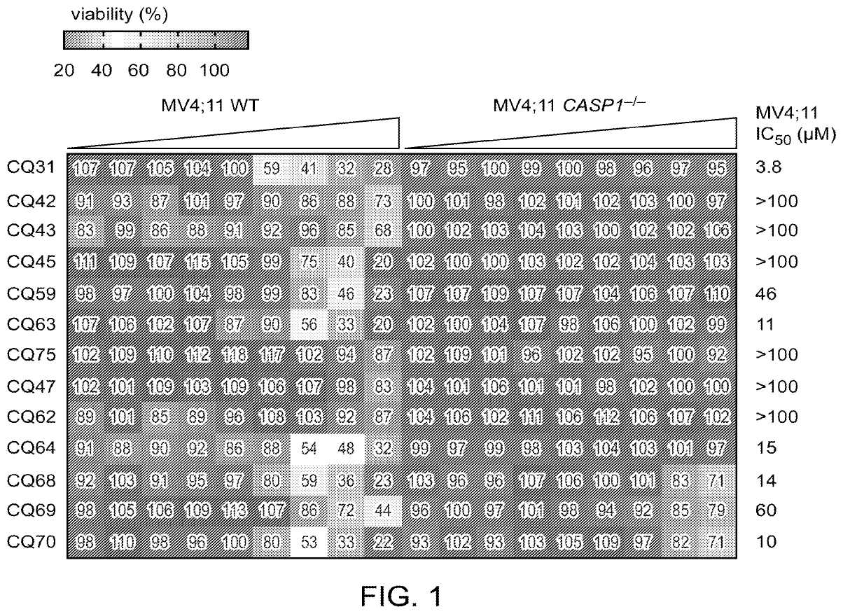

These compounds were tested for their ability to inhibit the enzymatic activities of purified PEPD and XPNPEP1 as well as to induce cell death in wild-type (WT) MV4;11 cells, which express a functional CARD8 inflammasome. Notably, the compounds in CASP1 knockout MV4;11 cells were tested to confirm that cell death was due to inflammasome activation. Most of these compounds had similar or slightly reduced PEPD and XPNEP1 inhibitory activity (Table 1), and all had reduced cytotoxicity (FIG. 1). Notably, three compounds (CQ42, CQ47, and CQ62) were markedly less potent inhibitors of PEPD (IC50s>4 μM) and almost completely inactive against MV4; 1 cells. One of these inactive compounds, CQ47, was the most active of this series against XPNPEP1 (IC50=33 μM), further highlighting the potent PEPD blockade. CQ75 retained PEPD inhibition but lost cytotoxicity, which, without being bound by any theory, may be due to the hydrophilic hydroxyl group compromising cell permeability (Table 1, FIG. 1). Regardless, these data indicated that PEPD and XPNPEP1 broadly accommodate many N-terminal side chains, and that modification on this part of the molecule was unlikely to substantially improve bioactivity.

TABLE 5
Inhibition of PEPD and XPNPEP1 by N-terminal analogues

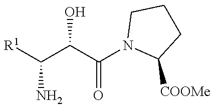

| Compound | R¹ | PEPD IC₅₀ (μM) | XPNPEP1 IC₅₀ (μM) |
|---|---|---|---|
| CQ31 | 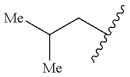 [SMILES: *CC(C)C] | 0.67 | 122 |
| CQ42 | 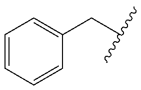 [SMILES: *Cc1ccccc1] | 7.6 | 241 |
| CQ43 | 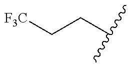 [SMILES: *CCC(F)(F)F] | 1.3 | 115 |
| CQ45 | 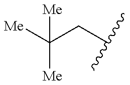 [SMILES: *CC(C)(C)C] | 1.5 | 57 |
| CQ59 | 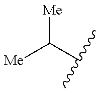 [SMILES: *C(C)C] | 1.8 | 190 |
| CQ63 | 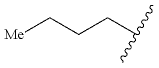 [SMILES: *CCCC] | 0.53 | 92 |
| CQ75 | 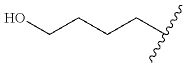 [SMILES: *CCCCO] | 0.30 | 115 |
| CQ47 | 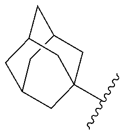 [SMILES: *C12CC3CC(CC(C3)C1)C2] | 12 | 33 |
| CQ62 | 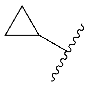 [SMILES: *C1CC1] | 4.2 | 66 |
| CQ64 | 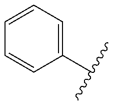 [SMILES: *c1ccccc1] | 2.8 | 203 |
| CQ68 | 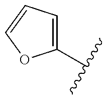 [SMILES: *c1ccco1] | 1.7 | 69 |
| CQ69 | 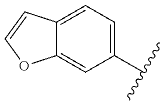 [SMILES: *c1ccc2ccoc2c1] | 1.8 | 47 |
| CQ70 | 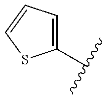 [SMILES: *c1cccs1] | 2.0 | 65 |

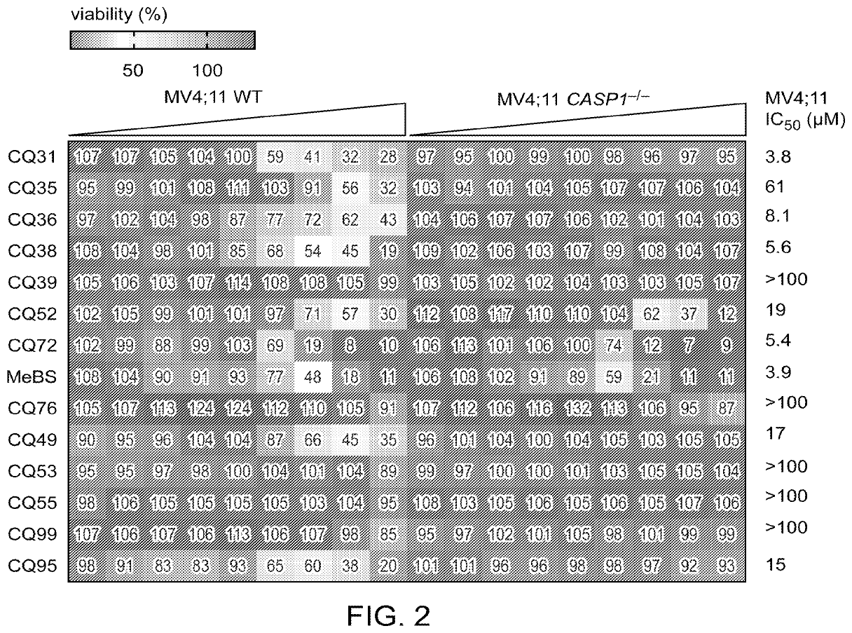

C-terminal analogs, including proline ring modifications, other natural amino acids, and carboxylic acid isosteres (Scheme 2) were also explored. Several substituents on the 4-position on the proline ring (CQ35-38) slightly reduced activity, and a cis-4-OH group (CQ39) rendered the compound completely inactive (Table 6 and FIG. 2). As expected, analogs with C-terminal amino acids other than proline (CQ52 and CQ72) were very weak PEPD and XPNPEP1 inhibitors and triggered CASP1-independent cell death similar to bestatin methyl ester (MeBs, Table 6 and FIG. 2), likely, without being bound to any theory, due to inhibition of aminopeptidases outside of the M24B family. In addition, removal of the proline carboxylate (CQ76) abrogated all activity. Analogs in which the carboxylate was replaced with amides (CQ49 and CQ95) retained similar PEPD/XPNPEP1 inhibition and cytotoxicity as CQ31, but other carboxylate isosteres (CQ53, CQ55, and CQ99) were inactive in cells. These data collectively indicated that the proline in the second position may affect activity.

TABLE 6
Inhibition of PEPD and XPNPEP1 by C-terminal analogs

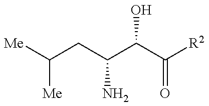

| | | | XPNPEP1 IC₅₀ |
|---|---|---|---|
| Compound | R² | PEPD IC₅₀ (μM) | (μM) |
| CQ31 | 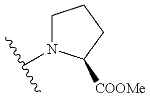 [SMILES: *N1CCC[C@H]1C(=O)OC] | 0.67 | 122 |
| CQ35 | 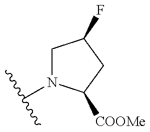 [SMILES: *N1C[C@@H](F)C[C@H]1C(=O)OC] | 0.84 | 74 |
| CQ36 | 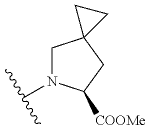 [SMILES: *N1CC2(CC2)C[C@H]1C(=O)OC] | 1.0 | 324 |
| CQ38 | 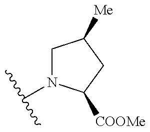 [SMILES: *N1C[C@@H](C)C[C@H]1C(=O)OC] | 2.2 | 154 |
| CQ39 | 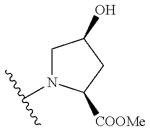 [SMILES: *N1C[C@@H](O)C[C@H]1C(=O)OC] | >100 | 190 |
| CQ76 | 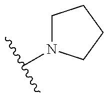 [SMILES: *N1CCCC1] | >100 | 38 |
| CQ52 | 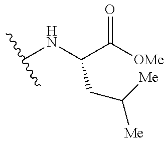 [SMILES: *N[C@@H](CC(C)C)C(=O)OC] | 51 | 57 |
| CQ72 | 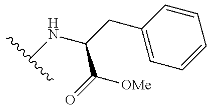 [SMILES: *N[C@@H](Cc1ccccc1)C(=O)OC] | >100 | 96 |
| CQ49 | 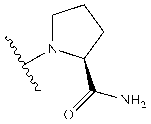 [SMILES: *N1CCC[C@H]1C(N)=O] | 0.28 | 53 |
| CQ53 | 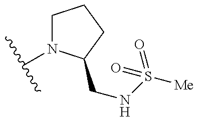 [SMILES: *N1CCC[C@H]1CNS(C)(=O)=O] | >100 | 12 |
| CQ55 | 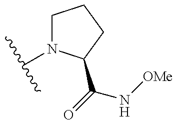 [SMILES: *N1CCC[C@H]1C(=O)NOC] | 1.6 | 303 |
| CQ99 | 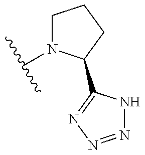 [SMILES: *N1CCC[C@H]1c1nnn[nH]1] | 6.2 | 67 |
| CQ95 | 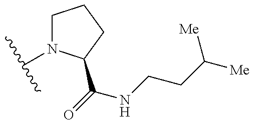 [SMILES: *N1CCC[C@H]1C(=O)NCCC(C)C] | 0.29 | 140 |

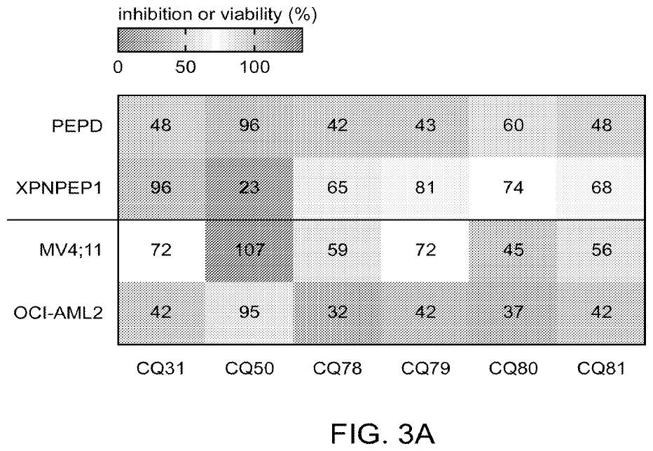

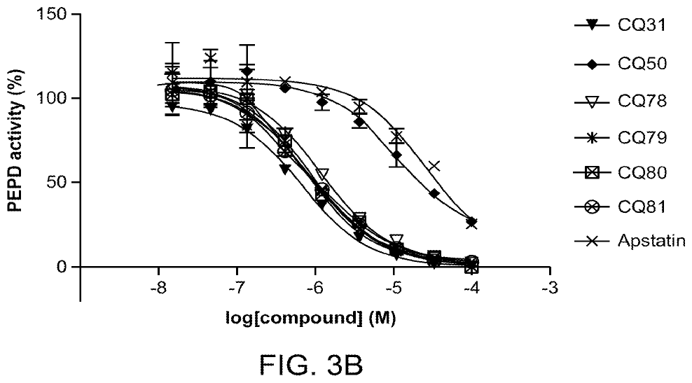

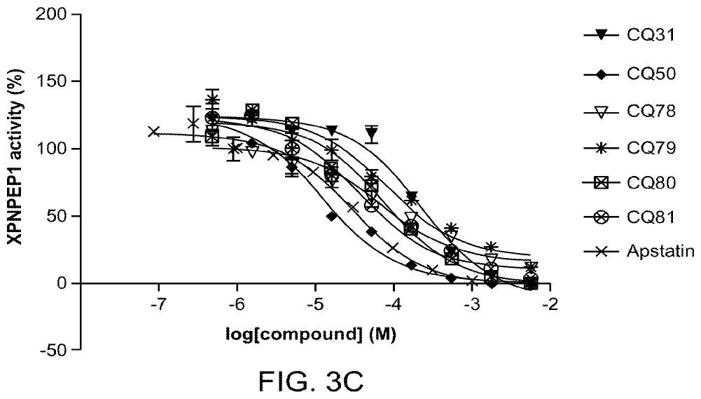

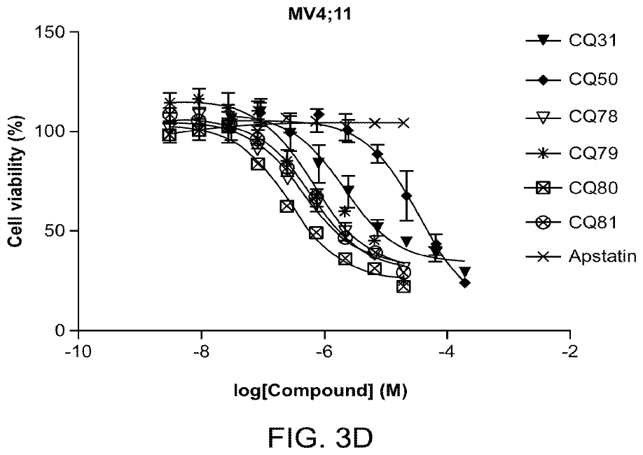

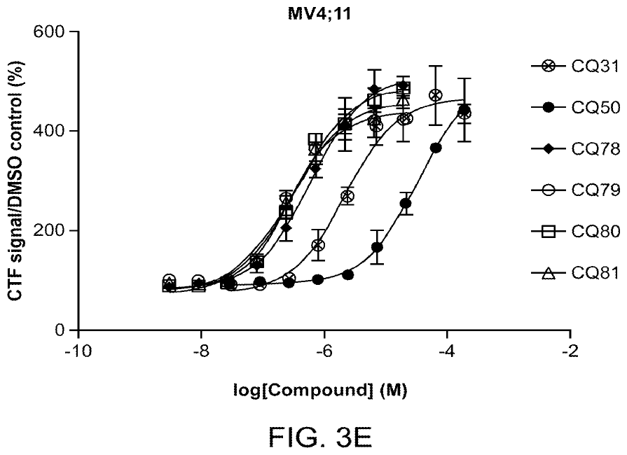

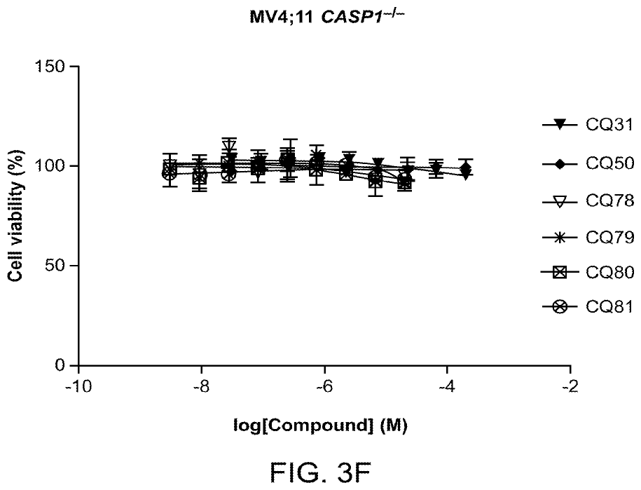

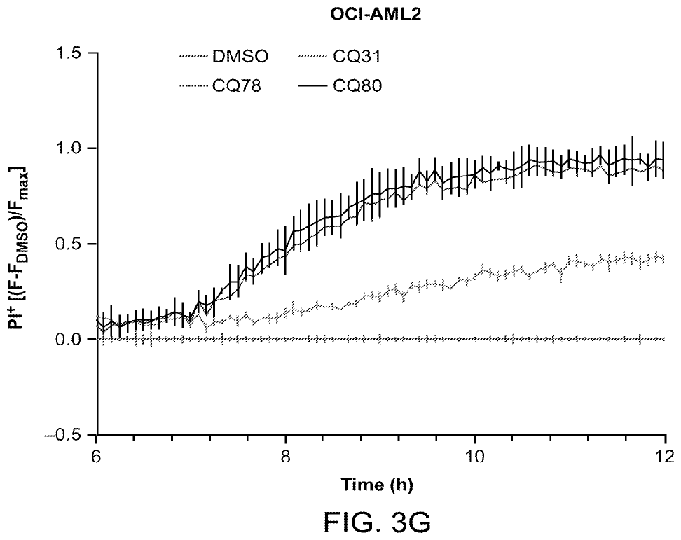

Peptide-like compounds were synthesized with the same N-terminal AHMH-Pro residues as CQ31 (Table 7). The pseudo-tetrapeptides CQ50 (AHMH-Pro-Pro-Ala-OMe) and apstatin were less potent PEPD inhibitors than CQ31 (IC₅₀s>10 μM versus 0.67 μM), but were much more potent XPNPEP1 inhibitors (IC₅₀s of 8.1 μM and 18 μM, respectively, versus 122 μM) (Table 7 and FIG. 3A-3C). Consistent with their weak PEPD inhibition, however, CQ50 only activated the CARD8 inflammasome at high doses (IC₅₀=35 μM) and apstatin was not cytotoxic at all (FIG. 3D-3E). In contrast to these pseudo-tetrapeptides, several pseudo-tripeptides (CQ78-CQ81) inhibited PEPD with potencies similar to CQ31 (IC₅₀ values˜0.7-1.2 μM), but inhibited XPNPEP1 more effectively than CQ31 (IC₅₀ values=20-50 μM) (Table 7 and FIG. 3A-3C). These pseudo-tripeptides were more cytotoxic than CQ31 to MV4;11 cells as well as OCI-AML2 cells, which also express a functional CARD8 inflammasome. In particular, CQ80 (AHMH-Pro-Leu-OMe) and CQ78 (AHMH-Pro-Val-OMe) induced CASP1-dependent cell death in MV4;11 cells with IC₅₀s of 0.27 μM and 0.45 μM, respectively, and are therefore approximately an order of magnitude more cytotoxic than CQ31 (IC₅₀=3.8 μM (Table 7 and FIG. 3D-3F). Consistent with this increased potency, CQ78 and CQ80 caused more rapid lytic cell death in OCI-AML2 cells compared to CQ31, as measured by uptake of propidium iodide (FIG. 3G). Thus, these pseudo-tripeptides, and especially CQ80, may be more effective inflammasome activators than CQ31.

TABLE 7
Inhibition of enzyme activity and cell viability by C-terminal analogs

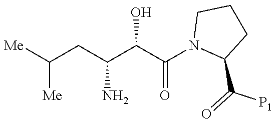

| | | | | | MV4; 11 |
|---|---|---|---|---|---|
| | | | | | CASP1^{-/-} |
| Com- | | PEPD IC₅₀ | XPNPEP1 | MV4; 11 WT | IC₅₀ |
| pound | P¹ | (μM) | IC₅₀ (μM) | IC₅₀ (μM) | (μM) |
| CQ31 | — | 0.67 | 122 | 3.8 | >100 |
| Apstatin | — | 26 | 18 | >20 | >20 |
| CQ50 | L-Pro-Ala- | 11 | 8.1 | 35 | >100 |
| | OMe | | | | |
| CQ78 | L-Val-OMe | 1.2 | 41 | 0.45 | >20 |
| CQ79 | L-Phe-OMe | 0.72 | 48 | 0.66 | >20 |
| CQ80 | L-Leu-OMe | 0.91 | 43 | 0.27 | >20 |
| CQ81 | L-Ile-OMe | 0.84 | 23 | 0.60 | >20 |

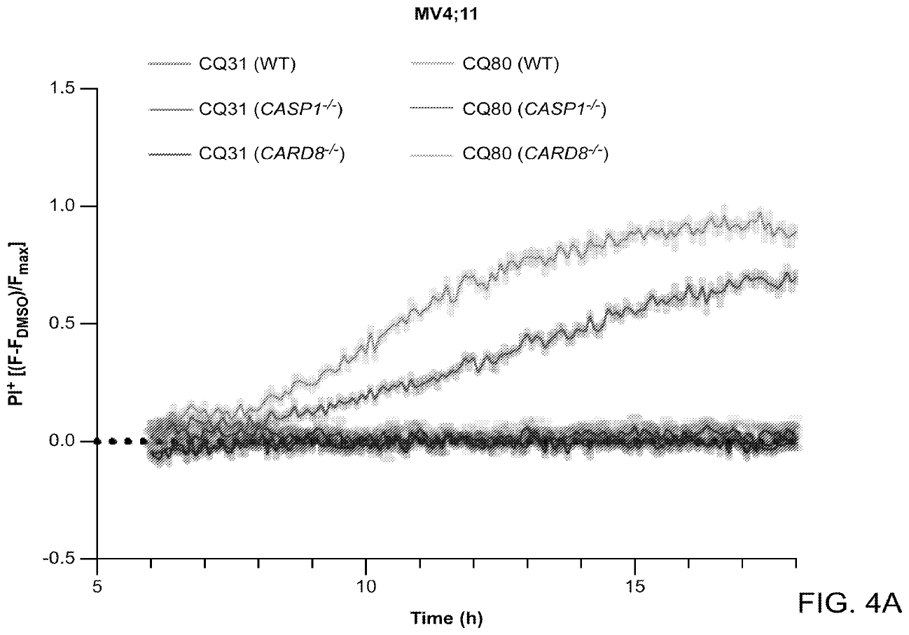

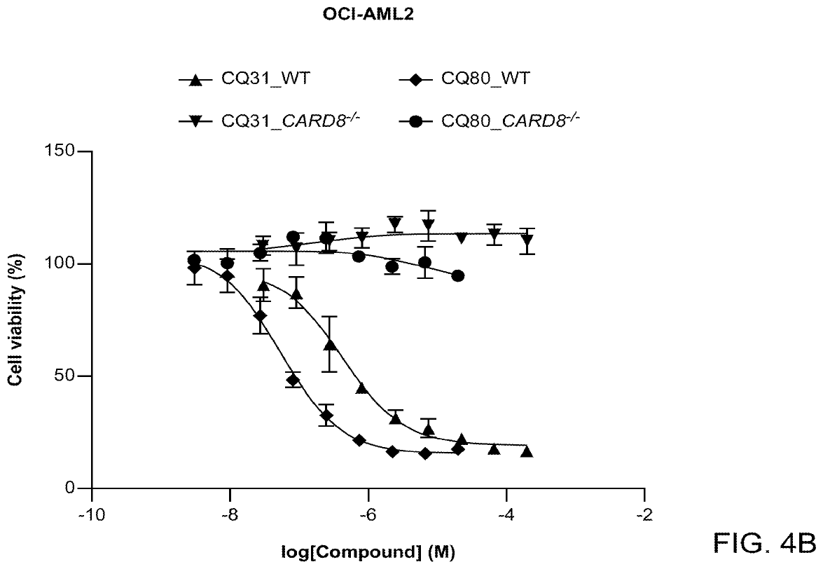

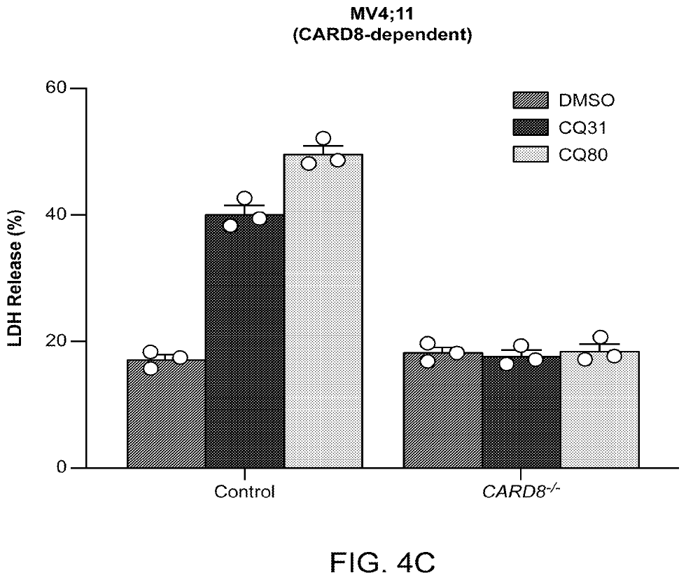

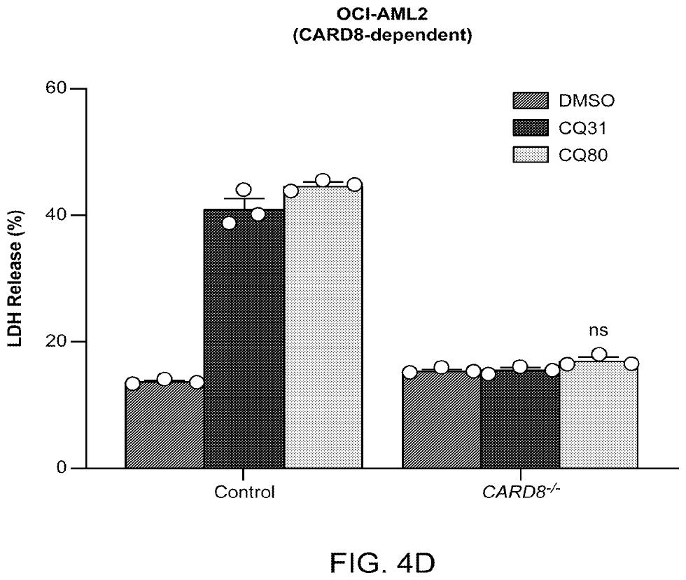

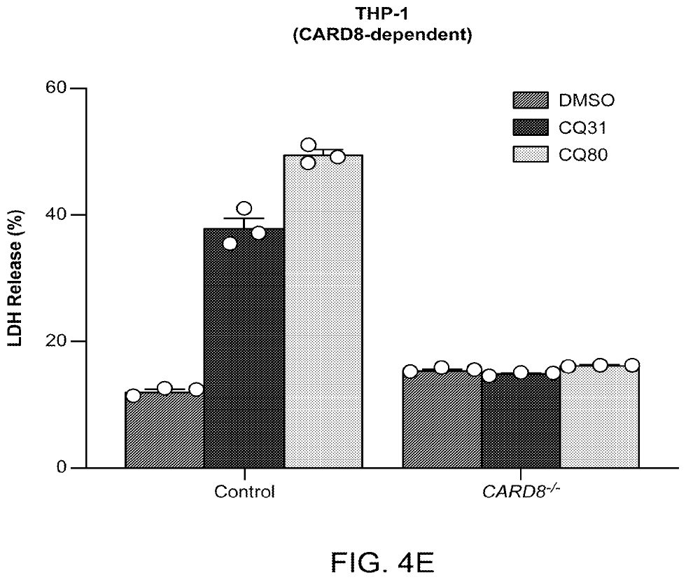

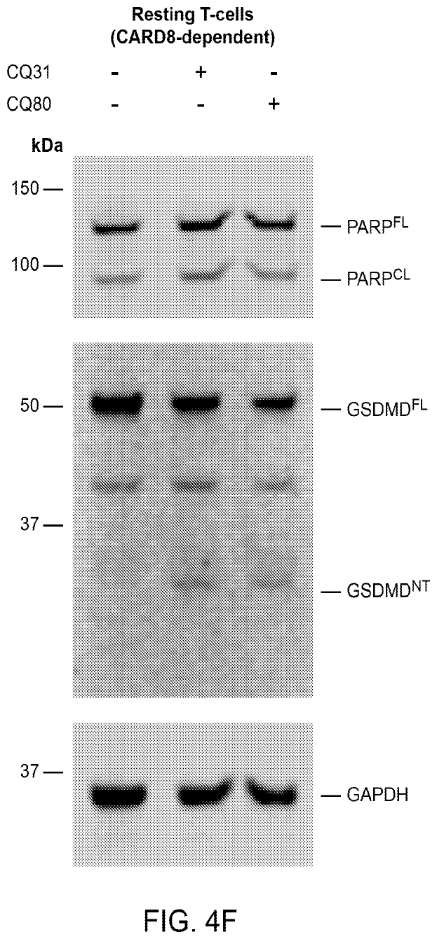

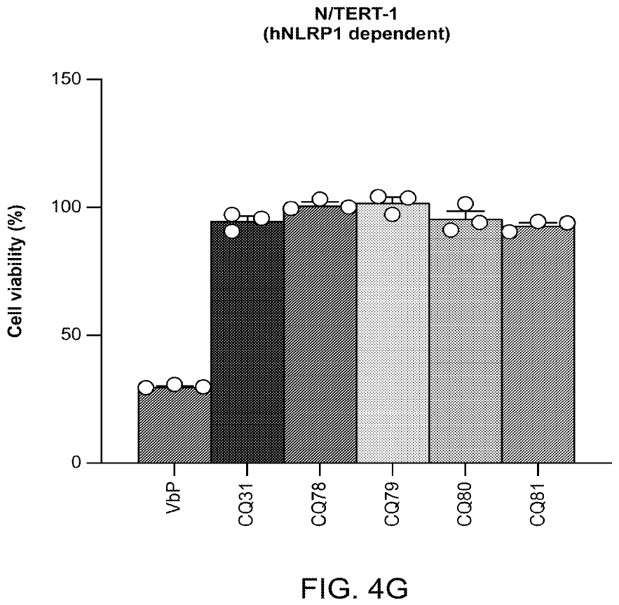

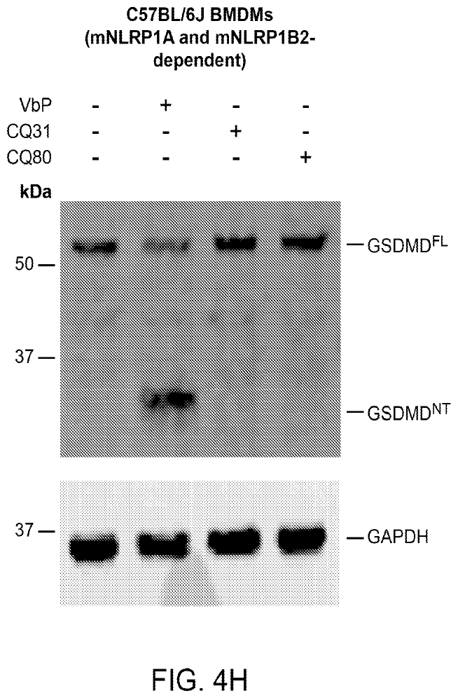

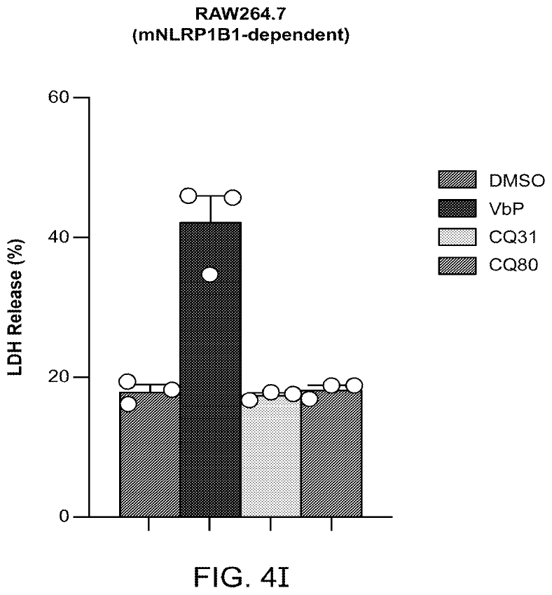

CQ80 selectively activates CARD8. Experiments were conducted to determine if CQ80 selectively activated the CARD8 inflammasome without simultaneously activating the NLRP1 inflammasome. CQ80 induced CARD8 inflammasome activation in several cell types. Both CQ31 and CQ80 induced PI uptake in WT, but not CASP1−/− or CARD8−/−, MV4;11 cells (FIG. 4A). Similarly, both CQ31 and CQ80 induced cell death in WT, but not CARD8−/−, OCI-AML2 cells (FIG. 4B). Moreover, CQ31 and CQ80 induced lactate dehydrogenase (LDH) release and GSDMD cleavage, two hallmarks of pyroptosis, in WT, but not CARD8−/−, MV4;11, OCI-AML2, and THP-1 cells (THP-1 cells also express the CARD8 inflammasome components 14) (FIG. 4C-4E). Lastly, human resting T cells are a primary cell type with a functional CARD8 inflammasome, and as expected both CQ31 and CQ80 induced GSDMD cleavage in these cells as well (FIG. 4F). Thus, CQ80 activates the CARD8 inflammasome in both cancer cell lines and normal primary cells.

Further experiments were conducted to determine if CQ80 triggered any NLRP1 inflammasome activation. Human immortalized N/TERT-1 keratinocytes express a functional human NLRP1, but not a CARD8, inflammasome. These cells were treated with VbP (which activates both NLRP1 and CARD8), CQ31, CQ80, and the other pseudo-tetrapeptides before evaluating cell viability. Only VbP induced cell death in N/TERT-1 keratinocytes (FIG. 4G). Similarly, only VbP induced pyroptosis in primary bone marrow-derived macrophages (BMDMs) (which express mouse NLRP1A and NLRP1B allele 2) and mouse RAW264.7 cells (which express mouse NLRP1B allele 1) (FIGS. 4H and 4I), as determined by LDH release and GSDMD cleavage assays. It should be noted that rodents do not have a CARD8 homolog. Overall, these data indicated that CQ80 may not activate the human or rodent NLRP1 inflammasomes.

In-vitro stability investigations. The data above demonstrate that CQ80 is an improved selective CARD8 inflammasome activator. However, its C-terminal methyl ester, which was added to improve cell penetrance, may be hydrolyzed in biological systems. Therefore, CQ80 analogs without this C-terminal methyl ester were investigated for metabolic liability.

CQ31 is completely de-esterified to form CQ04 in cell culture after 24 h. The stability of CQ31 was tested in cell culture supernatants, which may mimic cell culture. Results showed that CQ31 was rapidly converted to CQ04 within 30 min. CQ31 analogs with tert-butyl and cyclopentyl esters (CQ34 and CQ94, respectively), may be more stable to esterase-mediated hydrolysis. CQ34 was considerably more stable than CQ31 or CQ94 in cell culture supernatants, but was nevertheless completely hydrolyzed after 8 h. Interestingly, CQ31, CQ34, and CQ94 all inhibited PEPD and XPNPEP1 inhibitors similarly, but CQ34 and CQ94 were slightly more toxic to MV4;11 cells. Thus, these data suggested that increasing stability improved bioactivity.

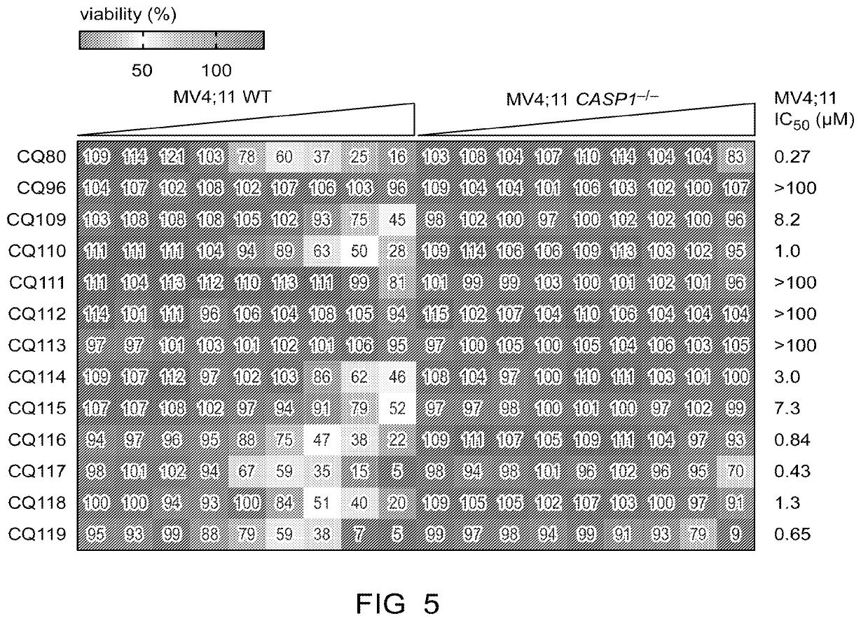

CQ80 was tested for stability in cell culture supernatants. Results indicated that CQ80 was de-esterified far more slowly than CQ31 (˜50% remained intact after 24 h), perhaps accounting for some of its increased bioactivity (FIG. 5). Amide analogs of CQ8 were synthesized (Table 8). Results indicated that several amide analogs with aliphatic or aromatic groups at the C-terminus (CQ116-119) were particularly potent XPNPEP1 inhibitors (IC₅₀ₛ<5 μm), albeit with reduced PEPD inhibitory activity. A representative of these compounds (CQ116) was completely stable in cell culture supernatants. Moreover, several of these compounds induced CASP1-dependent pyroptosis at only slightly higher concentrations as CQ80 (FIG. 5). Overall, these data indicated that CQ80 is relatively stable compared to CQ31, but that its C-terminus can be modified to remove the ester without completely abrogating its bioactivity.

TABLE 8
Inhibition of PEPD and XPNPEP1 by amide analogs of CQ80

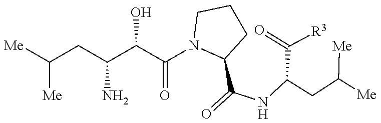

| | | | XPNPEP1 IC₅₀ |
|---|---|---|---|
| Compound | R³ | PEPD IC₅₀ (μM) | (μM) |
| CQ96 | —NH₂ | 7.5 | 10 |
| CQ109 | 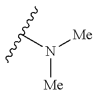 [SMILES: *N(C)C] | 4.0 | 11 |
| CQ110 | 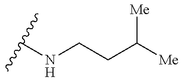 [SMILES: *NCCC(C)C] | 1.5 | 14 |
| CQ111 | 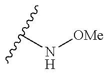 [SMILES: *NOC] | 14 | 84 |
| CQ112 | 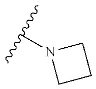 [SMILES: *N1CCC1] | 66 | 95 |
| CQ113 | 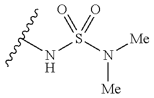 [SMILES: *NS(=O)(=O)N(C)C] | 0.56 | 2.9 |
| CQ114 | 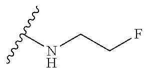 [SMILES: *NCCF] | 0.72 | 7.0 |
| CQ115 | 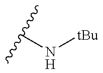 [SMILES: *NC(C)(C)C] | 3.7 | 14 |
| CQ116 | 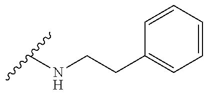 [SMILES: *NCCc1ccccc1] | 5.7 | 3.4 |
| CQ117 | 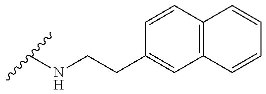 [SMILES: *NCCc1ccc2ccccc2c1] | 7.6 | 2.2 |
| CQ118 | 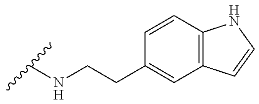 [SMILES: *NCCc1ccc2[nH]ccc2c1] | 10 | 2.0 |
| CQ119 | 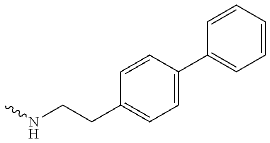 | 14 | 1.7 |

### REFERENCES

(1) Broz, P.; Dixit, V. M. Inflammasomes: mechanism of assembly, regulation and signalling. Nat. Rev. Immunol. 2016, 16 (7), 407-420. DOI: 10.1038/nri.2016.58. Rathinam, V. A.; Fitzgerald, K. A. Inflammasome Complexes: Emerging Mechanisms and Effector Functions. Cell 2016, 165 (4), 792-800. DOI: 10.1016/j.cell.2016.03.046.
(2) Ball, D. P.; Taabazuing, C. Y.; Griswold, A. R.; Orth, E. L.; Rao, S. D.; Kotliar, L. B.; Vostal, L. E.; Johnson, D. C.; Bachovchin, D. A. Caspase-1 interdomain linker cleavage is required for pyroptosis. Life Sci Alliance 2020, 3 (3). DOI: 10.26508/lsa.202000664.
(3) Robert Hollingsworth, L.; David, L.; Li, Y., Griswold, A. R.; Ruan, J.; Sharif, H.; Fontana, P.; Orth-He, E. L.; Fu, T. M.; Bachovchin, D. A.; et al. Mechanism of filament formation in UPA-promoted CARD8 and NLRP1 inflammasomes. Nat Commun 2021, 12 (1), 189. DOI: 10.1038/s41467-020-20320-y.
(4) Taabazuing, C. Y.; Griswold, A. R.; Bachovchin, D. A. The NLRP1 and CARD8 inflammasomes. Immunol. Rev. 2020. DOI: 10.1111/imr.12884.
(5) Hollingsworth, L. R.; Sharif, H.; Griswold, A. R.; Fontana, P.; Mintseris, J.; Dagbay, K. B.; Paulo, J. A.; Gygi, S. P.; Bachovchin, D. A.; Wu, H. DPP9 sequesters the C terminus of NLRP1 to repress inflammasome activation. Nature 2021, 592 (7856), 778-783. DOI: 10.1038/s41586-021-03350-4.
(6) Sharif, H.; Hollingsworth, L. R.; Griswold, A. R.; Hsiao, J. C.; Wang, Q.; Bachovchin, D. A.; Wu, H. Dipeptidyl peptidase 9 sets a threshold for CARD8 inflammasome formation by sequestering its active C-terminal fragment. Immunity 2021. DOI: 10.1016/j.immuni.2021.04.024.
(7) Rao, S. D.; Chen, Q.; Wang, Q.; Orth-He, E. L.; Saoi, M.; Griswold, A. R.; Bhattacharjee, A.; Ball, D. P.; Huang, H. C.; Chui, A. J.; et al. M24B aminopeptidase inhibitors selectively activate the CARD8 inflammasome. Nat. Chem. Biol. 2022. DOI: 10.1038/s41589-021-00964-7.
(8) Finger, J. N.; Lich, J. D.; Dare, L. C.; Cook, M. N.; Brown, K. K.; Duraiswami, C.; Bertin, J.; Gough, P. J. Autolytic proteolysis within the function to find domain (FIIND) is required for NLRP1 inflammasome activity. J. Biol. Chem. 2012, 287 (30), 25030-25037. DOI: 10.1074/jbc.M112.378323. D'Osualdo, A.; Weichenberger, C. X.; Wagner, R. N.; Godzik, A.; Wooley, J.; Reed, J. C. CARD8 and NLRP1 undergo autoproteolytic processing through a ZU5-like domain. PLoS One 2011, 6 (11), e27396. DOI: 10.1371/journal.pone.0027396.
(9) Chui, A. J.; Okondo, M. C.; Rao, S. D.; Gai, K.; Griswold, A. R.; Johnson, D. C.; Ball, D. P.; Taabazuing, C. Y.; Orth, E. L.; Vittimberga, B. A.; et al. N-terminal degradation activates the NLRP1B inflammasome. Science 2019, 364 (6435), 82-85. DOI: 10.1126/science.aau1208.
(10) Sandstrom, A.; Mitchell, P. S.; Goers, L.; Mu, E. W.; Lesser, C. F.; Vance, R. E. Functional degradation: A mechanism of NLRP1 inflammasome activation by diverse pathogen enzymes. Science 2019, (6435). DOI: 10.1126/science.aau1330.
(11) Huang, M.; Zhang, X.; Toh, G. A.; Gong, Q.; Wang, J.; Han, Z.; Wu, B.; Zhong, F.; Chai, J. Structural and biochemical mechanisms of NLRP1 inhibition by DPP9. Nature 2021, 592 (7856), 773-777. DOI: 10.1038/s41586-021-03320-w.
(12) Okondo, M. C.; Johnson, D. C.; Sridharan, R.; Go, E. B.; Chui, A. J.; Wang, M. S.; Poplawski, S. E.; Wu, W.; Liu, Y.; Lai, J. H.; et al. DPP8 and DPP9 inhibition induces pro-caspase-1-dependent monocyte and macrophage pyroptosis. Nat. Chem. Biol. 2017, 13 (1), 46-53. DOI: 10.1038/nchembio.2229. Okondo, M. C.; Rao, S. D.; Taabazuing, C. Y.; Chui, A. J.; Poplawski, S. E.; Johnson, D. C.; Bachovchin, D. A. Inhibition of Dpp8/9 Activates the Nlrp1b Inflammasome. Cell Chem Biol 2018, 25 (3), 262-267 e265. DOI: 10.1016/j.chembiol.2017.12.013. Griswold, A. R.; Ball, D. P.; Bhattacharjee, A.; Chui, A. J.; Rao, S. D.; Taabazuing, C. Y.; Bachovchin, D. A. DPP9's Enzymatic Activity and Not Its Binding to CARD8 Inhibits Inflammasome Activation. ACS Chem. Biol. 2019, 14 (11), 2424-2429. DOI: 10.1021/acschembio.9b00462.
(13) Zhong, F. L.; Robinson, K.; Teo, D. E. T.; Tan, K. Y.; Lim, C.; Harapas, C. R.; Yu, C. H.; Xie, W. H.; Sobota, R. M.; Au, V. B.; et al. Human DPP9 represses NLRP1 inflammasome and protects against autoinflammatory diseases via both peptidase activity and FUND domain binding. J. Biol. Chem. 2018, 293 (49), 18864-18878. DOI: 10.1074/jbc.RA 118.004350.
(14) Johnson, D. C.; Taabazuing, C. Y.; Okondo, M. C.; Chui, A. J.; Rao, S. D.; Brown, F. C.; Reed, C.; Peguero, E.; de Stanchina, E.; Kentsis, A.; et al. DPP8/DPP9 inhibitor-induced pyroptosis for treatment of acute myeloid leukemia. Nat. Med. 2018, 24 (8), 1151-1156. DOI: 10.1038/s41591-018-0082-y.
(15) Tang, H. K.; Tang, H. Y.; Hsu, S. C.; Chu, Y. R.; Chien, C. H.; Shu, C. H.; Chen, X. Biochemical properties and expression profile of human prolyl dipeptidase DPP9. Arch. Biochem. Biophys. 2009, 485 (2), 120-127. DOI: 10.1016/j.abb.2009.02.015. Geiss-Friedlander, R.; Parmentier, N.; Moller, U.; Urlaub, H.; Van den Eynde, B. J.; Melchior, F. The cytoplasmic peptidase DPP9 is rate-limiting for degradation of proline-containing peptides. J. Biol. Chem. 2009, 284 (40), 27211-27219. DOI: 10.1074/jbc.M109.041871. Griswold, A. R.; Cifani, P.; Rao, S. D.; Axelrod, A. J.; Miele, M. M.; Hendrickson, R. C.; Kentsis, A.; Bachovchin, D. A. A Chemical Strategy for Protease Substrate Profiling. Cell Chem Biol 2019, 26 (6), 901-907 e906. DOI: 10.1016/j.chembiol.2019.03.007.
(16) Lupi, A.; Tenni, R.; Rossi, A.; Cetta, G.; Forlino, A. Human prolidase and prolidase deficiency: an overview on the characterization of the enzyme involved in proline recycling and on the effects of its mutations. Amino Acids 2008, 35 (4), 739-752. DOI: 10.1007/s00726-008-0055-4.
(17) Wilk, P.; Uehlein, M.; Kalms, J.; Dobbek, H.; Mueller, U.; Weiss, M. S. Substrate specificity and reaction mechanism of human prolidase. FEBS J 2017, 284 (17), 2870-2885. DOI: 10.1111/febs.14158. Singh, R.; Jamdar, S. N.; Goyal, V. D.; Kumar, A.; Ghosh, B.; Makde, R. D. Structure of the human aminopeptidase XPNPEP3 and comparison of its in vitro activity with Icp55 orthologs: Insights into diverse cellular processes. J. Biol. Chem. 2017, 292 (24), 10035-10047. DOI: 10.1074/jbc.M117.783357. Maggiora, L. L.; Orawski, A. T.; Simmons, W. H. Apstatin analogue inhibitors of aminopeptidase P, a bradykinin-degrading enzyme. J. Med. Chem. 1999, 42 (13), 2394-2402. DOI: 10.1021/jm9805642.
(18) Wang, Y.; He, Q. F.; Wang, H. W.; Zhou, X.; Huang, Z. Y.; Qin, Y. Highly diastereoselective enolate addition of O-protected alpha-hydroxyacetate to (S(R))-tert-butanesulfinylimines: synthesis of taxol side chain. J. Org. Chem. 2006, 71 (4), 1588-1591. DOI: 10.1021/jo052298n. Ke, B.; Qin, Y.; Zhao, F.; Qu, Y. Synthesis and biological evaluation of novel 3′-N-tert-butylsulfonyl analogues of docetaxel. Bioorg. Med. Chem. Lett. 2008, 18 (17), 4783-4785. DOI: 10.1016/j.bmcl.2008.07.101.
(19) Prechel, M. M.; Orawski, A. T.; Maggiora, L. L.; Simmons, W. H. Effect of a new aminopeptidase P inhibitor, apstatin, on bradykinin degradation in the rat lung. J. Pharmacol. Exp. Ther. 1995, 275 (3), 1136-1142.
(20) Johnson, D. C.; Okondo, M. C.; Orth, E. L.; Rao, S. D.; Huang, H. C.; Ball, D. P.; Bachovchin, D. A. DPP8/9 inhibitors activate the CARD8 inflammasome in resting lymphocytes. Cell Death Dis. 2020, 11 (8), 628. DOI: 10.1038/s41419-020-02865-4. Linder, A.; Bauernfried, S.; Cheng, Y.; Albanese, M.; Jung, C.; Keppler, O. T.; Hornung, V. CARD8 inflammasome activation triggers pyroptosis in human T cells. EMBO J. 2020, e105071. DOI: 10.15252/embj.2020105071.
(21) Zhong, F. L.; Mamai, O.; Sborgi, L.; Boussofara, L.; Hopkins, R.; Robinson, K.; Szeverenyi, I.; Takeichi, T.; Balaji, R.; Lau, A.; et al. Germline NLRP1 Mutations Cause Skin Inflammatory and Cancer Susceptibility Syndromes via Inflammasome Activation. Cell 2016, 167 (1), 187-202 e117. DOI: 10.1016/j.cell.2016.09.001.
(22) Gai, K.; Okondo, M. C.; Rao, S. D.; Chui, A. J.; Ball, D. P.; Johnson, D. C.; Bachovchin, D. A. DPP8/9 inhibitors are universal activators of functional NLRP1 alleles. Cell Death Dis. 2019, 10 (8), 587. DOI: 10.1038/s41419-019-1817-5.
(23) Orth-He, E. L.; Huang, H.-C.; Rao, S. D.; Wang, Q.; Chen, Q.; O'Mara, C. M.; Chui, A. J.; Saoi, M.; Griswold, A. R.; Bhattacharjee, A.; et al. Cytosolic peptide accumulation activates the NLRP1 and CARD8 inflammasomes. bioRxiv 2022, 2022.2003.2022.485298. DOI: 10.1101/2022.03.22.485298.
(24) Wang, Q.; Hsiao, J. C.; Yardeny, N.; Huang, H.-C.; O'Mara, C. M.; Orth-He, E. L.; Ball, D. P.; Bachovchin, D. A. The NLRP1 and CARD8 inflammasomes detect reductive stress. bioRxiv 2022, 2022.2003.2022.485209. DOI: 10.1101/2022.03.22.485209.
(25) Sharif, H.; Hollingsworth, L. R.; Griswold, A. R.; Hsiao, J. C.; Wang, Q.; Bachovchin, D. A.; Wu, H. Structural mechanism of CARD8 regulation by DPP9. bioRxiv 2021, 2021.2001.2013.426575. DOI: 10.1101/2021.01.13.426575.
(26) Chui, A. J.; Griswold, A. R.; Taabazuing, C. Y.; Orth, E. L.; Gai, K.; Rao, S. D.; Ball, D. P.; Hsiao, J. C.; Bachovchin, D. A. Activation of the CARD8 Inflammasome Requires a Disordered Region. Cell Rep 2020, 33 (2), 108264. DOI: 10.1016/j.celrep.2020.108264.

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:

A. A compound according to Formula I

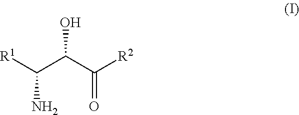

or a pharmaceutically acceptable salt and/or solvate thereof; wherein
R¹ is C₁-C₆ alkyl, —(CH₂)ₙ—(R⁴), cycloalkyl, adamantly, heterocyclyl, aryl, aralkyl, or heteroaryl;
n is 1, 2, 3, 4, 5, or 6;
R² is a heterocyclyl, alkylamino carboxylate alkyl ester, or aralkylamino carboxylate alkyl ester, optionally where R² is heterocyclyl substituted with one or more of carboxylate alkyl ester, hydroxide, halogen, cyclo, alkyl, amide, alkylamido, alkylcarbamoyl, alkylsulfonamido, tetrazole, carbonyl amino acid, or carboxy alkylamido; and
R⁴ is trifluoromethyl or hydroxide.
   B. The compound of Paragraph A, wherein R¹ is a branched C₁-C₆ alkyl.
   C. The compound of Paragraph A or Paragraph B, wherein R¹ is

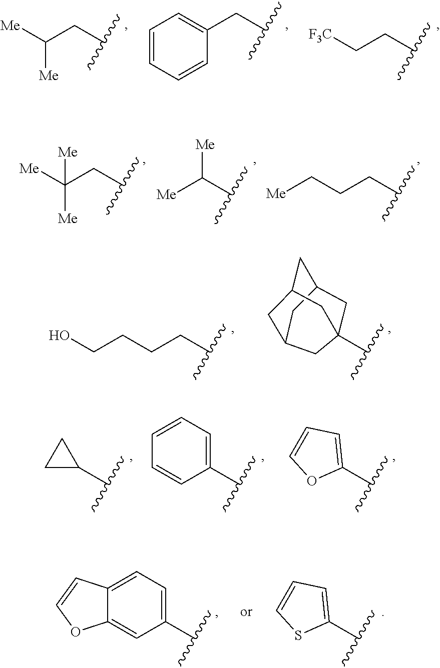

D. The compound of any one of Paragraphs A-C, wherein R¹ is

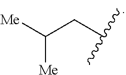 [SMILES: *CC(C)C]

E. The compound of any one of Paragraphs A-D, wherein R² is

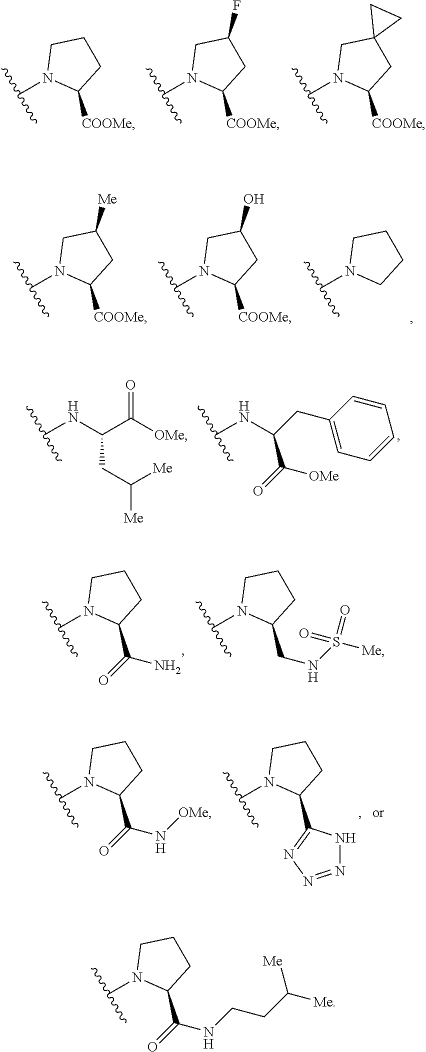

F. The compound of any one of Paragraphs A-D, wherein R² is

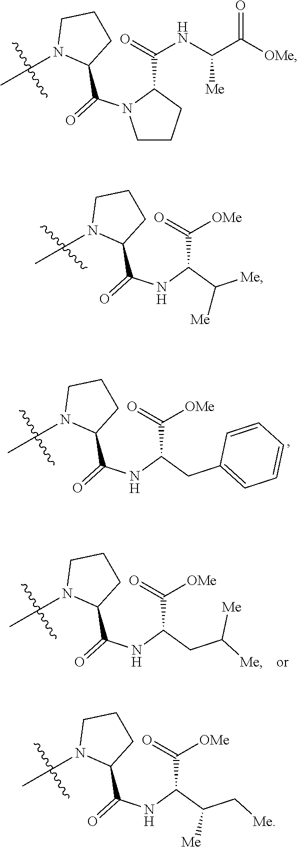 [SMILES: *N1CCC[C@H]1C(=O)N1CCC[C@H]1C(=O)N[C@@H](C)C(=O)OC ; *N1CCC[C@H]1C(=O)N[C@H](C(=O)OC)C(C)C ; *N1CCC[C@H]1C(=O)N[C@@H](Cc1ccccc1)C(=O)OC ; *N1CCC[C@H]1C(=O)N[C@@H](CC(C)C)C(=O)OC ; *N1CCC[C@H]1C(=O)N[C@H](C(=O)OC)[C@@H](C)CC]

G. The compound of any one of Paragraphs A-D, wherein R² is

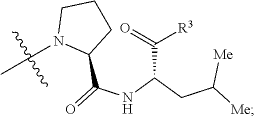

wherein R³ is NH₂, alkylamino, N(Me)₂, N(H)OMe, heterocyclyl, sulfonamido, fluoroalkyl amino, aralkyl amino, or heteroarylalkyl amino.
   H. The compound of Paragraph G, wherein R³ is aralkyl amino or heteroarylalkyl amino.
   I. The compound of Paragraph G, wherein R³ is NH₂, N(Me)₂,

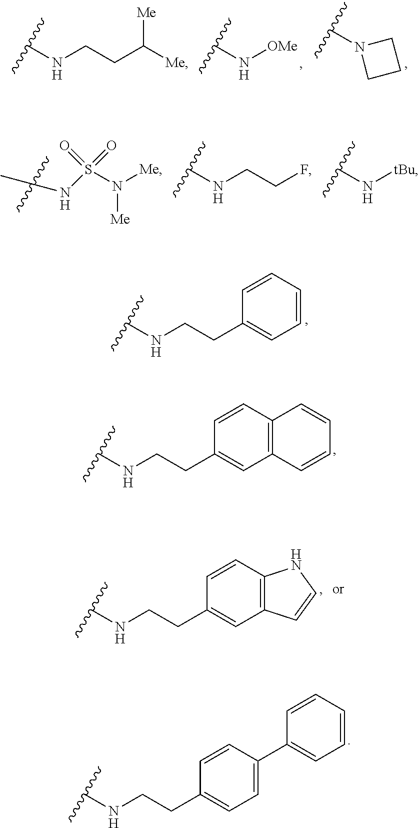

J. The compound of Paragraph A, wherein the compound is

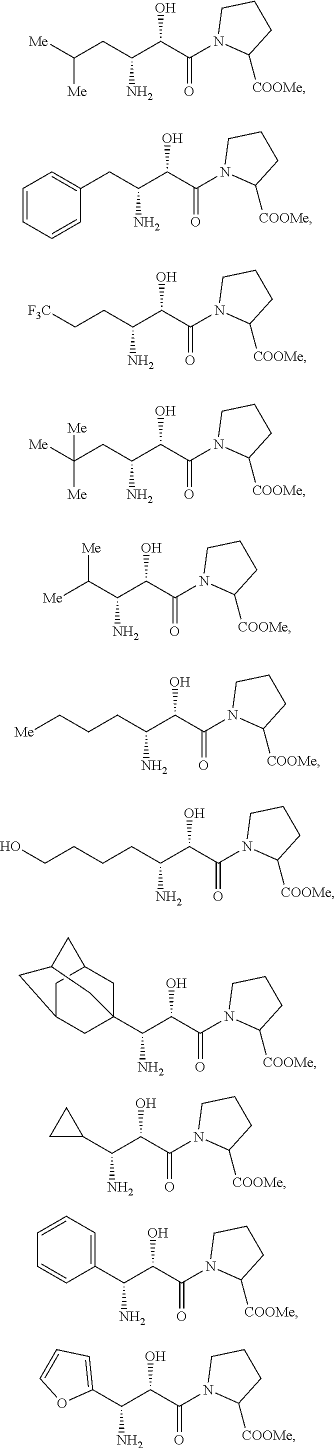

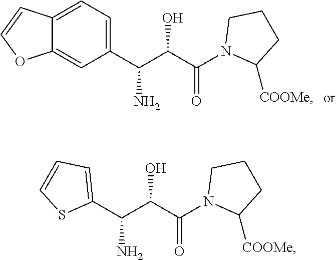 [SMILES: COC(=O)C1CCCN1C(=O)[C@@H](O)[C@H](N)c1ccc2ccoc2c1 ; COC(=O)C1CCCN1C(=O)[C@@H](O)[C@H](N)c1cccs1]

or a pharmaceutically acceptable salt and/or solvate thereof.
K. The compound of Paragraph A, wherein the compound is

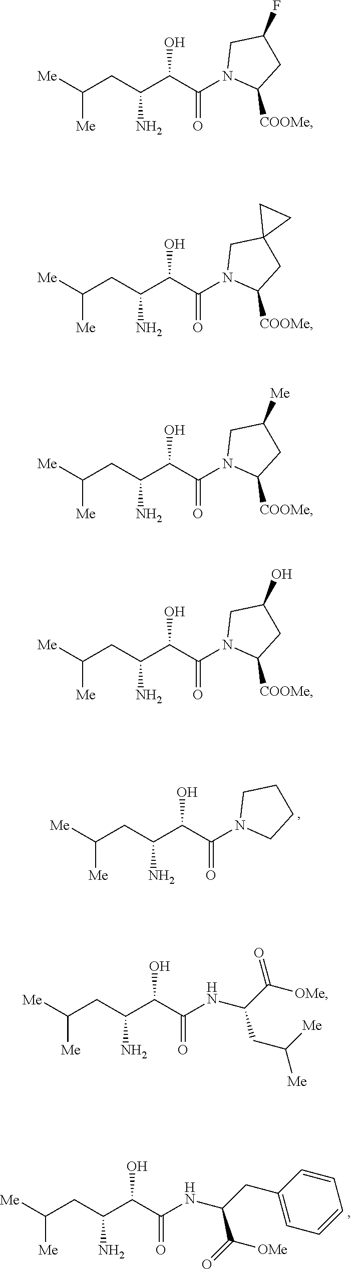

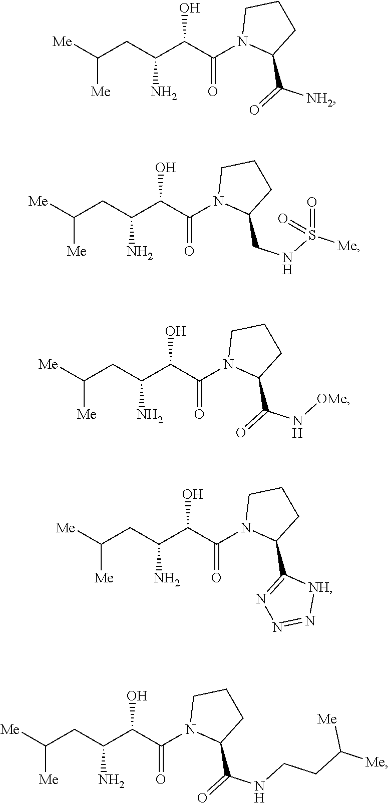 [SMILES: CC(C)C[C@@H](N)[C@H](O)C(=O)N1CCC[C@H]1C(N)=O ; CC(C)C[C@@H](N)[C@H](O)C(=O)N1CCC[C@H]1CNS(C)(=O)=O ; CONC(=O)[C@@H]1CCCN1C(=O)[C@@H](O)[C@H](N)CC(C)C ; CC(C)C[C@@H](N)[C@H](O)C(=O)N1CCC[C@H]1c1nnn[nH]1 ; CC(C)CCNC(=O)[C@@H]1CCCN1C(=O)[C@@H](O)[C@H](N)CC(C)C]

or a pharmaceutically acceptable salt and/or solvate thereof.
L. The compound of Paragraph A, wherein the compound is

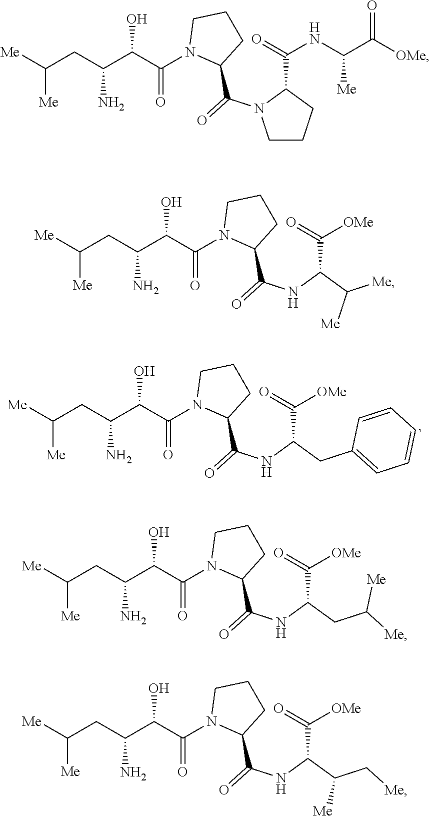 [SMILES: COC(=O)[C@H](C)NC(=O)[C@@H]1CCCN1C(=O)[C@@H]1CCCN1C(=O)[C@@H](O)[C@H](N)CC(C)C ; COC(=O)[C@@H](NC(=O)[C@@H]1CCCN1C(=O)[C@@H](O)[C@H](N)CC(C)C)C(C)C ; COC(=O)[C@H](Cc1ccccc1)NC(=O)[C@@H]1CCCN1C(=O)[C@@H](O)[C@H](N)CC(C)C ; COC(=O)[C@H](CC(C)C)NC(=O)[C@@H]1CCCN1C(=O)[C@@H](O)[C@H](N)CC(C)C ; CC[C@H](C)[C@H](NC(=O)[C@@H]1CCCN1C(=O)[C@@H](O)[C@H](N)CC(C)C)C(=O)OC]

or a pharmaceutically acceptable salt and/or solvate thereof.
   M. The compound of Paragraph A, wherein the compound is

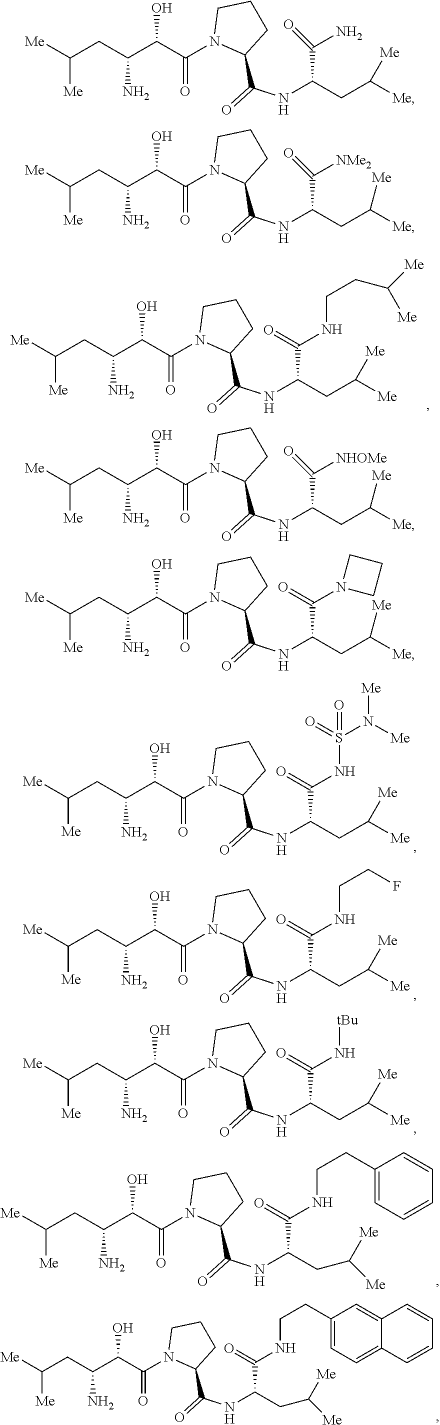

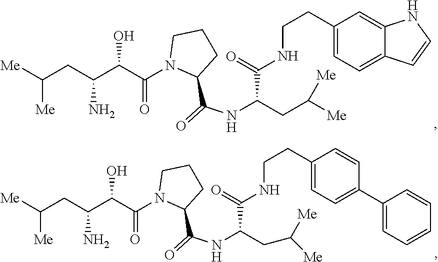

or a pharmaceutically acceptable salt and/or solvate thereof.

Other embodiments are set forth in the following claims, along with the full scope of equivalents to which such claims are entitled.

1. A compound according to Formula I 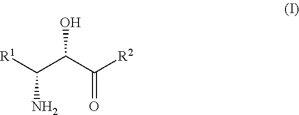
or a pharmaceutically acceptable salt and/or solvate thereof; wherein
R¹ is C₁-C₆ alkyl, —(CH₂)ₙ—(R⁴), cycloalkyl, adamantly, heterocyclyl, aryl, aralkyl, or heteroaryl;
n is 1, 2, 3, 4, 5, or 6;
R² is a heterocyclyl, alkylamino carboxylate alkyl ester, or aralkylamino carboxylate alkyl ester, optionally where R² is heterocyclyl substituted with one or more of carboxylate alkyl ester, hydroxide, halogen, cyclo, alkyl, amide, alkylamido, alkylcarbamoyl, alkylsulfonamido, tetrazole, carbonyl amino acid, or carboxy alkylamido; and
R⁴ is trifluoromethyl or hydroxide.

2. The compound of claim 1, wherein R¹ is a branched C₁-C₆ alkyl.

3. The compound of claim 1, wherein R¹ is

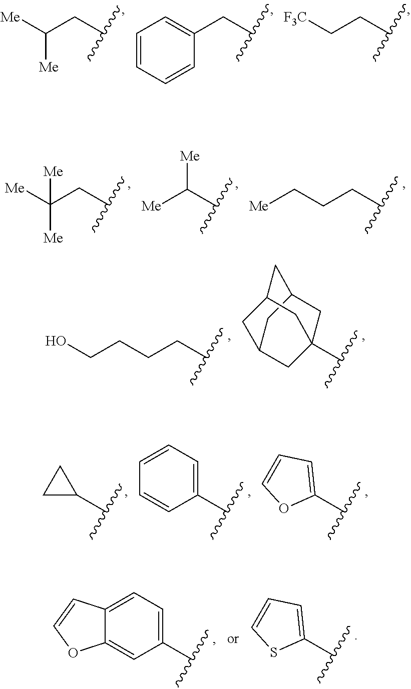

4. The compound of claim 1, wherein R¹ is

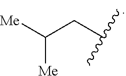 [SMILES: *CC(C)C]

5. The compound of claim 1, wherein R² is

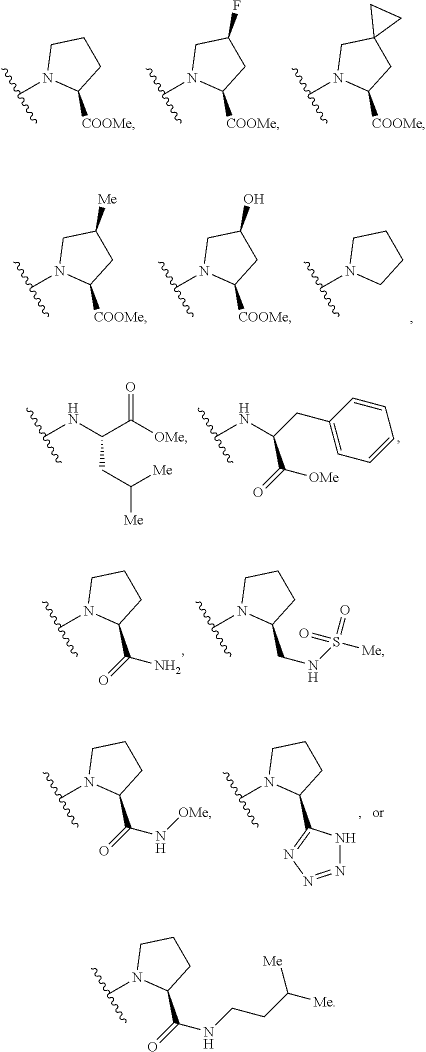

6. The compound of claim 1, wherein R² is

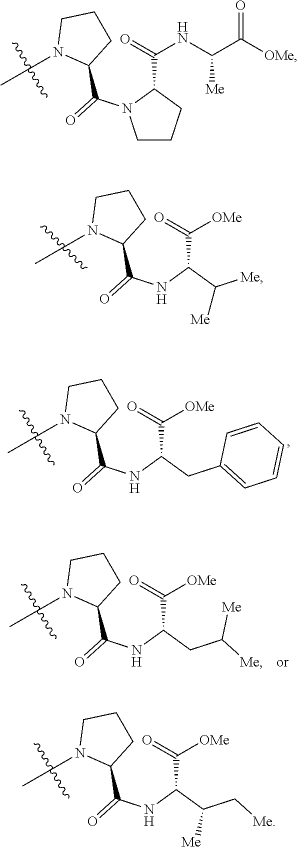 [SMILES: *N1CCC[C@H]1C(=O)N1CCC[C@H]1C(=O)N[C@@H](C)C(=O)OC ; *N1CCC[C@H]1C(=O)N[C@H](C(=O)OC)C(C)C ; *N1CCC[C@H]1C(=O)N[C@@H](Cc1ccccc1)C(=O)OC ; *N1CCC[C@H]1C(=O)N[C@@H](CC(C)C)C(=O)OC ; *N1CCC[C@H]1C(=O)N[C@H](C(=O)OC)[C@@H](C)CC]

7. The compound of claim 1,
wherein R² is 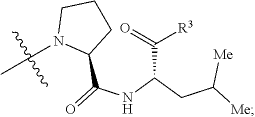
and
wherein R³ is NH₂, alkylamino, N(Me)₂, N(H)OMe, heterocyclyl, sulfonamido, fluoroalkyl amino, aralkyl amino, or heteroarylalkyl amino.

8. The compound of claim 7, wherein R³ is aralkyl amino or heteroarylalkyl amino.

9. The compound of claim 7, wherein R³ is NH₂, N(Me)₂,

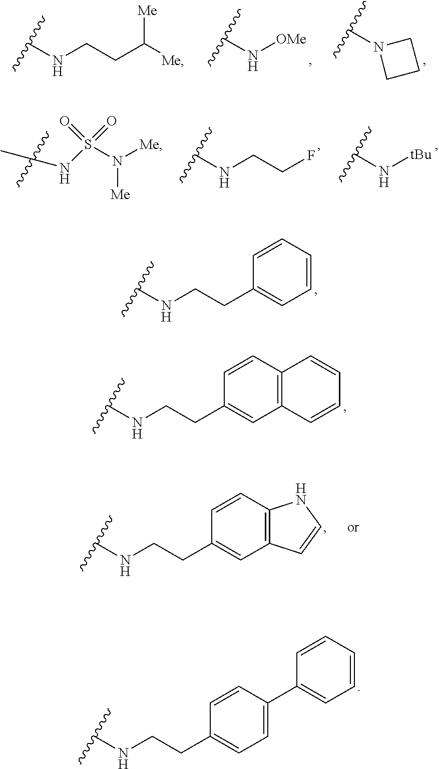

10. The compound of claim 1, wherein the compound is 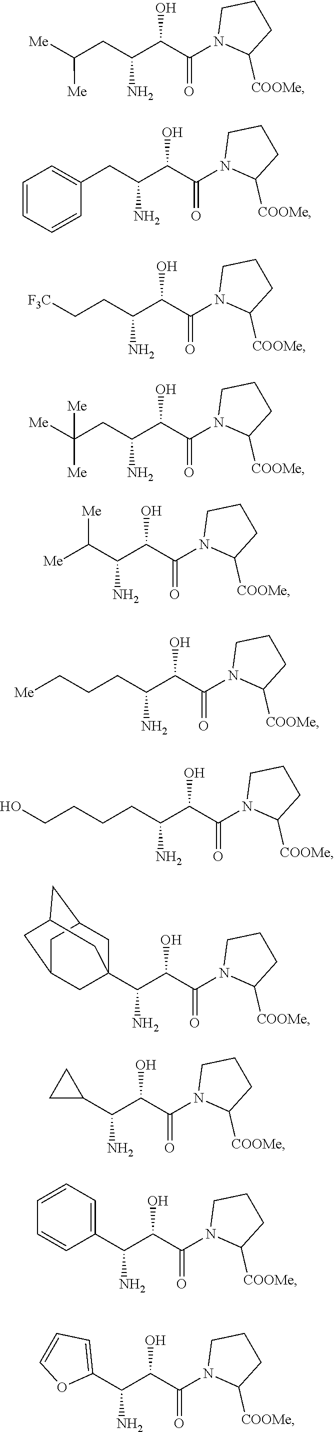 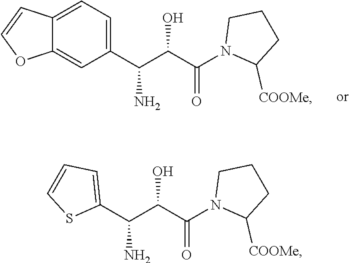 [SMILES: COC(=O)C1CCCN1C(=O)[C@@H](O)[C@H](N)c1ccc2ccoc2c1 ; COC(=O)C1CCCN1C(=O)[C@@H](O)[C@H](N)c1cccs1]
or a pharmaceutically acceptable salt and/or solvate thereof.

11. The compound of claim 1, wherein the compound is 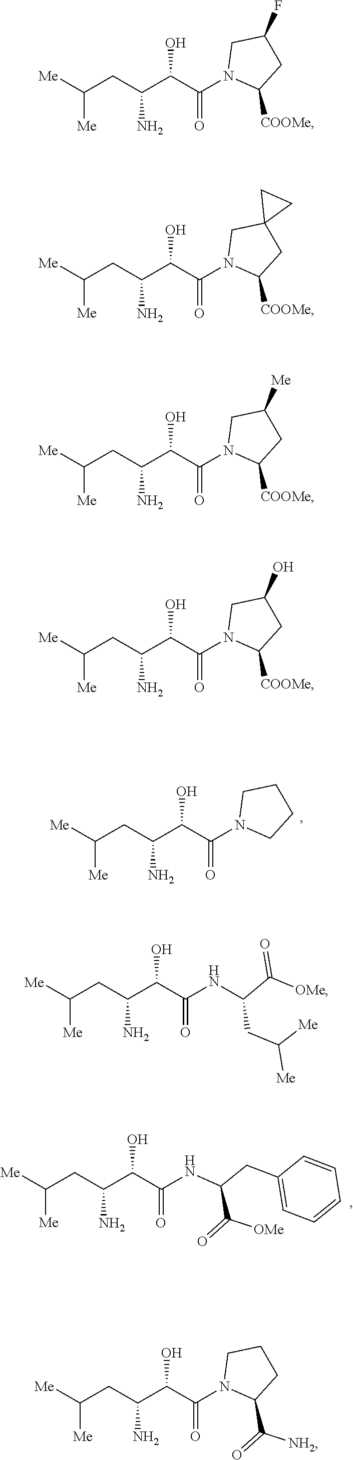 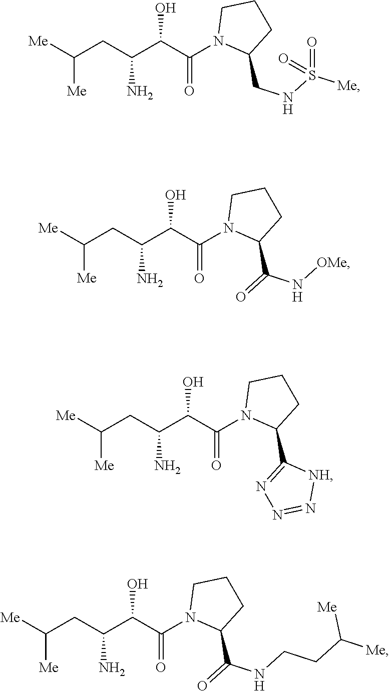 [SMILES: CC(C)C[C@@H](N)[C@H](O)C(=O)N1CCC[C@H]1CNS(C)(=O)=O ; CONC(=O)[C@@H]1CCCN1C(=O)[C@@H](O)[C@H](N)CC(C)C ; CC(C)C[C@@H](N)[C@H](O)C(=O)N1CCC[C@H]1c1nnn[nH]1 ; CC(C)CCNC(=O)[C@@H]1CCCN1C(=O)[C@@H](O)[C@H](N)CC(C)C]
or a pharmaceutically acceptable salt and/or solvate thereof.

12. The compound of claim 1, wherein the compound is 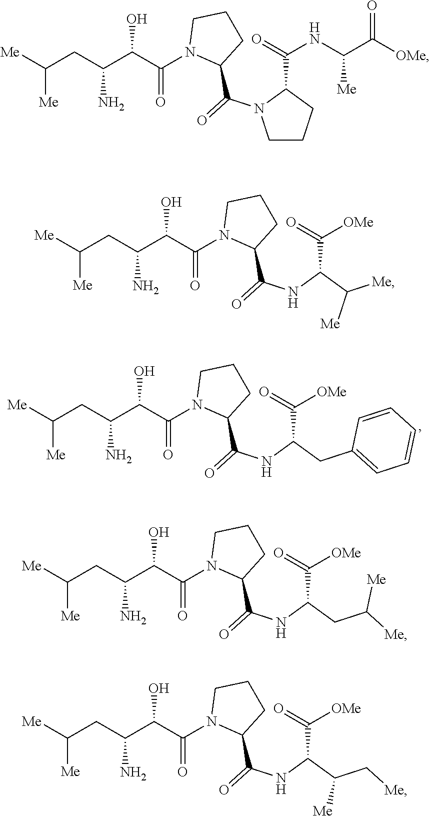 [SMILES: COC(=O)[C@H](C)NC(=O)[C@@H]1CCCN1C(=O)[C@@H]1CCCN1C(=O)[C@@H](O)[C@H](N)CC(C)C ; COC(=O)[C@@H](NC(=O)[C@@H]1CCCN1C(=O)[C@@H](O)[C@H](N)CC(C)C)C(C)C ; COC(=O)[C@H](Cc1ccccc1)NC(=O)[C@@H]1CCCN1C(=O)[C@@H](O)[C@H](N)CC(C)C ; COC(=O)[C@H](CC(C)C)NC(=O)[C@@H]1CCCN1C(=O)[C@@H](O)[C@H](N)CC(C)C ; CC[C@H](C)[C@H](NC(=O)[C@@H]1CCCN1C(=O)[C@@H](O)[C@H](N)CC(C)C)C(=O)OC]
or a pharmaceutically acceptable salt and/or solvate thereof.

13. The compound of claim 1, wherein the compound is 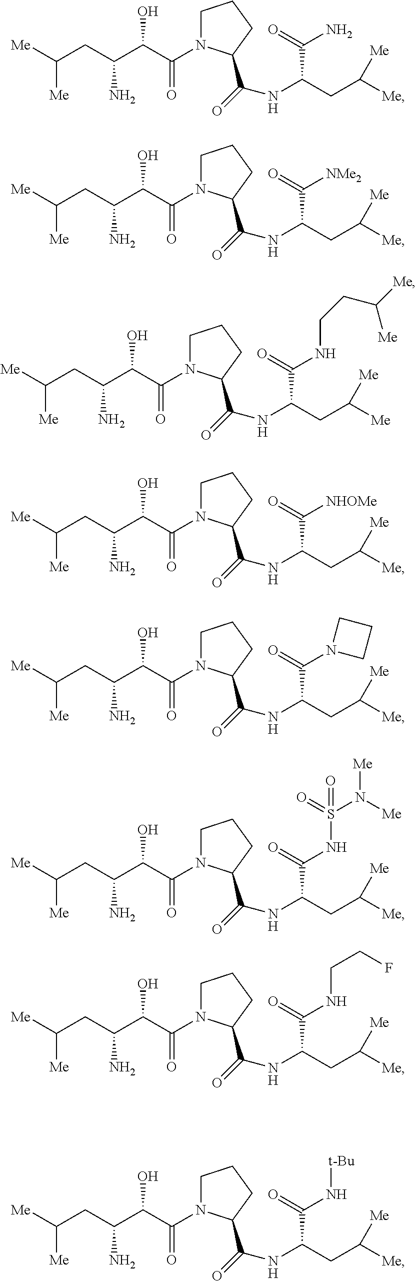 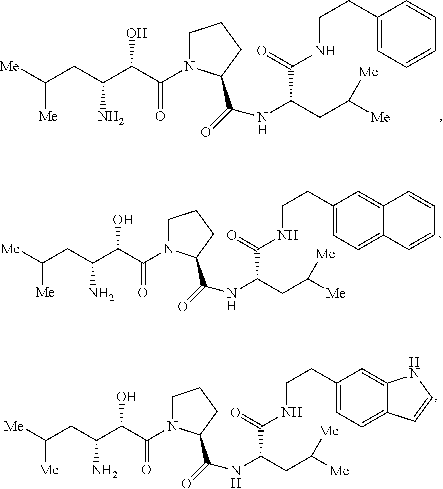 [SMILES: CC(C)C[C@H](NC(=O)[C@@H]1CCCN1C(=O)[C@@H](O)[C@H](N)CC(C)C)C(=O)NCCc1ccccc1 ; CC(C)C[C@H](NC(=O)[C@@H]1CCCN1C(=O)[C@@H](O)[C@H](N)CC(C)C)C(=O)NCCc1ccc2ccccc2c1 ; CC(C)C[C@H](NC(=O)[C@@H]1CCCN1C(=O)[C@@H](O)[C@H](N)CC(C)C)C(=O)NCCc1ccc2cc[nH]c2c1] 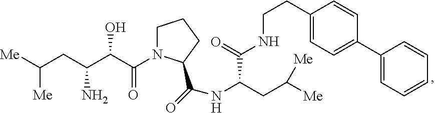 [SMILES: CC(C)C[C@H](NC(=O)[C@@H]1CCCN1C(=O)[C@@H](O)[C@H](N)CC(C)C)C(=O)NCCc1ccc(-c2ccccc2)cc1]
or a pharmaceutically acceptable salt and/or solvate thereof.
